SEQUENCE LISTING (27 sequences)

SEQ ID NO: 1 - protein, 15 aa, Homo sapiens
  see specification as filed for detailed description of substitutions and preferred embodiments
AGEDPHSFYFPGQFA

SEQ ID NO: 2 - protein, 4 aa, Artificial Sequence
  Description of Artificial Sequence Synthetic peptide
FYFP

SEQ ID NO: 3 - protein, 163 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 4 - protein, 147 aa, Homo sapiens
VGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 5 - protein, 162 aa, Mus sp.
MKCLLISLALWLGTVGTRGTEPELSETQRRSLQVALEEFHKHPPVQLAFQEIGVDRAEEVLFSAGTFVRLEFKLQQTNCPKKDWKKPECTIKPNGRRRKCLACIKMDPKGKILGRIVHCPILKQGPQDPQELQCIKIAQAGEDPHGYFLPGQFAFSRALRTK

SEQ ID NO: 6 - protein, 143 aa, Mus sp.
TEPELSETQRRSLQVALEEFHKHPPVQLAFQEIGVDRAEEVLFSAGTFVRLEFKLQQTNCPKKDWKKPECTIKPNGRRRKCLACIKMDPKGKILGRIVHCPILKQGPQDPQELQCIKIAQAGEDPHGYFLPGQFAFSRALRTK

SEQ ID NO: 7 - protein, 144 aa, Rattus sp.
TELELSETQRRGLQVALEEFHRHPPVQWAFQEIGVDSADDLFFSAGTFVRLEFKLQQTSCLKKDWKKPECTIKPNGRKRKCLACIKLDPKGKVLGRMVHCPILKQGPQQEPQESQCSKIAQAGEDSRIYFFPGQFAFSRALQSK

SEQ ID NO: 8 - protein, 163 aa, Mus sp.
MKCLLISLALWLGTADIHGTELELSETQRRGLQVALEEFHRHPPVQWAFQEIGVDSADDLFFSAGTFVRLEFKLQQTSCLKKDWKKPECTIKPNGRKRKCLACIKLDPKGKVLGRMVHCPILKQGPQQEPQESQCSKIAQAGEDSRIYFFPGQFAFSRALQSK

SEQ ID NO: 9 - protein, 15 aa, Mus sp.
AGEDPHGYFLPGQFA

SEQ ID NO: 10 - protein, 4 aa, Artificial Sequence
  Description of Artificial Sequence Synthetic peptide
FYAP

SEQ ID NO: 11 - protein, 4 aa, Artificial Sequence
  Description of Artificial Sequence Synthetic peptide
YFAP

SEQ ID NO: 12 - protein, 4 aa, Artificial Sequence
  Description of Artificial Sequence Synthetic peptide
YFLP

SEQ ID NO: 13 - protein, 15 aa, Artificial Sequence
  Description of Artificial Sequence Synthetic peptide
AGEDPHGYYFPGQFA

SEQ ID NO: 14 - protein, 15 aa, Artificial Sequence
  Description of Artificial Sequence Synthetic peptide
AGEDPHSXXXPGQFA

SEQ ID NO: 15 - protein, 15 aa, Artificial Sequence
  Description of Artificial Sequence Synthetic peptide
AGEDPHSXXXPGQFA

SEQ ID NO: 16 - protein, 6 aa, Artificial Sequence
  Description of Artificial Sequence Synthetic peptide
PTFYFP

SEQ ID NO: 17 - protein, 14 aa, Artificial Sequence
  Description of Artificial Sequence Synthetic peptide
AGEDPTFYFPGQFA

SEQ ID NO: 18 - protein, 7 aa, Homo sapiens
PHSFYFP

SEQ ID NO: 19 - protein, 15 aa, Artificial Sequence
  Description of Artificial Sequence Synthetic peptide
AFQGPFYFSHPDEGA

SEQ ID NO: 20 - protein, 15 aa, Artificial Sequence
  Description of Artificial Sequence Synthetic peptide
AGEDPHXFYFPGQFA

SEQ ID NO: 21 - protein, 15 aa, Artificial Sequence
  Description of Artificial Sequence Synthetic peptide
AGEDPHSXYFPGQFA

SEQ ID NO: 22 - protein, 15 aa, Artificial Sequence
  Description of Artificial Sequence Synthetic peptide
AGEDPHSXYXPGQFA

SEQ ID NO: 23 - protein, 15 aa, Artificial Sequence
  Description of Artificial Sequence Synthetic peptide
AGEDPHSXXXPGQFA

SEQ ID NO: 24 - protein, 16 aa, Homo sapiens
AGEDPHSFYFPGQFAF

SEQ ID NO: 25 - protein, 17 aa, Homo sapiens
AGEDPHSFYFPGQFAFS

SEQ ID NO: 26 - protein, 11 aa, Homo sapiens
IPTFYFPRGRP

SEQ ID NO: 27 - protein, 15 aa, Artificial Sequence
  Description of Artificial Sequence Synthetic peptide
AGEDPHGYFAPGQFA

(19) United States
(12) Patent Application Publication
(10) Pub. No.: US 2014/0349943 A1
(43) Pub. Date: Nov. 27, 2014

(54) TOPICAL FORMULATIONS OF CHEMERIN C15 PEPTIDES FOR THE TREATMENT OF DERMATOLOGICAL CONDITIONS

(71) Applicant: Thomas Gadek, Oakland, CA (US)

(72) Inventor: Thomas Gadek, Oakland, CA (US)

(21) Appl. No.: 14/352,296

(22) PCT Filed: Oct. 12, 2012
(86) PCT No.: PCT/US12/60093

§ 371 Date: Aug. 14, 2014

Related U.S. Application Data
(60) Provisional application No. 61/546,833, filed on Oct. 13, 2011.

(51) Int. Cl. A61K 38/10; A61K 45/06

(52) U.S. Cl. CPC A61K 38/10; A61K 45/06
USPC 514/18.7

(57) ABSTRACT

Described herein, are topical formulations for treating a dermatological disease, disorder, or condition. Topical formulation disclosed herein include a therapeutically-effective amount of a human chemerin C15 peptide formulated for dermal administration.

### CROSS-REFERENCE

This application is the National Stage Entry of International Application No. PCT/US2012/060093, filed Oct. 12, 2012, which claims priority to U.S. Provisional Patent Application No. 61/546,833, titled “Highly potent antagonists of immune cells in the treatment of skin disorders” and filed 13 Oct. 13, 2011, both of which is are incorporated herein by reference in their entireties.

### SEQUENCE LISTING

The instant application contains a Sequence Listing which has been submitted electronically in ASCII format and is hereby incorporated by reference in its entirety. Said ASCII copy, created on Aug. 14, 2014, is named 41033701831SEQ.txt and is 14,440 bytes in size.

### SUMMARY OF THE INVENTION

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Described herein, in certain embodiments, are topical formulations for treating a dermatological disorder (i.e., an abnormal state of the epidermis, dermis, and/or subcutaneous tissues). Described herein, in certain embodiments, are topical formulations for treating an immune disorder (e.g. an autoimmune disorder (e.g., eczema, psoriasis)); a proliferative disorder (e.g., melanoma); contact with an allergen (e.g., uruishol), and/or an irritant (e.g., alcohol, xylene, turpentine, esters, acetone, ketones); an overproduction of sebum lipids (e.g., acne); a fibroblast disorder (e.g., scarring); or combinations thereof. Described herein, in certain embodiments, are topical formulations for treating psoriasis, atopic dermatitis, contact dermatitis, eczematous dermatitis, alopecia greata, scleredoma, a bullous disorder, acne, urticaria, rosacea, scar formation, and/or melanoma. In some embodiments, a topical formulation disclosed herein comprises a therapeutically-effective amount of a chemerin C15 peptide. In some embodiments, a topical formulation disclosed herein is administered before or after contact with an allergen and/or irritant. In some embodiments, a topical formulation disclosed herein is administered before or after a physical trauma (e.g., surgery).

Described herein, in certain embodiments, is a topical formulation comprising: (a) a chemerin C15 peptide in an amount effective for the treatment of an inflammatory dermatological disorder; and (b) a pharmaceutically acceptable excipient for topical administration, wherein the formulation minimizes systemic exposure. In some embodiments of the topical formulations provided herein, the amount of chemerin C15 peptide is effective for inhibiting secretion of one or more inflammatory cytokines by an antigen presenting cell. In some embodiments of the topical formulations provided herein, the amount of chemerin C15 peptide is effective for inhibiting NFκB nuclear translocation or NFκB-mediated gene transcription of an inflammatory cytokine in an antigen presenting cell. In some embodiments of the topical formulations provided herein, the inflammatory cytokine is IL-23, TNFα, IL-1β, IL-6 or RANTES. In some embodiments of the topical formulations provided herein, the inflammatory cytokine is IL-23. In some embodiments of the topical formulations provided herein, the inflammatory cytokine is TNFα. In some embodiments of the topical formulations provided herein, the inflammatory cytokine is IL-1β. In some embodiments of the topical formulations provided herein, the inflammatory cytokine is RANTES. In some embodiments of the topical formulations provided herein, the antigen presenting cell is an activated macrophage cell, myeloid dendritic cell, or plasmacytoid dendritic cell. In some embodiments of the topical formulations provided herein, the dermatological disorder is an immune disorder, a proliferative disorder, contact with an allergen and/or an irritant, an overproduction of sebum lipids; a fibroblast disorder, or a combination thereof. In some embodiments of the topical formulations provided herein, the dermatological disorder is psoriasis, atopic dermatitis, contact dermatitis, eczematous dermatitis, alopecia greata, scleredoma, a bullous disorder, acne, urticaria, rosacea, scar formation, or melanoma. In some embodiments of the topical formulations provided herein, wherein the dermatological disorder is psoriasis. In some embodiments of the topical formulations provided herein, wherein the dermatological disorder is dermatitis. In some embodiments of the topical formulations provided herein, the dermatological disorder is atopic dermatitis. In some embodiments of the topical formulations provided herein, the dermatological disorder is contact dermatitis. In some embodiments of the topical formulations provided herein, the chemerin C15 peptide is a human chemerin C15 peptide. In some embodiments of the topical formulations provided herein, human chemerin C15 peptide comprises the sequence of amino acids AGEDPHSFYFPGQFA (SEQ ID NO: 1). In some embodiments of the topical formulations provided herein, the human chemerin C15 peptide consists essentially of the sequence of amino acids AGEDPHSFYFPGQFA (SEQ ID NO: 1). In some embodiments of the topical formulations provided herein, the topical formulation is formulated as an ointment. In some embodiments of the topical formulations provided herein, the ointment comprises about 1-10 mg of the chemerin C15 peptide per gram of ointment. In some embodiments of the topical formulations provided herein, the ointment comprises petrolatum. In some embodiments of the topical formulations provided herein, the ointment comprises caprylic capric triglyceride. In some embodiments of the topical formulations provided herein, the ointment comprises beeswax. In some embodiments of the topical formulations provided herein, the ointment comprises petrolatum, caprylic triglyceride and beeswax. In some embodiments of the topical formulations provided herein, the ointment comprises about 50% petrolatum, about 45% caprylic triglyceride and about 5% beeswax. In some embodiments of the topical formulations provided herein, the ointment comprises butylated hydroxytoluene, PEG 400, Span 80, white wax, and white petrolatum. In some embodiments of the topical formulations provided herein, the ointment comprises about 0.02% w/w butylated hydroxytoluene, about 15% w/w PEG 400, about 2% w/w Span 80, about 10% w/w white wax, and about 71.98% w/w white petrolatum. In some embodiments of the topical formulations provided herein, the ointment comprises butylated dimethyl isosorbide, butylated hydroxytoluene, Span 80, white wax, and white petrolatum. In some embodiments of the topical formulations provided herein, the ointment comprises about 10% w/w dimethyl isosorbide, about 0.02% w/w butylated hydroxytoluene, about 2% w/w Span 80, about 10% w/w white wax, and about 76.98% w/w white petrolatum. In some embodiments of the topical formulations provided herein, the topical formulation is formulated as a solution. In some embodiments of the topical formulations provided herein, the topical formulation is formulated as a solution that is applied as a spray. In some embodiments of the topical formulations provided herein, the solution comprises about 1-10 mg of the chemerin C15 peptide per ml of solution. In some embodiments of the topical formulations provided herein, the solution comprises isopropyl myristate, alcohol, undecylenic acid and sodium lauryl sulfate. In some embodiments of the topical formulations provided herein, the solution comprises about 45% isopropyl myristate, about 45% alcohol, about 5% undecylenic acid and about 5% sodium lauryl sulfate. In some embodiments of the topical formulations provided herein, the solution comprises DMSO. In some embodiments of the topical formulations provided herein, the solution comprises about 50% DMSO, and about 50% water. In some embodiments of the topical formulations provided herein, the solution comprises dimethyl isosorbide, Transcutol, hexylene glycol, and propylene glycol. In some embodiments of the topical formulations provided herein, the solution comprises about 15% w/w dimethyl isosorbide, about 25% w/w Transcutol, about 12% w/w hexylene glycol, and about 5% w/w propylene glycol. In some embodiments of the topical formulations provided herein, the topical formulation is formulated as a cream. In some embodiments of the topical formulations provided herein, the cream comprises about 1-10 mg of the chemerin C15 peptide per ml of cream. In some embodiments of the topical formulations provided herein, the topical formulation is formulated as a lotion. In some embodiments of the topical formulations provided herein, the lotion comprises about 1-10 mg of the chemerin C15 peptide per ml of lotion. In some embodiments of the topical formulations provided herein, the lotion comprises Dimethyl isosorbide, Transcutol, Hexylene glycol, Propylene glycol, Methylparaben, Propylparaben, EDTA, Carbopol Ultrez 10, Penmulen TR-1, and Butylated hydroxytoluene. In some embodiments of the topical formulations provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Isopropyl myristate, Oleyl alcohol, Butylated hydroxytoluene, and White petrolatum. In some embodiments of the topical formulations provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.5% w/w Carbopol Ultrez 10, about 0.2% w/w Penmulen TR-1, about 3% w/w Isopropyl myristate, about 5% w/w Oleyl alcohol, about 0.2% w/w Butylated hydroxytoluene, and about 5% w/w White petrolatum. In some embodiments of the topical formulations provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Cetyl alcohol, Light mineral oil, Oleic acid, Butylated hydroxytoluene. In some embodiments of the topical formulations provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.3% w/w Carbopol Ultrez 10, about 0.2% w/w Penmulen TR-1, about 2% w/w Cetyl alcohol, about 5.5% w/w Light mineral oil, about 5% w/w Oleic acid, and about 0.2% w/w Butylated hydroxytoluene. In some embodiments of the topical formulations provided herein, the topical formulation comprises a skin penetration agent. In some embodiments of the topical formulations provided herein, the skin penetration agent is DMSO. In some embodiments of the topical formulations provided herein, the topical formulation comprises a gelling agent. In some embodiments of the topical formulations provided herein, the topical formulation comprises an emollient. In some embodiments of the topical formulations provided herein, the topical formulation comprises an anti-oxidant. In some embodiments of the topical formulations provided herein, the topical formulation comprises a skin protecting agent. In some embodiments of the topical formulations provided herein, the topical formulation comprises an irritation-mitigating agent. In some embodiments of the topical formulations provided herein, the topical formulation comprises a dry-feel modifier. In some embodiments of the topical formulations provided herein, the topical formulation comprises a surfactant. In some embodiments of the topical formulations provided herein, the topical formulation comprises a preservative. In some embodiments of the topical formulations provided herein, the topical formulation comprises a chelating agent. In some embodiments of the topical formulations provided herein, wherein the topical formulation comprises a lubricant. In some embodiments of the topical formulations provided herein, the topical formulation comprises a thickening agent. In some embodiments of the topical formulations provided herein, the topical formulation comprises at least one additional therapeutic agent. In some embodiments of the topical formulations provided herein, the additional therapeutic agent is an antioxidant, anti-inflammatory agent, antiangiogenic agent, anti-apoptotic agent, vascular endothelial growth factor inhibitor, antimicrobial or antiviral agent. In some embodiments of the topical formulations provided herein, the additional therapeutic agent is a corticosteroid.

Described herein, in certain embodiments, is a topical formulation of a chemerin C15 peptide formulated as an aerosol, liquid, ointment, cream, lotion, solution, spray, suspension, emulsion, paste, gel, powder, salve, plaster, paint, foam, stick, slow release nanoparticle, slow release microparticle, bioadhesive, patch, bandage or wound dressing. In some embodiments of the topical formulations provided herein, the chemerin C15 peptide is a human chemerin C15 peptide. In some embodiments of the topical formulations provided herein, human chemerin C15 peptide comprises the sequence of amino acids AGEDPHSFYFPGQFA (SEQ ID NO: 1). In some embodiments of the topical formulations provided herein, the human chemerin C15 peptide consists essentially of the sequence of amino acids AGEDPHSFYFPGQFA (SEQ ID NO: 1). In some embodiments of the topical formulations provided herein, the topical formulation is formulated as an ointment. In some embodiments of the topical formulations provided herein, the ointment comprises about 1-10 mg of the chemerin C15 peptide per gram of ointment. In some embodiments of the topical formulations provided herein, the ointment comprises petrolatum. In some embodiments of the topical formulations provided herein, the ointment comprises caprylic capric triglyceride. In some embodiments of the topical formulations provided herein, the ointment comprises beeswax. In some embodiments of the topical formulations provided herein, the ointment comprises petrolatum, caprylic triglyceride and beeswax. In some embodiments of the topical formulations provided herein, the ointment comprises about 50% petrolatum, about 45% caprylic triglyceride and about 5% beeswax. In some embodiments of the topical formulations provided herein, the ointment comprises butylated hydroxytoluene, PEG 400, Span 80, white wax, and white petrolatum. In some embodiments of the topical formulations provided herein, the ointment comprises about 0.02% w/w butylated hydroxytoluene, about 15% w/w PEG 400, about 2% w/w Span 80, about 10% w/w white wax, and about 71.98% w/w white petrolatum. In some embodiments of the topical formulations provided herein, the ointment comprises butylated dimethyl isosorbide, butylated hydroxytoluene, Span 80, white wax, and white petrolatum. In some embodiments of the topical formulations provided herein, the ointment comprises about 10% w/w dimethyl isosorbide, about 0.02% w/w butylated hydroxytoluene, about 2% w/w Span 80, about 10% w/w white wax, and about 76.98% w/w white petrolatum. In some embodiments of the topical formulations provided herein, the topical formulation is formulated as a solution. In some embodiments of the topical formulations provided herein, the topical formulation is formulated as a solution that is applied as a spray. In some embodiments of the topical formulations provided herein, the solution comprises about 1-10 mg of the chemerin C15 peptide per ml of solution. In some embodiments of the topical formulations provided herein, the solution comprises isopropyl myristate, alcohol, undecylenic acid and sodium lauryl sulfate. In some embodiments of the topical formulations provided herein, the solution comprises about 45% isopropyl myristate, about 45% alcohol, about 5% undecylenic acid and about 5% sodium lauryl sulfate. In some embodiments of the topical formulations provided herein, the solution comprises DMSO. In some embodiments of the topical formulations provided herein, the solution comprises about 50% DMSO, and about 50% water. In some embodiments of the topical formulations provided herein, the solution comprises dimethyl isosorbide, Transcutol, hexylene glycol, and propylene glycol. In some embodiments of the topical formulations provided herein, the solution comprises about 15% w/w dimethyl isosorbide, about 25% w/w Transcutol, about 12% w/w hexylene glycol, and about 5% w/w propylene glycol. In some embodiments of the topical formulations provided herein, the topical formulation is formulated as a cream. In some embodiments of the topical formulations provided herein, the cream comprises about 1-10 mg of the chemerin C15 peptide per ml of cream. In some embodiments of the topical formulations provided herein, the topical formulation is formulated as a lotion. In some embodiments of the topical formulations provided herein, the lotion comprises about 1-10 mg of the chemerin C15 peptide per ml of lotion. In some embodiments of the topical formulations provided herein, the lotion comprises Dimethyl isosorbide, Transcutol, Hexylene glycol, Propylene glycol, Methylparaben, Propylparaben, EDTA, Carbopol Ultrez 10, Penmulen TR-1, and Butylated hydroxytoluene. In some embodiments of the topical formulations provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Isopropyl myristate, Oleyl alcohol, Butylated hydroxytoluene, and White petrolatum. In some embodiments of the topical formulations provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.5% w/w Carbopol Ultrez 10, about 0.2% w/w Penmulen TR-1, about 3% w/w Isopropyl myristate, about 5% w/w Oleyl alcohol, about 0.2% w/w Butylated hydroxytoluene, and about 5% w/w White petrolatum. In some embodiments of the topical formulations provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Cetyl alcohol, Light mineral oil, Oleic acid, Butylated hydroxytoluene. In some embodiments of the topical formulations provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.3% w/w Carbopol Ultrez 10, about 0.2% w/w Penmulen TR-1, about 2% w/w Cetyl alcohol, about 5.5% w/w Light mineral oil, about 5% w/w Oleic acid, and about 0.2% w/w Butylated hydroxytoluene. In some embodiments of the topical formulations provided herein, the topical formulation comprises a skin penetration agent. In some embodiments of the topical formulations provided herein, the skin penetration agent is DMSO. In some embodiments of the topical formulations provided herein, the topical formulation comprises a gelling agent. In some embodiments of the topical formulations provided herein, the topical formulation comprises an emollient. In some embodiments of the topical formulations provided herein, the topical formulation comprises an anti-oxidant. In some embodiments of the topical formulations provided herein, the topical formulation comprises a skin protecting agent. In some embodiments of the topical formulations provided herein, the topical formulation comprises an irritation-mitigating agent. In some embodiments of the topical formulations provided herein, the topical formulation comprises a dry-feel modifier. In some embodiments of the topical formulations provided herein, the topical formulation comprises a surfactant. In some embodiments of the topical formulations provided herein, the topical formulation comprises a preservative. In some embodiments of the topical formulations provided herein, the topical formulation comprises a chelating agent. In some embodiments of the topical formulations provided herein, wherein the topical formulation comprises a lubricant. In some embodiments of the topical formulations provided herein, the topical formulation comprises a thickening agent. In some embodiments of the topical formulations provided herein, the topical formulation comprises at least one additional therapeutic agent. In some embodiments of the topical formulations provided herein, the additional therapeutic agent is an antioxidant, anti-inflammatory agent, antiangiogenic agent, anti-apoptotic agent, vascular endothelial growth factor inhibitor, antimicrobial or antiviral agent. In some embodiments of the topical formulations provided herein, the additional therapeutic agent is a corticosteroid.

Described herein, in certain embodiments, is a method of treating of an inflammatory dermatological disorder in an individual in need thereof, comprising administering to the individual a therapeutically-effective amount of a topical formulation comprising a human chemerin C15 peptide, wherein the formulation is formulated to minimize systemic exposure to the individual. In some embodiments of the methods provided herein, administration inhibits the secretion one or more inflammatory cytokines by an antigen presenting cell. In some embodiments of the methods provided herein, administration inhibits NFκB nuclear translocation or NFκB-mediated gene transcription of an inflammatory cytokine in an antigen presenting cell. In some embodiments of the methods provided herein, the inflammatory cytokine is IL-23, TNFα, IL-1β, IL-6 or RANTES. In some embodiments of the methods provided herein, the inflammatory cytokine is IL-23. In some embodiments of the methods provided herein, the inflammatory cytokine is TNFα. In some embodiments of the methods provided herein, the inflammatory cytokine is IL-1β. In some embodiments of the methods provided herein, the inflammatory cytokine is RANTES. In some embodiments of the methods provided herein, the antigen presenting cell is an activated macrophage cell, myeloid dendritic cell, a plasmacytoid dendritic cell. In some embodiments of the methods provided herein, the chemerin C15 peptide comprises the sequence of amino acids AGEDPHSFYFPGQFA (SEQ ID NO: 1). In some embodiments of the methods provided herein, the chemerin C15 peptide consists essentially of the sequence of amino acids AGEDPHSFYFPGQFA (SEQ ID NO: 1). In some embodiments of the methods provided herein, the dermatological disorder is an immune disorder, a proliferative disorder, contact with an allergen and/or an irritant, an overproduction of sebum lipids; a fibroblast disorder, or a combination thereof. In some embodiments of the methods provided herein, the dermatological disorder is psoriasis, atopic dermatitis, contact dermatitis, eczematous dermatitis, alopecia greata, scleredoma, a bullous disorder, acne, urticaria, rosacea, scar formation, or melanoma. In some embodiments of the methods provided herein, the dermatological disorder is psoriasis. In some embodiments of the methods provided herein, the dermatological disorder is dermatitis. In some embodiments of the methods provided herein, the dermatological disorder is atopic dermatitis. In some embodiments of the methods provided herein, the dermatological disorder is contact dermatitis. In some embodiments of the methods provided herein, the topical formulation is in the form of an aerosol, liquid, ointment, cream, lotion, solution, suspension, emulsion, paste, gel, powder, salve, plaster, paint, foam, stick, slow release nanoparticle, slow release microparticle, bioadhesive, patch, bandage or wound dressing. In some embodiments of the methods provided herein, the formulation is formulated as an ointment. In some embodiments of the methods provided herein, the ointment comprises about 1-10 mg of the chemerin C15 peptide per gram of ointment. In some embodiments of the methods provided herein, the ointment comprises petrolatum. In some embodiments of the methods provided herein, the ointment comprises caprylic capric triglyceride. In some embodiments of the methods provided herein, the ointment comprises beeswax. In some embodiments of the methods provided herein, the ointment comprises petrolatum, caprylic triglyceride and beeswax. In some embodiments of the methods provided herein, the ointment comprises about 50% petrolatum, about 45% caprylic triglyceride and about 5% beeswax. In some embodiments of the methods provided herein, the ointment comprises butylated hydroxytoluene, PEG 400, Span 80, white wax, and white petrolatum. In some embodiments of the methods provided herein, the ointment comprises about 0.02% w/w butylated hydroxytoluene, about 15% w/w PEG 400, about 2% w/w Span 80, about 10% w/w white wax, and about 71.98% w/w white petrolatum. In some embodiments of the methods provided herein, the ointment comprises butylated dimethyl isosorbide, butylated hydroxytoluene, Span 80, white wax, and white petrolatum. In some embodiments of the methods provided herein, the ointment comprises about 10% w/w dimethyl isosorbide, about 0.02% w/w butylated hydroxytoluene, about 2% w/w Span 80, about 10% w/w white wax, and about 76.98% w/w white petrolatum. In some embodiments of the methods provided herein, the formulation is formulated as a solution. In some embodiments of the methods provided herein, the formulation is formulated as a solution that is applied as a spray. In some embodiments of the methods provided herein, the solution comprises about 1-10 mg of the chemerin C15 peptide per ml of solution. In some embodiments of the methods provided herein, the solution comprises isopropyl myristate, alcohol, undecylenic acid and sodium lauryl sulfate. In some embodiments of the methods provided herein, the solution comprises about 45% isopropyl myristate, about 45% alcohol, about 5% undecylenic acid and about 5% sodium lauryl sulfate. In some embodiments of the methods provided herein, the solution comprises DMSO. In some embodiments of the methods provided herein, the solution comprises about 50% DMSO, and about 50% water. In some embodiments of the methods provided herein, the solution comprises dimethyl isosorbide, Transcutol, hexylene glycol, and propylene glycol. In some embodiments of the methods provided herein, solution comprises about 15% w/w dimethyl isosorbide, about 25% w/w Transcutol, about 12% w/w hexylene glycol, and about 5% w/w propylene glycol. In some embodiments of the methods provided herein, the formulation is formulated as a cream. In some embodiments of the methods provided herein, the cream comprises about 1-10 mg of the chemerin C15 peptide per ml of cream. In some embodiments of the methods provided herein, the formulation is formulated as a lotion. In some embodiments of the methods provided herein, the lotion comprises about 1-10 mg of the chemerin C15 peptide per ml of lotion. In some embodiments of the methods provided herein, the lotion comprises Dimethyl isosorbide, Transcutol, Hexylene glycol, Propylene glycol, Methylparaben, Propylparaben, EDTA, Carbopol Ultrez 10, Penmulen TR-1, and Butylated hydroxytoluene. In some embodiments of the methods provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Isopropyl myristate, Oleyl alcohol, Butylated hydroxytoluene, and White petrolatum. In some embodiments of the methods provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.5% w/w Carbopol Ultrez 10, about 0.2% w/w Penmulen TR-1, about 3% w/w Isopropyl myristate, about 5% w/w Oleyl alcohol, about 0.2% w/w Butylated hydroxytoluene, and about 5% w/w White petrolatum. In some embodiments of the methods provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Cetyl alcohol, Light mineral oil, Oleic acid, Butylated hydroxytoluene. In some embodiments of the methods provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.3% w/w Carbopol Ultrez 10, about 0.2% w/w Penmulen TR-1, about 2% w/w Cetyl alcohol, about 5.5% w/w Light mineral oil, about 5% w/w Oleic acid, and about 0.2% w/w Butylated hydroxytoluene. In some embodiments of the methods provided herein, the topical formulation comprises a skin penetration agent. In some embodiments of the methods provided herein, the skin penetration agent is DMSO. In some embodiments of the methods provided herein, the topical formulation comprises a gelling agent. In some embodiments of the methods provided herein, the topical formulation comprises an emollient. In some embodiments of the methods provided herein, the topical formulation comprises an anti-oxidant. In some embodiments of the methods provided herein, the topical formulation comprises a skin protecting agent. In some embodiments of the methods provided herein, the topical formulation comprises an irritation-mitigating agent. In some embodiments of the methods provided herein, the topical formulation comprises a dry-feel modifier. In some embodiments of the methods provided herein, the topical formulation comprises a surfactant. In some embodiments of the methods provided herein, the topical formulation comprises a preservative. In some embodiments of the methods provided herein, the topical formulation comprises a chelating agent. In some embodiments of the methods provided herein, the topical formulation comprises a lubricant. In some embodiments of the methods provided herein, the topical formulation comprises a thickening agent. In some embodiments of the methods provided herein, the topical formulation comprises at least one additional therapeutic agent. In some embodiments of the methods provided herein, the additional therapeutic agent is an antioxidant, anti-inflammatory agent, antiangiogenic agent, anti-apoptotic agent, vascular endothelial growth factor inhibitor, antimicrobial or antiviral agent. In some embodiments of the methods provided herein, the additional therapeutic agent is a corticosteroid. In some embodiments of the methods provided herein, the topical formulation is topically applied to the skin, eye, mouth, nose, vaginal mucosa or anal mucosa. In some embodiments of the methods provided herein, administration of the topical formulation results in a local tissue concentration of the chemerin C15 peptide of greater than about 0.1 pM-100 nM, greater than about 1 pM-10 nM, greater than about 1 pM-1 nM, greater than about 1-100 pM, or greater than about 1-10 pM at about 1-12 hours following administration to the individual. In some embodiments of the methods provided herein, administration of the topical formulation results in a systemic concentration of the chemerin C15 peptide of less than about 100 pM, less than about 10 pM, less than about 1 pM, less than about 0.1 pM, or less than about 0.01 pM.

Described herein, in certain embodiments, is a use of a human chemerin C15 peptide for the manufacture of a topical formulation comprising a therapeutically-effective amount of the peptide for treating an inflammatory dermatological disorder, wherein the formulation is formulated to minimize systemic exposure. In some embodiments of the uses provided herein, the amount of the human chemerin C15 peptide is effective for inhibiting the secretion one or more inflammatory cytokines by an antigen presenting cell. In some embodiments of the uses provided herein, the amount of the human chemerin C15 peptide is effective for inhibiting the NFκB nuclear translocation or NFκB-mediated gene transcription of an inflammatory cytokine in an antigen presenting cell. In some embodiments of the uses provided herein, the inflammatory cytokine is IL-23, TNFα, IL-1β, IL-6 or RANTES. In some embodiments of the uses provided herein, the inflammatory cytokine is IL-23. In some embodiments of the uses provided herein, the inflammatory cytokine is TNFα. In some embodiments of the uses provided herein, the inflammatory cytokine is IL-1β. In some embodiments of the uses provided herein, the inflammatory cytokine is RANTES. In some embodiments of the uses provided herein, the antigen presenting cell is an activated macrophage cell, myeloid dendritic cell, a plasmacytoid dendritic cell. In some embodiments of the uses provided herein, the chemerin C15 peptide comprises the sequence of amino acids AGEDPHSFYFPGQFA (SEQ ID NO: 1). In some embodiments of the uses provided herein, the wherein the chemerin C15 peptide consists essentially of the sequence of amino acids AGEDPHSFYFPGQFA (SEQ ID NO: 1). In some embodiments of the uses provided herein, the dermatological disorder is an immune disorder, a proliferative disorder, contact with an allergen and/or an irritant, an overproduction of sebum lipids; a fibroblast disorder, or a combination thereof. In some embodiments of the uses provided herein, the dermatological disorder is psoriasis, atopic dermatitis, contact dermatitis, eczematous dermatitis, alopecia greata, scleredoma, a bullous disorder, acne, urticaria, rosacea, scar formation, or melanoma. In some embodiments of the uses provided herein, the dermatological disorder is psoriasis. In some embodiments of the uses provided herein, the dermatological disorder is dermatitis. In some embodiments of the uses provided herein, the dermatological disorder is atopic dermatitis. In some embodiments of the uses provided herein, the dermatological disorder is contact dermatitis. In some embodiments of the uses provided herein, the topical formulation is in the form of an aerosol, liquid, ointment, cream, lotion, solution, suspension, emulsion, paste, gel, powder, salve, plaster, paint, foam, stick, slow release nanoparticle, slow release microparticle, bioadhesive, patch, bandage or wound dressing. In some embodiments of the uses provided herein, the topical formulation is formulated as an ointment. In some embodiments of the uses provided herein, the ointment comprises about 1-10 mg of the chemerin C15 peptide per gram of ointment. In some embodiments of the uses provided herein, the ointment comprises petrolatum. In some embodiments of the uses provided herein, the ointment comprises caprylic capric triglyceride. In some embodiments of the uses provided herein, the ointment comprises beeswax. In some embodiments of the uses provided herein, the ointment comprises petrolatum, caprylic triglyceride and beeswax. In some embodiments of the uses provided herein, the ointment comprises about 50% petrolatum, about 45% caprylic triglyceride and about 5% beeswax. In some embodiments of the uses provided herein, the ointment comprises butylated hydroxytoluene, PEG 400, Span 80, white wax, and white petrolatum. In some embodiments of the uses provided herein, the ointment comprises about 0.02% w/w butylated hydroxytoluene, about 15% w/w PEG 400, about 2% w/w Span 80, about 10% w/w white wax, and about 71.98% w/w white petrolatum. In some embodiments of the uses provided herein, the ointment comprises butylated dimethyl isosorbide, butylated hydroxytoluene, Span 80, white wax, and white petrolatum. In some embodiments of the uses provided herein, the ointment comprises about 10% w/w dimethyl isosorbide, about 0.02% w/w butylated hydroxytoluene, about 2% w/w Span 80, about 10% w/w white wax, and about 76.98% w/w white petrolatum. In some embodiments of the uses provided herein, the topical formulation is formulated as a solution. In some embodiments of the uses provided herein, the topical formulation is formulated as a solution that is applied as a spray. In some embodiments of the uses provided herein, the solution comprises about 1-10 mg of the chemerin C15 peptide per ml of solution. In some embodiments of the uses provided herein, the solution comprises isopropyl myristate, alcohol, undecylenic acid and sodium lauryl sulfate. In some embodiments of the uses provided herein, the solution comprises about 45% isopropyl myristate, about 45% alcohol, about 5% undecylenic acid and about 5% sodium lauryl sulfate. In some embodiments of the uses provided herein, the solution comprises DMSO. In some embodiments of the uses provided herein, the solution comprises about 50% DMSO, and about 50% water. In some embodiments of the uses provided herein, the solution comprises dimethyl isosorbide, Transcutol, hexylene glycol, and propylene glycol. In some embodiments of the uses provided herein, the solution comprises about 15% w/w dimethyl isosorbide, about 25% w/w Transcutol, about 12% w/w hexylene glycol, and about 5% w/w propylene glycol. In some embodiments of the uses provided herein, the topical formulation is formulated as a cream. In some embodiments of the uses provided herein, the cream comprises about 1-10 mg of the chemerin C15 peptide per ml of cream. In some embodiments of the uses provided herein, the topical formulation is formulated as a lotion. In some embodiments of the uses provided herein, the lotion comprises about 1-10 mg of the chemerin C15 peptide per ml of lotion. In some embodiments of the uses provided herein, the lotion comprises Dimethyl isosorbide, Transcutol, Hexylene glycol, Propylene glycol, Methylparaben, Propylparaben, EDTA, Carbopol Ultrez 10, Penmulen TR-1, and Butylated hydroxytoluene. In some embodiments of the uses provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Isopropyl myristate, Oleyl alcohol, Butylated hydroxytoluene, and White petrolatum. In some embodiments of the uses provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.5% w/w Carbopol Ultrez 10, about 0.2% w/w Penmulen TR-1, about 3% w/w Isopropyl myristate, about 5% w/w Oleyl alcohol, about 0.2% w/w Butylated hydroxytoluene, and about 5% w/w White petrolatum. In some embodiments of the uses provided herein, the lotion comprises Dimethyl isosorbide, Transcutol, Hexylene glycol, Propylene glycol, Methylparaben, Propylparaben, EDTA, Carbopol Ultrez 10, Penmulen TR-1, Cetyl alcohol, Light mineral oil, Oleic acid, Butylated hydroxytoluene. In some embodiments of the uses provided herein, the lotion comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.3% w/w Carbopol Ultrez 10, about 0.2% w/w Penmulen TR-1, about 2% w/w Cetyl alcohol, about 5.5% w/w Light mineral oil, about 5% w/w Oleic acid, and about 0.2% w/w Butylated hydroxytoluene. In some embodiments of the uses provided herein, the topical formulation comprises a skin penetration agent. In some embodiments of the uses provided herein, the skin penetration agent is DMSO. In some embodiments of the uses provided herein, the topical formulation comprises a gelling agent. In some embodiments of the uses provided herein, the topical formulation comprises an emollient. In some embodiments of the uses provided herein, the topical formulation comprises an anti-oxidant. In some embodiments of the uses provided herein, the topical formulation comprises a skin protecting agent. In some embodiments of the uses provided herein, the topical formulation comprises an irritation-mitigating agent. In some embodiments of the uses provided herein, the topical formulation comprises a dry-feel modifier. In some embodiments of the uses provided herein, the topical formulation comprises a surfactant. In some embodiments of the uses provided herein, the topical formulation comprises a preservative. In some embodiments of the uses provided herein, the topical formulation comprises a chelating agent. In some embodiments of the uses provided herein, the topical formulation comprises a lubricant. In some embodiments of the uses provided herein, the topical formulation comprises a thickening agent. In some embodiments of the uses provided herein, the topical formulation comprises at least one additional therapeutic agent. In some embodiments of the uses provided herein, the additional therapeutic agent is an antioxidant, anti-inflammatory agent, antiangiogenic agent, anti-apoptotic agent, vascular endothelial growth factor inhibitor, antimicrobial or antiviral agent. In some embodiments of the uses provided herein, the additional therapeutic agent is a corticosteroid. In some embodiments of the uses provided herein, the topical formulation is formulated for application to the skin, eye, mouth, nose, vaginal mucosa or anal mucosa.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies the effect of human chemerin C15 and C17 peptides on cytokine production in IFNγ/LPS stimulated human macrophages. A) IL-1β at 15 hours; B) RANTES at 15 hours; C) RANTES (Difference from 6 to 15 hours); D) IL-12p40 at 15 hours; and E) IL-10 at 15 hours.

FIG. 2 exemplifies agonist and antagonist dose response curves for ChemR23 and GPR1 receptors in the presence of chemerin, human chemerin C15, 16, or C17 peptides (SEQ ID NOS 1 and 24-25, respectively), or mouse chemerin C15 peptide (SEQ ID NO: 9).

FIG. 3 exemplifies loss of human chemerin C15 peptide anti-inflammatory activity by modification of the FYFP motif (SEQ ID NO: 2). FIG. 3 discloses SEQ ID NOS 1, 9, 27, and 16, respectively, in order of appearance)

### DETAILED DESCRIPTION OF THE INVENTION

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

### Certain Terminology

As used herein, “chemerin C15 peptide” refers to a peptide that comprises the sequence of amino acids AGEDPHSFYFPGQFA (SEQ ID NO: 1) of a human chemerin polypeptide, a species variant of the human chemerin C15 peptide, such as a mouse or rat chemerin C15 peptide, or other variants of the human chemerin C15 peptide as described herein.

As used herein, “peptide” is intended to have its art recognized meaning, i.e., two or more amino acids linked through amide bonds, for example, repeating units of formula —C(═O)CH(side chain)NH—that, in the simplest form, terminate in either an amine or a carboxylic acid. As one of ordinary skill in the art will recognize, numerous modifications of the peptidic backbone are possible without changing the overall nature of the molecule, including modification of the terminal groups such as those described herein.

As used herein, “amino acid” is intended to have its art-recognized meaning, i.e., a carboxylic acid of general formula HOC(═O)CH(side chain)(NH₂). Side chains of amino acids are well known in the art and include naturally occurring and non-naturally occurring moieties. Non-naturally occurring (i.e., unnatural) amino acid side chains are moieties that are used in place of naturally occurring amino acid side chains in, for example, amino acid analogs.

The terms “individual,” “patient,” or “subject” are used interchangeably. As used herein, they mean any mammal. In one aspect, the mammal is a human. None of the terms require that the individual/patient/subject is under the care of a medical professional (e.g., a doctor, nurse, physician's assistant, registered nurse, nurse practitioner, hospice worker, orderly, etc.).

The terms “treat,” “treating” or “treatment,” and other grammatical equivalents as used herein, include alleviating, abating, inhibiting, reducing, ameliorating, delaying the onset of, arresting the progression of, and/or inducing the regression of a disorder and/or the symptoms of a disorder. The terms also include prophylactic treatment of a disorder. The terms further include achieving any therapeutic benefit. Therapeutic benefit means the eradication or amelioration of the underlying disorder being treated, and/or the eradication or amelioration of one or more of the physiological symptoms associated with the underlying disorder such that an improvement is observed and/or perceived in the individual.

The terms “prevent,” “preventing” or “prevention,” and other grammatical equivalents as used herein include inhibiting (arresting or stopping) the development of a disorder, and/or inhibiting (arresting or stopping) the further progression of a disorder. These terms are intended to include prophylaxis. For prophylactic benefit, the compositions are administered to an individual at risk of developing a particular disorder, or to an individual reporting one or more of the physiological symptoms of a disease, or to an individual at risk of reoccurrence of the disease.

The terms “effective amount” or “therapeutically effective amount” as used herein, refer to an amount of an agent (e.g. a chemerin C15 peptide) being administered which achieves a desired result, e.g., to relieve to some extent one or more symptoms of a disease, disorder or condition being treated. In certain instances, the result is a reduction and/or alleviation of at least one sign, symptom, or cause of a disease, or any other desired alteration of a biological system. In certain instances, an “effective amount” for therapeutic uses is the amount of the composition comprising an agent as set forth herein required to provide a clinically significant decrease in at least one symptom of a disease, disorder or condition. An appropriate “effective” amount in any individual case is determined using any suitable technique, such as a dose escalation study. For example, as used herein an appropriate effective amount of a topical agent (e.g. a chemerin C15 peptide) applied locally to a tissue is an amount sufficient to achieve a local therapeutic concentration which has been shown in vitro to inhibit a cellular process associated with inflammation, such as, for example, inhibition of NFκB and/or inhibition of the production and/or secretion of one or more inflammatory cytokines.

The terms “administer,” “administering,” “administration,” and the like, as used herein, refer to the methods that are used to enable delivery of chemerin C15 peptides to the desired site of biological action (e.g., the site of a dermal disorder). These methods include any suitable method for dermatological (i.e., topical) administration.

As used herein, the terms “formulation” and “composition” are used interchangeably. They mean a product comprising a chemerin C15 peptide disclosed herein and a pharmaceutically-acceptable excipient.

As used herein, “topical” administration refers administration to the skin, eye or a mucosal surface, such as an oral, nasal, vaginal or anal surface, of the subject.

“Localized treatment” as used herein refers to treatment of an immune or inflammatory disorder wherein the drug is delivered locally and is not delivered via systemic delivery. In some embodiments, this includes many different local areas or a few different local areas within, for example, treatment of skin, wherein the drug is applied to many different locations or a few different locations on the skin, and wherein drug is delivered to tissues within and adjacent to the skin by absorption through the skin. In some embodiments, drug is delivered to a mucosal surface, such as the mouth, nose, anus or vagina, and absorbed through the epithelial surfaces of the tissue within and adjacent to the mucosa.

“Local tissue concentration” as used herein, refers to the concentration of the chemerin C15 peptide within the tissue area to which the chemerin C15 peptides has been delivered and absorbed.

The term “pharmaceutically acceptable” as used herein, refers to a material that does not abrogate the biological activity or properties of the agents described herein, and is relatively nontoxic (i.e., the toxicity of the material does not significantly outweigh the benefit of the material).

### Overview of Chemerin C-Terminal Peptides and Inflammatory Skin Disorders

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Skin disorders are, in certain instances, marked by increased inflammation in the skin. In certain instances, skin disorders result from the infiltration of inflammatory cells including macrophages, dendritic cells, monocytes, neutrophils and NK cells into skin tissue. Antigen presentation from these cells activate auto-reactive T-cells in skin diseases. Currently approved therapies for skin disorders include antibodies and biological agents targeting cytokines including, for example, TNFα, IL-12, IL-23, IL-1β and/or IL-6. Efficacy of these agents has been linked to a reduction in levels of TNFα, IL-12, IL-23, IL-1β and/or IL-6 in diseased skin tissue. Additional cytokines linked to inflammatory skin disorders and diseases include but are not limited to IL-1, IL-2, IL-3, IL-4, IL-5, IL-6, IL-7, IL-8, IL-9, IL-11, IL-12, IL-13, IL-14, IL-15, IL-16, IL-17, IL-18, IL-19, IL-20, IL-21, IL-22, IL-23, IL-24, Il-25, IL-26, IL-27, IL-28, IL-29, IL-30, as well as TNF family members, IFN family members, RANTES, MCP-1, and MIP-1. These anti-cytokine antibodies and biological agents are typically administered systemically and as such lead to systemic immunosuppression which places the patient at increased risk for unintended side effects including increased infections and death. In one example, the monoclonal antibody, Raptiva, an approved psoriasis treatment, was removed from the market after several cases of PML and death were linked to its use.

Chemerin, also known as retinoic acid receptor responder protein 2 (RARRES2), tazarotene-induced gene 2 protein (TIG2), or RAR-responsive protein TIG2, is a 157 amino acid plasma protein derived from enzymatic cleavage of its 163 amino acid precursor, prochemerin.

Human prochemerin has the amino acid sequence:

(SEQ ID NO: 3)
MRRLLIPLALWLGAVGVGVAELTEAQRRGLQVALEEFHKHP
PVQWAFQETSVESAVDTPFPAGIFVRLEFKLQQTSCRKRDW
KKPECKVRPNGRKRKCLACIKLGSEDKVLGRLVHCPIETQV
LREAEEHQETQCLRVQRAGEDPHSFYFPGQFAFSKALPRS.

Mature human chemerin has the amino acid sequence:

(SEQ ID NO: 4)
VGVAELTEAQRRGLQVALEEFHKHPPVQWAFQETSVESAVD
TPFPAGIFVRLEFKLQQTSCRKRDWKKPECKVRPNGRKRKC
LACIKLGSEDKVLGRLVHCPIETQVLREAEEHQETQCLRVQ
RAGEDPHSFYFPGQFAFSKALPRS.

Mouse prochemerin has the amino acid sequence:

(SEQ ID NO: 5)
MKCLLISLALWLGTVGTRGTEPELSETQRRSLQVALEEFHKH
PPVQLAFQEIGVDRAEEVLFSAGTFVRLEFKLQQTNCPKKDW
KKPECTIKPNGRRRKCLACIKMDPKGKILGRIVHCPILKQGP
QDPQELQCIKIAQAGEDPHGYFLPGQFAFSRALRTK.

Mature mouse chemerin has the amino acid sequence:

(SEQ ID NO: 6)
TEPELSETQRRSLQVALEEFHKHPPVQLAFQEIGVDRAEEVL
FSAGTFVRLEFKLQQTNCPKKDWKKPECTIKPNGRRRKCLAC
IKMDPKGKILGRIVHCPILKQGPQDPQELQCIKIAQAGEDPH
GYFLPGQFAFSRALRTK.

Rat prochemerin has the amino acid sequence:

(SEQ ID NO: 7)
TELELSETQRRGLQVALEEFHRHPPVQWAFQEIGVDSADDLF
FSAGTFVRLEFKLQQTSCLKKDWKKPECTIKPNGRKRKCLAC
IKLDPKGKVLGRMVHCPILKQGPQQEPQESQCSKIAQAGEDS
RIYFFPGQFAFSRALQSK.

Mature mouse chemerin has the amino acid sequence:

(SEQ ID NO: 8)
MKCLLISLALWLGTADIHGTELELSETQRRGLQVALEEFHRHP
PVQWAFQEIGVDSADDLFFSAGTFVRLEFKLQQTSCLKKDWKK
PECTIKPNGRKRKCLACIKLDPKGKVLGRMVHCPILKQGPQQE
PQESQCSKIAQAGEDSRIYFFPGQFAFSRALQSK.

Chemerin is a potent macrophage chemoattractant the acts via the G protein-coupled receptor ChemR23. Proteolyzed compositions of mouse chemerin inhibit macrophage activation and inhibition of inflammation in the presence of the Chem23 receptor. A 15 amino acid C-terminal peptide (mC15) of mouse chemerin (AGEDPHGYFLPGQFA (SEQ ID NO: 9)) inhibits activation of macrophages and in the presence of ChemR23. As shown in the data provided herein, human chemerin C15 peptides (e.g. AGEDPHSFYFPGQFA (SEQ ID NO: 1)) also are potent inflammatory inhibitors.

Accordingly, disclosed herein, in certain embodiments, are methods of modulating the activity of cells expressing the chemerin GPCR receptor, ChemR23. In some embodiments these cells are antigen presenting cells. In some embodiments, these cells include macrophages, dendritic cells, monocytes, neutrophils and NK cells among others which are a source of cytokines linked to inflammatory skin disorders. In some embodiments, the chemerin C15 peptides act to reduce the secretion of cytokines by the ChemR23 expressing cells. In some embodiments, the chemerin C15 peptides decrease release of inflammatory cytokines such as IL-23, TNFα, IL-1β, IL-6, and RANTES. In some embodiments, the chemerin C15 peptides decrease release of IL-23. In some embodiments, the chemerin C15 peptides decrease release of TNFα. In some embodiments, the chemerin C15 peptides decrease release of IL-1β. In some embodiments, the chemerin C15 peptides decrease release of IL-6. In some embodiments, the chemerin C15 peptides decrease release of RANTES. In some embodiments, the chemerin C15 peptides prevent the recruitment of inflammatory immune cells. In some embodiments, the chemerin C15 peptides inhibit the transcription of inflammatory cytokines such as IL-23, TNFα, IL-1β, IL-6, and RANTES. In some embodiments, the chemerin C15 peptides inhibit the transcription of IL-23. In some embodiments, the chemerin C15 peptides inhibit the transcription of TNFα. In some embodiments, the chemerin C15 peptides inhibit the transcription of IL-1β. In some embodiments, the chemerin C15 peptides inhibit the transcription of IL-6. In some embodiments, the chemerin C15 peptides inhibit the transcription of RANTES. In some embodiments, the chemerin C15 peptides prevent the recruitment of inflammatory immune cells. In some embodiments, the chemerin C15 peptides prevent the activation of inflammatory immune cells. In some embodiments, the chemerin C15 peptides inhibit the activation of T cells.

As shown in the data provided herein, chemerin C15 peptides are not direct competitive inhibitors of chemerin binding to ChemR23. The chemerin C15 peptides thus exhibit properties of a dominant negative inhibitor, a biased ligand, or an allosteric antagonist. As such, they are capable of beneficially blocking inflammatory signals (e.g., cytokine release) via Chemerin/ChemR23 signaling and/or the signaling associated with accessory proteins to ChemR23 without inhibiting ‘normal’ Chemerin/ChemR23 and/or the signaling associated with accessory proteins to ChemR23 which lead to ‘side effects’. Furthermore, the C15 peptides inhibit inflammatory processes stimulated by TNFα, IFNγ, LPS, Zymosan and other stimuli which do not signal directly through ChemR23. In this manner, the C15 peptides exhibit properties of an inhibitor of the NFκB pathway. As such, they are capable of beneficially blocking inflammatory signals (e.g., cytokine release) via prevention of NFκB activation, nuclear translocation, cytokine gene transcription and/or cytokine release without exhibiting adrenosuppression or other side effects associated with corticosteroids.

In addition, as shown in the data provided herein, chemerin C15 peptides contain an FYFP motif (SEQ ID NO: 2) and lose the ability to inhibit inflammatory cytokine production in stimulated macrophages if the peptide is modified in the FYFP motif (SEQ ID NO: 2) to FYAP (SEQ ID NO: 10) or YFAP (SEQ ID NO: 11). In human C15, the FYFP motif (SEQ ID NO: 2) is embodied in its exact FYFP sequence (SEQ ID NO: 2), while in murine C15, the FYFP motif (SEQ ID NO: 2) is embodied in the YFLP amino acid sequence (SEQ ID NO: 12). The FYFP motif (SEQ ID NO: 2) is similar to the conserved FYFP motif (SEQ ID NO: 2) of the PP2A regulatory B-subunit. Binding the B-subunit to PP2A core enzyme A and C subunits is dependent on the FYFP motif (SEQ ID NO: 2) (Davis A J, et al. J Biol Chem. 2008; 283:16104-14). Under resting conditions, the protein phosphatase 2A (PP2A) core enzyme associates with IKK (IκB Kinase), the kinase which phosphorylates IκB and maintains it in an inactive unphosphorylated state. Additionally, PP2A core associates with NFκB of the NFκB/IκB complex, maintaining it in a resting unphosphorylated state. During activation of the NFκB pathway, NFκB and IκB are phosphorylated and PP2A association with the NFκB/IkB is diminished by association with the PP2A regulatory B-subunit. IκB also is released, thus allowing NFκB to translocate to the nucleus where it participates in cytokine transcription, including induction of IL-23 transcription. In some embodiments, binding of chemerin C15 peptide to PP2A interferes with binding of the regulatory B-subunit to the complex and thus stabilizes the NFκB/IκB in a resting state. In some embodiments, chemerin C15 peptides inhibit cytokine production by inhibiting the release of IκB from the NFκB, which prevents nuclear translocation and gene activation.

Described herein, in certain embodiments, are topical formulations comprising a chemerin C15 peptide for treating a inflammatory dermatological disorder. In some embodiments, the inflammatory dermatological disorder is a chronic blistering disorder, acne, psoriasis, dermatitis (e.g., contact or atopic), eczema, lichen planus, alopecia greata, urticaria, rosacea, scarring (i.e. the formation of a scar (e.g., a keloid scar or a hypertrophic scar)), and/or melanoma. In some embodiments, the inflammatory dermatological disorder is psoriasis. In some embodiments, the inflammatory dermatological disorder is dermatitis. In some embodiments, the inflammatory dermatological disorder is atopic dermatitis. In some embodiments, the inflammatory dermatological disorder is contact dermatitis. In some embodiments, a topical formulation disclosed herein comprises a therapeutically-effective amount of a chemerin C15 peptide. The topical formulations provided herein deliver therapeutic levels of the chemerin C15 peptide beneath the stratum corneum to the epidermis and dermis and offer an enhanced treatment of skin disorders, particularly inflammatory skin disorders.

Also described herein are methods for the administration of topical formulations comprising a chemerin C15 peptide. In one aspect, topical administration of a chemerin C15 peptide provides for local treatment of dermatological conditions. In one aspect, local treatment of dermal conditions with a chemerin C15 peptide reduces possible side effects associated with systemic administration of a chemerin C15 peptide. In one aspect, topical administration of a chemerin C15 peptide to a mammal minimizes systemic absorption of the chemerin C15 peptide. In some embodiments, a topical formulation disclosed herein is administered before or after contact with an allergen and/or irritant.

In certain embodiments, chemerin C15 peptides applied locally for a skin disorder will have fewer or less severe side effect than currently approved topical agents for the treatment of skin disorders. These approved topical agents include steroids (e.g., corticosteroids) and calcineurin antagonists (e.g., Elidel) which carry known risks of thinning of the skin, cataracts, glaucoma and/or neoplasms when used topically in the treatment of skin disorders. In certain embodiments, a chemerin C15 peptide applied locally for a skin disorder is a naturally occurring biological agent with fewer or less severe side effect than currently approved systemic biological agents for the treatment of skin disorders. These approved systemic biological agents include mono-clonal antibodies (e.g., Stelara) and fusion proteins (e.g., Enbrel) which carry known risks of antigenic response, infections and malignancies.

In certain embodiments, chemerin C15 peptides are formulated for topical administration to minimize systemic exposure of the chemerin C15 peptides. In certain embodiments, topical formulations of chemerin C15 peptides are designed to minimize systemic exposure of the chemerin C15 peptides (e.g., certain excipients are excluded which may result in the chemerin C15 peptides penetrating the skin and becoming systemically available). In some embodiments, minimizeing systemic exposure reduces unwanted side-effects (e.g., effects on non-targeted parts of the body) of administering a chemerin C15 peptide.

Disclosed herein is the use of chemerin C15 peptides in the manufacture of medicaments suitable for topical administration to a mammal for the treatment or prevention of dermatological diseases, disorders or conditions.

Described herein are pharmaceutical compositions suitable for topical administration, methods for treating, methods for formulating topical formulations, methods for producing, methods for manufacturing, treatment strategies, using chemerin C15 peptides.

### Chemerin C15 Peptides

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

The chemerin C15 peptides provided herein for administration exhibit one or more properties or activities useful as a topical treatment for an inflammatory disease or disorder. In some embodiments, a chemerin C15 peptide disclosed herein inhibits inflammation. In some embodiments, a chemerin C15 peptide disclosed herein inhibits inflammation associated with a dermatological disease or disorder. In some embodiments, a chemerin C15 peptide disclosed herein inhibits one or more cellular processes associated with inflammation. In some embodiments, a chemerin C15 peptide disclosed herein inhibits the release of one or more inflammatory cytokines. Exemplary inflammatory cytokine include, but are not limited to, IL-23, IL-12, TNFα, IL-1β, IL-6, or RANTES. In some embodiments, a chemerin C15 peptide disclosed herein inhibits the release of IL-23, IL-12, TNFα, IL-1β, IL-6, or RANTES. In some embodiments, a chemerin C15 peptide disclosed herein inhibits the transcription of one or more inflammatory cytokines. In some embodiments, a chemerin C15 peptide disclosed herein inhibits the transcription of IL-23, IL-12, TNFα, IL-1β, IL-6, or RANTES. In some embodiments, a chemerin C15 peptide disclosed herein inhibits the production of one or more inflammatory cytokines. In some embodiments, a chemerin C15 peptide disclosed herein inhibits the production and/or release of IL-23, IL-12, TNFα, IL-1β, IL-6, or RANTES. In some embodiments, a chemerin C15 peptide disclosed herein inhibits the production and/or release of one or more inflammatory cytokines by immune cells. In some embodiments, a chemerin C15 peptide disclosed herein inhibits the production and/or release of IL-23, IL-12, TNFα, IL-1β, IL-6, or RANTES by immune cells. In some embodiments, a chemerin C15 peptide disclosed herein inhibits the production and/or release of one or more inflammatory cytokines by antigen presenting cells. In some embodiments, a chemerin C15 peptide disclosed herein inhibits the production and/or release of IL-23, IL-12, TNFα, IL-1β, IL-6, or RANTES by antigen presenting cells. In some embodiments, a chemerin C15 peptide disclosed herein inhibits the production and/or release of one or more inflammatory cytokines in myeloid dendritic cells (mDC), plasmacytoid dendritic cells (pDC) or macrophages. In some embodiments, a chemerin C15 peptide disclosed herein inhibits the production and/or release of IL-23, IL-12, TNFα, IL-1β, IL-6, or RANTES in myeloid dendritic cells (mDC), plasmacytoid dendritic cells (pDC) or macrophages. In some embodiments, a chemerin C15 peptide disclosed herein inhibits the production and/or release of one or more inflammatory cytokines by immune cells expressing the ChemR23 receptor. In some embodiments, a chemerin C15 peptide disclosed herein inhibits the production and/or release of IL-23, IL-12, TNFα, IL-1β, IL-6, or RANTES by immune cells expressing the ChemR23 receptor.

In some embodiments, a chemerin C15 peptide disclosed herein inhibits the activation of NF-κB. In some embodiments, a chemerin C15 peptide disclosed herein inhibits the activation of NF-κB associated with inflammation. In some embodiments, a chemerin C15 peptide disclosed herein inhibits the activation of NF-κB in cells expressing the ChemR23 receptor. In some embodiments, a chemerin C15 peptide disclosed herein binds to the protein phosphatase 2A core enzyme. In some embodiments, a chemerin C15 peptide disclosed herein prevents the release of IκB from NF-κB. In some embodiments, a chemerin C15 peptide disclosed herein prevents the nuclear translocation of NF-κB. In some embodiments, a chemerin C15 peptide disclosed herein inhibits Th1 and/or Th17 T-cell activation. In some embodiments, a chemerin C15 peptide disclosed herein inhibits Th1 and/or Th17 T-cell activation associated with inflammation.

In some embodiments, the chemerin C15 peptide is any suitable chemerin C15 peptide for topical administration. In some embodiments, the chemerin C15 peptide is human chemerin C15 peptide. In some embodiments, the chemerin C15 peptide comprises a sequence of amino acids AGEDPHSFYFPGQFA (SEQ ID NO: 1). In some embodiments, the chemerin C15 peptide has a sequence of amino acids consists essentially of the sequence of amino acids AGEDPHSFYFPGQFA (SEQ ID NO: 1).

In some embodiments, the chemerin C15 peptide is a mouse chemerin C15 peptide. In some embodiments, the chemerin C15 peptide comprises a sequence of amino acids AGEDPHGYFLPGQFA (SEQ ID NO: 9). In some embodiments, the chemerin C15 peptide has a sequence of amino acids consists essentially of the sequence of amino acids AGEDPHGYFLPGQFA (SEQ ID NO: 9).

In some embodiments, the chemerin C15 peptide is a chimeric chemerin C15 peptide comprising a sequence of amino acids derived from a human chemerin C15 peptide and a non-human chemerin C15 peptide. In some embodiments, the chemerin C15 peptide is a chimeric chemerin C15 peptide comprising a sequence of amino acids derived from a human chemerin C15 peptide and a mouse chemerin C15 peptide. In some embodiments, the chemerin C15 peptide comprises a sequence of amino acids AGEDPHGYYFPGQFA (SEQ ID NO: 13). In some embodiments, the chemerin C15 peptide has a sequence of amino acids consists essentially of the sequence of amino acids AGEDPHGYYFPGQFA (SEQ ID NO: 13).

In some embodiments, the chemerin C15 peptide is a peptide comprising the sequence of amino acids AGEDPHSX₁X₂X₃PGQFA (SEQ ID NO: 14), where X₁, X₂, and X₃ are hydrophobic amino acids. In some embodiments, the chemerin C15 peptide is a peptide comprising the sequence of amino acids AGEDPHSX₁X₂X₃PGQFA (SEQ ID NO: 15), where X₁, X₂, and X₃ are aromatic amino acids. In some embodiments, X₁ is tyrosine or phenyalanine. In some embodiments, X₂ is tyrosine or phenyalanine. In some embodiments, X₂ is tyrosine or phenyalanine.

In some embodiments, the chemerin C15 peptide comprises a sequence of amino acids derived from a chemerin C15 peptide and a regulatory B-subunit of PP2A. In some embodiments, the chemerin C15 peptide comprises a sequence of amino acids derived from a human chemerin C15 peptide and a human regulatory B-subunit of PP2A. In some embodiments, the chemerin C15 peptide comprises a sequence of amino acids PTFYFP (SEQ ID NO: 16). In some embodiments, the chemerin C15 peptide comprises a sequence of amino acids AGEDPTFYFPGQFA (SEQ ID NO: 17). In some embodiments, the chemerin C15 peptide consists essentially of a sequence of amino acids AGEDPTFYFPGQFA (SEQ ID NO: 17).

In some embodiments, the chemerin C15 peptide comprises the amino acid sequence AGEDPHSFYFPGQFA (SEQ ID NO: 1), where one or more amino acids of the sequence AGEDPHSFYFPGQFA (SEQ ID NO: 1) is substituted. In some embodiments, 1, 2, 3, 4, 5, 6, 7, 8, 9, 10, 11, 12, 13, 14 or 15 amino acids are substituted.

In some embodiments, the chemerin C15 peptide comprises the amino acid sequence AGEDPHSFYFPGQFA (SEQ ID NO: 1), where one or more amino acids in the sequence PHSFYFP (SEQ ID NO: 18) is substituted. In some embodiments, 1, 2, 3, 4, 5, 6, or 7 amino acids are substituted.

In some embodiments, the chemerin C15 peptide comprises L-amino acids. In some embodiments, the chemerin C15 peptide comprises a sequence of amino acids AGEDPHSFYFPGQFA (SEQ ID NO: 1), where the peptide comprises L-amino acids. In some embodiments, the chemerin C15 peptide has a sequence of amino acids consists essentially of the sequence of amino acids AGEDPHSFYFPGQFA (SEQ ID NO: 1), where the peptide comprises L-amino acids.

In some embodiments, the chemerin C15 peptide comprises D- and/or L-amino acids. In some embodiments, the chemerin C15 peptide comprises a sequence of amino acids AGEDPHSFYFPGQFA (SEQ ID NO: 1), where the peptide comprises D- and/or L-amino acids. In some embodiments, the chemerin C15 peptide has a sequence of amino acids consists essentially of the sequence of amino acids AGEDPHSFYFPGQFA (SEQ ID NO: 1), where the peptide comprises D- and/or L-amino acids.

In some embodiments, the chemerin C15 peptide comprises a sequence of amino acids AGEDPHSFYFPGQFA (SEQ ID NO: 1), where one or more amino acids of the sequence AGEDPHSFYFPGQFA (SEQ ID NO: 1) is in the D-configuration. In some embodiments, the chemerin C15 peptide comprises a sequence of amino acids AGEDPHSFYFPGQFA (SEQ ID NO: 1), where each amino of the sequence AGEDPHSFYFPGQFA (SEQ ID NO: 1) is in the D-configuration. In such examples, the sequence where each amino of the sequence is in the D-configuration is called a retroinverso peptide sequence. In such examples, the chemerin C15 peptide comprises a sequence of amino acids AFQGPFYFSHPDEGA (SEQ ID NO: 19).

In some embodiments, the chemerin C15 peptide comprises a sequence of amino acids comprising retroinverso sequences representing chemerin C-terminal fragments of human chemerin sequences (e.g., AGEDPHSFYFPGQFA (SEQ ID NO: 1). In some embodiments, the chemerin C15 peptide comprises a sequence of amino acids comprising retroinverso sequences representing chemerin C-terminal fragments of non-human chemerin sequences, such as for example, mouse chemerin C15 peptide (e.g., AGEDPHGYFLPGQFA (SEQ ID NO: 9)).

In some embodiments, the chemerin C15 peptide comprises derivatives or analogs in which a substituted amino acid residue is not one encoded by the genetic code (i.e. an unnatural amino acid). In some embodiments, the chemerin C15 peptide comprises one or more unnatural amino acids. In some embodiments, the chemerin C15 peptide comprises a sequence of amino acids AGEDPHSFYFPGQFA (SEQ ID NO: 1), where one or more amino acids is a unnatural amino acid. In some embodiments, the chemerin C15 peptide has a sequence of amino acids consists essentially of the sequence of amino acids AGEDPHSFYFPGQFA (SEQ ID NO: 1), where one or more amino acids is a unnatural amino acid.

Examples of unnatural amino acids that can be incorporated into the chemerin C15 peptide provided include, but are not limited to, homoserine (hSer), homoserine lactone (hSerlac), homocysteine (Hcy), homoarginine (hArg), homocitrulline (Hci), penicillamine (Pen), Nα-methylarginine (N-MeArg), norleucine (Nle), norvaline (Nval), norisoleucine (NIle), N-methylisoleucine (N-MeIle), phenylglycine (PhG), t-butylglycine (Tle), hydroxyproline (Hyp), 3,4-dehydroproline (Δ-Pro), pyroglutamine (Pyr,Glp), ornithine (Orn), 1-aminoisobutyric acid (1-Aib), 2-aminoisobutyric acid (2-Aib), 2-aminobutyric acid (2-Abu), 4-aminobutyric acid (4-Abu), 2,4-diaminobutyric acid (A2bu), α-aminosuberic acid (Asu), albizzin (Abz), β-cyclohexylalanine (Cha), 3-(1-naphthyl)alanine (1-NaI), 3-(2-naphthyl)alanine (2-NaI), citrulline (Cit). pipecolinic acid (Pip), 4-chlorophenylalanine (4-ClPhe), 4-fluorophenylalanine (4-FPhe), sarcosine (Sar) and 1-aminopropanecarboxylic acid (1-NCPC). Additional unnatural amino acid include, but are not limited to those disclosed in U.S. Patent Application Pub. No. 2004/0121438 and U.S. Pat. No. 5,656,727. Both natural and unnatural amino acids are commercially available from vendors such as NovaBiochem (San Diego, Calif., USA) and Bachem (Torrance, Calif., USA).

In some embodiments, the chemerin C15 peptide comprises a sequence of amino acids AGEDPHSFYFPGQFA (SEQ ID NO: 1), where 1, 2, 3, 4, 5, 6, 7, 8, 9, 10, 11, 12, 13, 14, or 15 amino acids is a unnatural amino acid. In some embodiments, the chemerin C15 peptide has a sequence of amino acids consists essentially of the sequence of amino acids AGEDPHSFYFPGQFA (SEQ ID NO: 1), where 1, 2, 3, 4, 5, 6, 7, 8, 9, 10, 11, 12, 13, 14, or 15 amino acids is a unnatural amino acid.

In some embodiments, the chemerin C15 peptide comprises a sequence of amino acids AGEDPHX₁FYFPGQFA (SEQ ID NO: 20), where X₁ is a unnatural amino acid. In some embodiments, the chemerin C15 peptide has a sequence of amino acids consists essentially of the sequence of amino acids AGEDPHX₁FYFPGQFA (SEQ ID NO: 20), where X₁ is a unnatural amino acid. In some embodiments, X₁ is a derivative of the amino acid serine. In some embodiments, X₁ is homoserine.

In some embodiments, the chemerin C15 peptide comprises a sequence of amino acids AGEDPHSX₁YFPGQFA (SEQ ID NO: 21), where X₁ is a unnatural amino acid. In some embodiments, the chemerin C15 peptide has a sequence of amino acids consists essentially of the sequence of amino acids AGEDPHSX₁YFPGQFA (SEQ ID NO: 21), where X₁ is a unnatural amino acid. In some embodiments, X₁ is a derivative of the amino acid phenylalanine or tyrosine. In some embodiments, X₁ is p-chlorophenylalanine. In some embodiments, X₁ is napthyl alanine.

In some embodiments, the chemerin C15 peptide comprises a sequence of amino acids AGEDPHSX₁YX₂PGQFA (SEQ ID NO: 22), where X₁ and X₂ are unnatural amino acids. In some embodiments, X₁ and X₂ are the same unnatural amino acid. In some embodiments, X₁ and X₂ are different unnatural amino acids. In some embodiments, the chemerin C15 peptide has a sequence of amino acids consists essentially of the sequence of amino acids AGEDPHSX₁YX₂PGQFA, where X₁ and X₂ are unnatural amino acids (SEQ ID NO: 22). In some embodiments, X₁ and X₂ are the same unnatural amino acid. In some embodiments, X₁ and X₂ are different unnatural amino acids. In some embodiments, X₁ is an aromatic unnatural amino acid. In some embodiments, X₁ is a derivative of the amino acid phenylalanine or tyrosine. In some embodiments, X₁ is p-chlorophenylalanine. In some embodiments, X₁ is napthyl alanine. In some embodiments, X₂ is an aromatic unnatural amino acid. In some embodiments, X₂ is p-chlorophenylalanine. In some embodiments, X₂ is napthyl alanine.

In some embodiments, the chemerin C15 peptide comprises a sequence of amino acids AGEDPHSX₁X₂X₃PGQFA (SEQ ID NO: 23), where X₁, X₂ and X₃ are unnatural amino acids. In some embodiments, X₁ and X₂ are the same unnatural amino acid. In some embodiments, X₁ and X₂ are different unnatural amino acids. In some embodiments, X₁ and X₃ are the same unnatural amino acid. In some embodiments, X₁ and X₃ are different unnatural amino acids. In some embodiments, X₂ and X₃ are the same unnatural amino acid. In some embodiments, X₂ and X₃ are different unnatural amino acids. In some embodiments, X₁, X₂ and X₃ are the same unnatural amino acid. In some embodiments, X₁, X₂ and X₃ are different unnatural amino acids. In some embodiments, X₁ is an aromatic unnatural amino acid. In some embodiments, X₁ is a derivative of the amino acid phenylalanine or tyrosine. In some embodiments, X₁ is p-chlorophenylalanine. In some embodiments, X₁ is napthyl alanine. In some embodiments, X₂ is an aromatic unnatural amino acid. In some embodiments, X₂ is a derivative of the amino acid phenylalanine or tyrosine. In some embodiments, X₂ is p-chlorophenylalanine. In some embodiments, X₂ is napthyl alanine. In some embodiments, X₃ is an aromatic unnatural amino acid. In some embodiments, X₃ is a derivative of the amino acid phenylalanine or tyrosine. In some embodiments, X₃ is p-chlorophenylalanine. In some embodiments, X₃ is napthyl alanine.

In some embodiments, unnatural amino acids are selected from commercially available amino acids. In some embodiments, unnatural amino acids are selected from D-configuration, L-configuration or achiral amino acids which do not occur in nature (e.g. listed in the Accelrys Available Chemicals Directory (ACD), http://accelrys.com). In some embodiments, unnatural amino acids are selected for improvements to solubility, stability, potency, mechanism of action, and/or pharmaceutical properties of the peptide.

In some embodiments, the chemerin C15 peptide comprises a sequence of amino acids comprising chimeric sequences and retroinverso sequences containing one or more unnatural amino acids selected from commercially available unnatural amino acids (e.g. listed in the Accelrys Available Chemicals Directory (ACD), http://accelrys.com) and selected for improvements to solubility, stability, potency, mechanism of action, pharmaceutical properties of the peptide.

In some embodiments, the chemerin C15 peptide exhibits increased inhibition of cytokine production in stimulated macrophages compared to a human chemerin C16 peptide having the sequence of amino acids AGEDPHSFYFPGQFAF (SEQ ID NO: 24). In some embodiments, the chemerin C15 peptide exhibits increased inhibition of IL-23 production in stimulated macrophages compared to a human chemerin C16 peptide having the sequence of amino acids AGEDPHSFYFPGQFAF (SEQ ID NO: 24).

In some embodiments, the chemerin C15 peptide exhibits increased inhibition of cytokine production in stimulated macrophages compared to a human chemerin C17 peptide having the sequence of amino acids AGEDPHSFYFPGQFAFS (SEQ ID NO: 25). In some embodiments, the chemerin C15 peptide exhibits increased inhibition of IL-23 production in stimulated macrophages compared to a human chemerin C17 peptide having the sequence of amino acids AGEDPHSFYFPGQFAFS (SEQ ID NO: 25).

In some embodiments, the chemerin C15 peptide exhibits increased inhibition of cytokine production in stimulated macrophages compared to a mouse chemerin C15 peptide having the sequence of amino acids AGEDPHGYFLPGQFA (SEQ ID NO: 9). In some embodiments, the chemerin C15 peptide exhibits increased inhibition of IL-23 production in stimulated macrophages compared to a mouse chemerin C15 peptide having the sequence of amino acids AGEDPHGYFLPGQFA (SEQ ID NO: 9).

In some embodiments, the chemerin C15 peptide does not exhibit agonist activity toward the Chem23 receptor.

In some embodiments, the chemerin C15 peptide is a peptide salt such as pharmaceutically acceptable acid- or base addition salt. Salts of peptides or functional equivalents are prepared by known methods, which typically involve the mixing of the peptide with either a pharmaceutically acceptable acid to form an acid addition salt, or with a pharmaceutically acceptable base to form a base addition salt. Whether an acid or a base is pharmaceutically acceptable can be easily decided by a person skilled in the art after taking the specific intended use of the compound into consideration. Depending on the intended use, pharmaceutically acceptable acids include organic and inorganic acids such as formic acid, acetic acid, propionic acid, lactic acid, glycolic acid, oxalic acid, pyruvic acid, succinic acid, maleic acid, malonic acid, cinnamic acid, sulphuric acid, hydrochloric acid, hydrobromic acid, nitric acid, perchloric acid, phosphoric acid, and thiocyanic acid, which form ammonium salts with free amino groups of peptides and functional equivalents. Pharmaceutically acceptable bases, which form carboxylate salts with free carboxylic groups of peptides and functional equivalents, include ethylamine, methylamine, dimethylamine, triethylamine, isopropylamine, diisopropylamine, and other mono-, di- and trialkylamines, as well as arylamines. Moreover, also pharmaceutically acceptable solvates, complexes or adducts, such as hydrates or ethurates, alkali metal salt, such as lithium, sodium or potassium salts, or other salts such as, but not limited to calcium magnesium aluminum, zinc or iron salts, are encompassed.

In some embodiments, the chemerin C15 peptide is a multimer comprising one or more chemerin C15 peptides.

### Peptide Modifications

In some embodiments, the chemerin C15 peptide is further modified to improve one or more properties of the chemerin C15 peptide. Exemplary properties include, but are not limited to, solubility, stability, potency, mechanism of action, ability to be detected and/or pharmaceutical properties of the chemerin C15 peptide. Generally, the modifications do not significantly reduce the therapeutic properties of the chemerin C15 peptide, such as the anti-inflammatory properties of the chemerin C15 peptide, including, for example, inhibition of NFκB and secretion and/or production of one or more inflammatory cytokines (e.g. IL-23, IL-12, TNFα, IL-1β, IL-6, or RANTES).

In some embodiments, the chemerin C15 peptide is further modified by natural processes, such as processing and other known post-translational modifications, or by chemical or enzymatic techniques well-known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cysteine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

In some embodiments, the modification increases the solubility of the chemerin C15 peptide. In one example, amidation increases the solubility of the chemerin C15 peptide. In some embodiments, the modification renders that the chemerin C15 peptide less susceptible to protease degradation. In some embodiments, the modification increases the ability of the chemerin C15 peptide to penetrate the skin. In one example, lipidation increases the ability of the chemerin C15 peptide to penetrate the skin. In some embodiments, a hydrogen of the N-terminal amino group of the peptide is replaced. In some embodiments, the entire N-terminal amino group of the peptide is replaced. In some embodiments, the hydroxyl group (OH) of the C-terminal carboxylic group is replaced. In some embodiments, the entire C-terminal carboxylic group is replaced.

In some embodiments, functional groups of the chemerin C15 peptide that are modified include hydroxyl, amino, guanidinium, carboxyl, amide, phenol, imidizole rings or sulfhydryl. Exemplary non-limiting reaction of such groups, include acetylation of hydroxyl groups by alkyl halides; esterification, amidation or hydrogenization (i.e. reduction to alcohol) of carboxyl groups; deamidation, acylation, alkylation, arylation of amino groups (e.g. primary amino group of the peptide or the amino group of lysine residues); halogenation or nitration of tyrosine phenol groups.

Modification of peptides are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins-Structure & Molecular Properties (2nd ed., T. E. Creighton, W.H. Freeman & Co., NY, 1993). Many detailed reviews are available on this subject, such as by Wold, Posttranslational Covalent Modification of Proteins, 1-12 (Johnson, ed., Acad. Press, NY, 1983); Seifter et al., 182 Meth. Enzymol. 626-46 (1990); and Rattan et al., 663 Ann N.Y. Acad. Sci. 48-62 (1992).

In some embodiments, the chemerin C15 peptide is conjugated to soluble or insoluble carrier molecule to modify their solubility properties as needed and to increase the local concentrations of peptides in targeted tissues. Examples of soluble carrier molecules include, but are not limited to, polymers of polyethyleneglycol (PEG) and polyvinylpyrrolidone; examples of insoluble polymers include silicates, polystyrene, and cellulose.

In some embodiments, the chemerin C15 peptides are micro-encapsulated to enhance their stability during and after therapeutic application. In some embodiments, polyester or PEG microspheres are used to encapsulate and stabilize the chemerin C15 peptides. Various methods of preparing microspheres for peptide encapsulation are known in art. The method selected depends upon the hydrophilic or hydrophobic nature of the peptide composition to be encapsulated. Examples of protocols for such methods are found in Wang H T et al. (1991, J. Control. Release 17:23-25) and U.S. Pat. No. 4,324,683, both of which are incorporated herein in their entirety. In some embodiments, in vitro peptide release studies are performed to determine the relative availability of the peptide after it has been incorporated into a microsphere. In an exemplary method, microspheres (about 200 mg) are suspended in pH 7.2 phosphate-buffered saline (PBS) (2.5 ml) and agitated at 37° C. and 100 rpm in an environmental incubator shaker (G-24, New Brunswick Scientific Co., Edison, N.J.). At specific sampling times (each day for the first 4 days and every other day thereafter), the buffer solution is completely removed and replaced with fresh PBS. The peptide content of the PBS is measured using the Bradford method or other suitable quantitative assay typically used for protein analysis.

In some embodiments, the chemerin C15 peptide is further modified by attachment of detectable moiety, such for example, a fluorescent dye or a radiolabeled moiety. Exemplary detectable moieties are known in the art and include, but are not limited to, Rhodamine, Fluorescein, Cy3, Alexa Fluor 405, Alexa Fluor 488, Alexa Fluor 546, Alexa Fluor 555, Alexa Fluor 633, Alexa Fluor 647, Allophycocyanin (APC), APC-Cy7, fluorescein isothiocyanate (FITC), Pacific Blue, R-phycoerythrin (R-PE), PE-Cy5, PE-Cy7, Texas Red, PE-Texas Red, peridinin chlorophyll protein (PerCP), PerCP-Cy5.5.

In some embodiments, the peptide is conjugated to an immunogenic carrier peptide. In some embodiments, conjugation to an immunogenic carrier peptide allows for the production of C15 peptide specific antibodies. In some embodiments, the immunogenic peptide is Keyhole limpet hemocyanin (KLH).

### Production of Chemerin C15 Peptides

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

The chemerin C15 peptides provided herein can be produced using any method known to those skilled in the art. In some embodiments, the peptides are produced using recombinant methods of expressing peptides in cells or in animals. In some embodiments, the peptides are produced in vitro using chemical synthesis.

In some examples, the chemerin C15 peptides are generated by protease cleavage of a chemerin polypeptide. In some embodiments, the chemerin C15 peptides are generated by an in vitro protease reaction where a chemerin polypeptide is incubated with a cysteine protease that cleaves the C-terminal end of the polypeptide to produce the 15 amino acid length chemerin C15 peptide. In some embodiments, the chemerin polypeptide employed in the reaction is a native protein. In some embodiments, the chemerin polypeptide employed in the reaction is a recombinant protein. In some embodiments, the chemerin C15 peptide is purified from the reaction by a suitable purification method, such as for example, HPLC or dialysis. In some embodiments, the purified chemerin C15 peptide is further modified as described elsewhere herein.

In some embodiments, the peptides are produced using chemical synthesis methods known to those skilled in the art such as those disclosed in Merrifield, R. B., Solid Phase Peptide Synthesis I., J. Am. Chem. Soc. 85:2149-2154 (1963); Camino, L. A. et al., [(9-Fluorenylmethyl)Oxy]Carbonyl (Fmoc) Amino Acid Chlorides: Synthesis, Characterization, And Application To The Rapid Synthesis Of Short Peptides, J. Org. Chem. 37:51:3732-3734; Merrifield, R. B. et al., Instrument For Automated Synthesis Of Peptides, Anal. Chem. 38:1905-1914 (1966); or Kent, S. B. H. et al., High Yield Chemical Synthesis Of Biologically Active Peptides On An Automated Peptide Synthesizer Of Novel Design, IN: Peptides 1984 (Ragnarsson U., ed.) Almqvist and Wiksell Int., Stockholm (Sweden), pp. 185-188, all of which are incorporated by reference herein in their entirety. In some embodiments, the peptides are produced by a machine capable of sequential addition of amino acids to a growing peptide chain. In some embodiments, the peptides are manufactured using standard solution phase methodology, which can be amenable to large-scale production efforts. In an exemplary method, the peptides are generated using solid phase synthesis by addition of FMOC-protected amino acids followed by final cleavage of the peptide using the trifluoroacetic acid (TFA). In some embodiments, the peptide is then purified. In some embodiments, the peptide is purified by HPLC purification. In some embodiments, the peptide is purified by HPLC purification on a C18 column with a gradient of water/acetronitrile.

### Dermatological Disorders (Dermatoses)

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

As used herein, an inflammatory dermatological disorder includes a dermatological disorder is caused by (either partially or fully) an immune disorder, (e.g. an autoimmune disorder (e.g., eczema, psoriasis)); a proliferation disorder (e.g., melanoma); contact with an allergen and/or an irritant; an overproduction of sebum lipids (e.g., acne); a fibroblast disorder (e.g., scarring after a trauma (e.g., surgery)); or combinations thereof. Dermatological disorders include, but are not limited to, psoriasis, atopic dermatitis, irritant contact dermatitis, eczematous dermatitis, a chronic blistering (bullous) disorder, acne, seborrhoeic cutaneous manifestations of immunologically-mediated disorders, alopecia, alopecia greata, adult respiratory distress syndrome, pulmonary fibrosis, scleredoma, scar formation, (e.g., a keloid scar or a hypertrophic scar), urticaria, rosacea, melanoma, chronic obstructive pulmonary disease (COPD), inflammation from kidney transplant, asthma, hidradentis supporativa, rheumatoid arthritis, psoriatic arthritis, Sjogren's Syndrome, uveitis, Graft vs. Host disease (GVHD), Oral Lichen Planus, arthralgia or Islet Cell Transplant inflammation. In some embodiments, the dermatological disorder is psoriasis. In some embodiments, the dermatological disorder is dermatitis. In some embodiments, the dermatological disorder is atopic dermatitis. In some embodiments, the dermatological disorder is contact dermatitis.

### Psoriasis

Disclosed herein are methods of treating psoriasis in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

In certain instances, the symptoms of psoriasis result from (either partially or fully) the exudation of plasma from vessels and capillaries into the epidermis, dermis, and/or subcutaneous tissues. T helper (Th) 17 cells are involved in the pathogenesis of psoriasis and other autoimmune inflammatory diseases. Interleukin (IL)-23 stimulates survival and proliferation of Th17 cells, and thus serves major cytokine regulator for these diseases. In psoriasis, IL-23 is overproduced by dendritic cells and keratinocytes. IL-23 stimulates Th17 cells within dermis to make IL-17A and IL-22. IL-22, in particular, drives keratinocyte hyperproliferation in psoriasis (Fitch et al. (2007) Curr Rheumatol Rep. 9(6):461-7). Interleukin-12/23p40 and TNF-α monoclonal antibodies and inhibitors have been shown to be effective in the treatment of psoriasis in human patients (Krueger et al (2007) N Engl J Med 356:580-592; Koutrube et al (2010) Therapeutics and Clinical Risk Management 6:123-141; Mercuri and Naldi (2010) Biologics: Targets and Therapy 4:119-129).

Multiple genome-wide association studies also have indicated NFκB activation plays a major role in psoriasis (Stuart et al (2010) Nat Gen 42, 1000-1004; Nair et al. (2009) Nat. Genet. 41(2): 199-204). In certain instances, impaired negative regulation of NFκB is due to loss of function of the inhibitory IKK (Perera et al (2012) Annu Rev Pathol Mech Dis). Many studies have shown that the NFκB signaling pathway is involved in the immune and inflammatory responses associated with psoriasis (Chen et al. (2000) J. Invest. Dermatol. 115, 1124-1133; Danning et al. (2000) Arthritis Rheum., 43, 1244-1256; 3) Aronica et al. (1999) J. Immunol., 163, 5116-5124; 4) Hawiger et al. (2001) Immunol. Res., 23, 99-109). In addition, it has been shown that several antipsoriatic drugs such as acitretin and dimethylfumart (DMF) exert their action through inhibition of the NFκB signaling pathway (Zhang et al (2008) Arch Dermatol Res. 300(10):575-81; Mrowietz et al (2005) Trend Mol Med 11(1):43-48. For example, acitretin and DMF inhibit NFκB translocation and decrease the concentration of NFκB in the nucleus of human keritinocytes. Rotterin, another potent NFκB inhibitor also possess antipsoriatic properties (Maioli et al (2010) Curr. Drug Metab. 11(5):425-30).

In some embodiments, a chemerin C15 peptide topical formulation is administered to treat psoriasis by inhibition of the production or secretion one or more cytokines involved in the pathogenesis of psoriasis. In some embodiments, a chemerin C15 peptide topical formulation is administered to treat psoriasis by inhibition of NFκB-mediated gene transcription of one or more cytokines involved in the pathogenesis of psoriasis. In some embodiments, a chemerin C15 peptide topical formulation is administered to treat inflammation associated with psoriasis.

### Dermatitis

Disclosed herein are methods of treating dermatitis in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

As used herein, dermatitis means an inflammatory condition of the skin. In certain instances, dermatitis is acute and results (either partially or fully) from contact with an offending agent. In certain instances, dermatitis is chronic and results (either partially or fully) from hypersensitivity. In some embodiments, the dermatitis is atopic dermatitis. In some embodiments, the dermatitis is contact dermatitis. In one embodiment, the dermatitis is chronic. In one embodiment, the dermatitis is acute.

In certain instances, the symptoms of dermatitis (e.g., chronic or acute) result from (either partially or fully) a disorder of an immune system. The NFκB pathway has been shown to play a critical role in the disease severity of allergic disorders (Tanaka et al (2007) J Invest Dermatol 127(4):855-63). Topical treatment of an animal models of atopic dermatitis with an NFκB inhibitor reduced hyperplasia of keratinocytes and infiltration of inflammatory cells at the site of the lesion. In addition, NFκB inhibition suppressed proliferation of immunocompetent cells, IgE production form splenic B cells and IgE activation of mast cells in vitro. In addition, downregulation of NFκB pathway by inhibitors such as licochalcone E have been shown to reduce IL-12p40 expression resulting in suppression of chronic allergic contact dermatitis.

In some embodiments, a chemerin C15 peptide topical formulation is administered to treat dermatitis by inhibition of antigen presenting cells, such as dendritic cells or macrophages. In some embodiments, a chemerin C15 peptide topical formulation is administered to treat dermatitis by inhibition of the production of one or more inflammatory cytokines. In some embodiments, a chemerin C15 peptide topical formulation is administered to treat dermatitis by inhibition NFκB-mediated gene transcription of one or more cytokines involved in the pathogenesis of dermatitis. In some embodiments, a chemerin C15 peptide topical formulation is administered to treat inflammation associated with dermatitis.

### Bullous Disorders

Disclosed herein are methods of treating bullous disorders in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

In certain instances, a bullous disorder is characterized by the formation of blisters (i.e., the accumulation of fluid between cells in the upper layers of the skin). In certain instances, bullous disorders are immune disorders in which the immune system attacks the skin and causes blistering. In certain instances, a bullous disorder is associated with the induction of an inflammatory response. High levels of cytokines such as IL-6 and TNF-α have been found in blister of patients with bullous pemphigoid (Rhodes et al. (1999) Acta Dermato-Venereologica 79(4):288).

Bullous disorders include, but are not limited to, bullous pemphigoid, pemphigus vulgaris, pemphigus vegetans, pemphigus foliaceous, paraneoplastic pemphigus, mucous membrane pemphigoid, linear IgA bullous disease, dermatitis herpeti-formis, and epidermolysis bullosa acquisita.

In some embodiments, a chemerin C15 peptide topical formulation is administered to treat inflammation associated with a bullous disorder. In some embodiments, a chemerin C15 peptide topical formulation is administered to treat a bullous disorder by inhibition of antigen presenting cells, such as dendritic cells or macrophages. In some embodiments, a chemerin C15 peptide topical formulation is administered to treat a bullous disorder through inhibition of the production of one or more inflammatory cytokines. In some embodiments, a chemerin C15 peptide topical formulation is administered to treat dermatitis through inhibition NFκB-mediated gene transcription of one or more cytokines involved in the pathogenesis of a bullous disorder.

### Eczema

Disclosed herein are methods of treating eczema in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

As used herein, eczema is a chronic inflammatory state of the skin. In some embodiments, a chemerin C15 peptide topical formulation is administered to treat inflammation associated with eczema. In some embodiments, a chemerin C15 peptide topical formulation is administered to treat eczema by inhibition of antigen presenting cells, such as dendritic cells or macrophages. In some embodiments, a chemerin C15 peptide topical formulation is administered to treat eczema through inhibition of the production of one or more inflammatory cytokines. In some embodiments, a chemerin C15 peptide topical formulation is administered to treat eczema through inhibition NFκB-mediated gene transcription of one or more cytokines involved in the pathogenesis of eczema.

### Urticaria

Disclosed herein are methods of treating urticaria in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

In certain instances, urticaria results from (either partially or fully) hypersensitivity or another immune disorder. Dermatographic urticaria is one of the most common types of urticaria in which the skin becomes raised and inflamed when scratched or rubbed.

In some embodiments, a chemerin C15 peptide topical formulation is administered to treat inflammation associated with urticaria. In some embodiments, a chemerin C15 peptide topical formulation is administered to treat urticaria by inhibition of antigen presenting cells, such as dendritic cells or macrophages. In some embodiments, a chemerin C15 peptide topical formulation is administered to treat urticaria through inhibition of the production of one or more inflammatory cytokines. In some embodiments, a chemerin C15 peptide topical formulation is administered to treat urticaria through inhibition NFκB-mediated gene transcription of one or more cytokines involved in the pathogenesis of inflammation associated with urticaria.

### Rosacea

Disclosed herein are methods of treating rosacea in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

As used herein, rosacea refers to any of erythematotelangiectatic rosacea (ETR), Papulopustular rosacea, and/or Phymatous rosacea. In some instances, rosacea is characterized by the release of release of cathelicidin antimicrobial peptides resulting in induction of proinflammatory cytokine release and an exacerbated innate immune response (Yamasaki et al. Nature Medicine 13, 975-980 (2007)).

In some embodiments, a chemerin C15 peptide topical formulation is administered to treat inflammation associated with rosacea. In some embodiments, a chemerin C15 peptide topical formulation is administered to treat rosacea by inhibition of antigen presenting cells, such as dendritic cells or macrophages. In some embodiments, a chemerin C15 peptide topical formulation is administered to treat rosacea through inhibition of the production of one or more inflammatory cytokines. In some embodiments, a chemerin C15 peptide topical formulation is administered to treat rosacea through inhibition NFκB-mediated gene transcription of one or more cytokines involved in the pathogenesis of rosacea.

### Skin Ulcers

Disclosed herein are methods of treating skin ulcers in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

As used herein, an ulcer is a disorder of the skin characterized by degradation of the epidermis and often portions of the dermis and even subcutaneous fat. In certain instances, ulcers are areas of necrotic tissue. In certain instances, ulcers result from immune system dysfunction (e.g., the improper functioning of neutrophils) and are associated with inflammation.

In some embodiments, a chemerin C15 peptide topical formulation is administered to treat inflammation associated with a skin ulcer. In some embodiments, a chemerin C15 peptide topical formulation is administered to treat a skin ulcer by inhibition of antigen presenting cells, such as dendritic cells or macrophages. In some embodiments, a chemerin C15 peptide topical formulation is administered to treat a skin ulcer through inhibition of the production of one or more inflammatory cytokines. In some embodiments, a chemerin C15 peptide topical formulation is administered to treat a skin ulcer through inhibition NFκB-mediated gene transcription of one or more cytokines involved in the pathogenesis of a skin ulcer.

### Scarring

Disclosed herein are methods of treating scarring in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

As used herein, scarring refers to the formation of a scar. In one aspect, the scar is a hypertrophic scar, or keloid scar, or a scar resulting from acne. In certain instances, a scar is an area of fibrous tissue that results from the overproduction of collagen. In certain instances, wound healing comprises the migration of fibroblasts to the site of injury. In certain instances, fibroblasts deposit collagen. In certain instances, fibroblasts deposit excess collagen at the wound site, resulting (either partially or fully) in a scar.

### Topical Formulations

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

In some embodiments, a topical formulation disclosed herein facilitates the delivery of a chemerin C15 peptide to the skin. In some embodiments, a topical formulation disclosed herein facilitates the delivery of a chemerin C15 peptide to the skin for a local effect (i.e., an effect that is limited to the skin). In certain instances, local administration of a chemerin C15 peptide reduces or eliminates side-effects that are associated with systemic administration of a chemerin C15 peptide. In some embodiments, a topical formulation of a chemerin C15 peptide disclosed herein does not result in a systemic effect, or substantially reduces the any systemic effect.

Topical formulations include, but are not limited to, aerosols, liquids, ointments, creams, lotions, solutions, suspensions, emulsions, pastes, gels, powders, salves, plasters, paints, foams, sticks, slow release nanoparticles, slow release microparticles, bioadhesives, patches, bandages and wound dressings. In some embodiments, the formulations comprise liposomes, micelles, and/or microspheres. In some embodiments, a pharmaceutically acceptable formulation includes any carrier suitable for use on human skin or mucosal surface.

### Ointments

Disclosed herein are topical ointments comprising a chemerin C15 peptide and optionally a pharmaceutically acceptable excipient. Additionally disclosed herein are methods of treating inflammatory dermatological disorders in an individual in need thereof comprising administering a topical ointment comprising a chemerin C15 peptide disclosed herein. Further disclosed herein are methods of inhibiting the activity of an inflammatory cytokine or chemokine in an individual in need thereof comprising administering a topical ointment comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical ointment comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Ointments, as is well known in the art of pharmaceutical formulation, are semi-solid preparations that are typically based on petrolatum or other petroleum derivatives. As an ointment, the composition has a consistency suitable for uniform dermal application. In some embodiments, the ointment is substantially viscous to remain in contact with the skin regardless of perspiration, excess moisture or environmental conditions. The specific ointment base to be used, as will be appreciated by those skilled in the art, is one that will provide for optimum drug delivery, and, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases are, for example, grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Some water-soluble ointment bases are prepared from polyethylene glycols of varying molecular weight; again, see Remington: The Science and Practice of Pharmacy for further information. In certain instances, ointments are semisolid preparations that soften or melt at body temperature. In certain instances, ointments re-hydrate the skin and are thus useful for dermatological disorders characterized by loss of moisture.

In some embodiments, the ointment comprises about 0.1-100 mg of chemerin C15 peptide per gram of ointment. In some embodiments, the ointment comprises about 1-10 mg of a chemerin C15 peptide per gram of ointment. In some embodiments, the ointment comprises about 1-100 mg of a chemerin C15 peptide per gram of ointment. In some embodiments, the ointment comprises about 1-10 mg of a chemerin C15 peptide per gram of ointment. In some embodiments, the chemerin C15 peptide is a human chemerin C15 peptide.

In some embodiments, the ointment comprises petrolatum. In some embodiments, the ointment comprises about 50% petrolatum. In some embodiments, the ointment comprises caprylic capric triglyceride. In some embodiments, the ointment comprises about 45% caprylic capric triglyceride. In some embodiments, the ointment comprises beeswax. In some embodiments, the ointment comprises about 5% beeswax. In some embodiments, the chemerin C15 peptide is a human chemerin C15 peptide.

In some embodiments, the ointment comprises a chemerin C15 peptide and petrolatum. In some embodiments, the ointment comprises a chemerin C15 peptide and caprylic capric triglyceride. In some embodiments, the ointment comprises a chemerin C15 peptide and beeswax. In some embodiments, the ointment comprises a chemerin C15 peptide, petrolatum, caprylic capric triglyceride, and beeswax. In one example of an ointment, the ointment comprises about 1-10 mg of a chemerin C15 peptide per gram of ointment, about 50% petrolatum, about 45% caprylic triglyceride and about 5% beeswax. In some embodiments, the chemerin C15 peptide is a human chemerin C15 peptide.

In some embodiments, the ointment comprises butylated hydroxytoluene. In some embodiments, the ointment comprises about 0.02% w/w butylated hydroxytoluene. In some embodiments, the ointment comprises PEG. In some embodiments, the ointment comprises PEG 400. In some embodiments, the ointment comprises about 15% w/w PEG 400. In some embodiments, the ointment comprises Span 80. In some embodiments, the ointment comprises about 2% w/w Span 80. In some embodiments, the ointment comprises white wax. In some embodiments, the ointment comprises about 10% white wax. In some embodiments, the ointment comprises white petrolatum. In some embodiments, the ointment comprises about 71.98% w/w white petrolatum.

In some embodiments, the ointment comprises a chemerin C15 peptide, white wax, and white petrolatum. In some embodiments, the ointment comprises a chemerin C15 peptide, butylated hydroxytoluene, PEG 400, Span 80, white wax, and white petrolatum. In an example of an ointment, the ointment comprises about 1-10 mg a chemerin C15 peptide per gram of ointment, about 0.02% w/w butylated hydroxytoluene, about 15% w/w PEG 400, about 2% w/w Span 80, about 10% w/w white wax, and about 71.98% w/w white petrolatum. In some embodiments, the chemerin C15 peptide is a human chemerin C15 peptide.

In some embodiments, the ointment comprises dimethyl isosorbide. In some embodiments, the ointment comprises about 10% w/w dimethyl isosorbide. In some embodiments, the ointment comprises butylated hydroxytoluene. In some embodiments, the ointment comprises about 0.02% w/w butylated hydroxytoluene. In some embodiments, the ointment comprises Span 80. In some embodiments, the ointment comprises about 2% w/w. In some embodiments, the ointment comprises white wax. In some embodiments, the ointment comprises about 10% w/w white wax. In some embodiments, the ointment comprises white petrolatum. In some embodiments, the ointment comprises about 76.98% w/w white petrolatum.

In some embodiments, the ointment comprises a chemerin C15 peptide, butylated dimethyl isosorbide, butylated hydroxytoluene, Span 80, white wax, and white petrolatum. In an example of an ointment, the ointment comprises about 1-10 mg of a chemerin C15 peptide per mg ointment, about 10% w/w dimethyl isosorbide, about 0.02% w/w butylated hydroxytoluene, about 2% w/w Span 80, about 10% w/w white wax, and about 76.98% w/w white petrolatum. In some embodiments, the chemerin C15 peptide is a human chemerin C15 peptide.

### Solutions

Disclosed herein are topical solutions comprising a chemerin C15 peptide and optionally a pharmaceutically acceptable excipient. Additionally disclosed herein are methods of treating inflammatory dermatological disorders in an individual in need thereof comprising administering a topical solution comprising a chemerin C15 peptide disclosed herein. Further disclosed herein are methods of inhibiting the activity of an inflammatory cytokine or chemokine in an individual in need thereof comprising administering a topical solution comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical solution comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Solutions, as well known in the art, are homogenous liquids comprising dissolved materials. In certain embodiments, solutions are water or organic solvent based. In certain embodiments, solutions comprise a chemerin C15 peptide along with additional components which enhance the penetration of a chemerin C15 peptide applied topically to the skin. In some embodiments, a solution comprising a chemerin C15 peptide is applied topically to the skin by painting with an applicator, as drops or as a spray. In some embodiments, the solution is applied from a pump spray bottle. In some embodiments, the solution is applied from an eye dropper.

In some embodiments, the solution comprises about 0.1-100 mg of a chemerin C15 peptide per mL of solution. In some embodiments, the solution comprises about 1-10 mg of a chemerin C15 peptide per mL of solution. In some embodiments, the solution comprises about 1-100 mg of a chemerin C15 peptide per mL of solution. In some embodiments, the solution comprises about 1-10 mg of a chemerin C15 peptide per mL of solution. In some embodiments, the chemerin C15 peptide is a human chemerin C15 peptide.

In some embodiments, the solution comprises isopropyl myristate. In some embodiments, the solution comprises alcohol. In some embodiments, the solution comprises undecylenic acid. In some embodiments, the solution comprises sodium lauryl sulfate.

In some embodiments, the solution comprises a chemerin C15 peptide, isopropyl myristate, alcohol, undecylenic acid and sodium lauryl sulfate. In one example of a solution, the solution contains about 1-10 mg of a chemerin C15 peptide per mL of solution, isopropyl myristate, alcohol, undecylenic acid and sodium lauryl sulfate. In some embodiments, the chemerin C15 peptide is a human chemerin C15 peptide.

In some embodiments, the solution comprises isopropyl myristate. In some embodiments, the solution comprises about 45% isopropyl myristate. In some embodiments, the solution comprises isopropyl myristate alcohol. In some embodiments, the solution comprises about 45% isopropyl myristate alcohol. In some embodiments, the solution comprises undecylenic acid. In some embodiments, the solution comprises about 5% undecylenic acid. In some embodiments, the solution comprises sodium lauryl sulfate. In some embodiments, the solution comprises about 5% sodium lauryl sulfate.

In some embodiments, the solution comprises a chemerin C15 peptide, isopropyl myristate, alcohol, undecylenic acid, and sodium lauryl sulfate. In another example of a solution, the solution comprises about 1-10 mg of a chemerin C15 peptide per mL of solution, about 45% isopropyl myristate, about 45% alcohol, about 5% undecylenic acid and about 5% sodium lauryl sulfate. In some embodiments, the chemerin C15 peptide is a human chemerin C15 peptide. In some embodiments, the solution is applied from a pump spray bottle.

In some embodiments, the solution comprises a chemerin C15 peptide, DMSO and water. In a another example of a solution, the solution comprises about 1-10 mg of a chemerin C15 peptide per mL of solution, about 50% DMSO, and about 50% water. In some embodiments, the chemerin C15 peptide is a human chemerin C15 peptide. In some embodiments, the solution is applied from a pump spray bottle.

In another example of a solution, the solution comprises about 1-10 mg of a chemerin C15 peptide per ml solution in DMSO. In some embodiments, the chemerin C15 peptide is a human chemerin C15 peptide. In some embodiments, the solution is applied from a pump spray bottle.

In some embodiments, the solution comprises dimethyl isosorbide. In some embodiments, the solution comprises about 15% w/w dimethyl isosorbide. In some embodiments, the solution comprises Transcutol. In some embodiments, the solution comprises about 25% w/w Transcutol. In some embodiments, the solution comprises hexylene glycol. In some embodiments, the solution comprises about 12% w/w hexylene glycol. In some embodiments, the solution comprises propylene glycol. In some embodiments, the solution comprises about 5% w/w propylene glycol.

In some embodiments, the solution comprises dimethyl isosorbide, Transcutol, hexylene glycol, and propylene glycol. In another example of a solution, the solution comprises about 1-10 mg chemerin C15 peptide per ml solution, about 15% w/w dimethyl isosorbide, about 25% w/w Transcutol, about 12% w/w hexylene glycol, about 5% w/w propylene glycol, 25% Trolamine q.s. pH 4.5 and water to 100%. In some embodiments, the chemerin C15 peptide is a human chemerin C15 peptide. In some embodiments, the solution is applied from a pump spray bottle.

In another example of a solution, the solution comprises about 1-10 mg chemerin C15 peptide per ml solution, about 15% w/w Dimethyl isosorbide, about 25% w/w Transcutol, about 12% w/w Hexylene glycol, about 5% w/w Propylene glycol, 25% Trolamine q.s. pH 6.0 and water to 100%. In some embodiments, the chemerin C15 peptide is a human chemerin C15 peptide. In some embodiments, the solution is applied from a pump spray bottle.

### Creams and Lotions

Disclosed herein are topical creams or lotions comprising a chemerin C15 peptide and optionally a pharmaceutically acceptable excipient. Additionally disclosed herein are methods of treating inflammatory dermatological disorders in an individual in need thereof comprising administering a topical creams or lotions comprising a chemerin C15 peptide disclosed herein. Further disclosed herein are methods of inhibiting the activity of an inflammatory cytokine or chemokine in an individual in need thereof comprising administering a topical creams or lotions comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical creams or lotions comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Creams, as also well known in the art, are viscous liquids or semi-solid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant. In certain instances, creams are semisolid (e.g., soft solid or thick liquid) formulations that include a chemerin C15 peptide dispersed in an oil-in-water emulsion or a water-in-oil emulsion. Disclosed herein, in certain embodiments, is a topical formulation of a chemerin C15 peptide wherein the topical formulation is in the form of a lotion. In certain instances, lotions are fluid emulsions (e.g., oil-in-water emulsions or a water-in-oil emulsions). In some embodiments, the hydrophobic component of a lotion and/or cream is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly).

In certain instances, lotions and creams have a “drying” effect on dermatological disorders (e.g., some or all fluid exuded from the disorder is miscible in the ointment) and are thus useful for dermatological disorders characterized by the exudation of fluids.

In some embodiments, the cream comprises about 0.1-100 mg of a chemerin C15 peptide per ml cream. In some embodiments, the cream comprises about 1-10 mg of a chemerin C15 peptide per ml cream. In some embodiments, the cream comprises about 1-100 mg of a chemerin C15 peptide per ml cream. In some embodiments, the cream comprises about 1-10 mg of a chemerin C15 peptide per ml cream. In some embodiments, the chemerin C15 peptide is a human chemerin C15 peptide.

In some embodiments, the lotion comprises about 0.1-100 mg of a chemerin C15 peptide per ml lotion. In some embodiments, the lotion comprises about 1-10 mg of a chemerin C15 peptide per ml lotion. In some embodiments, the lotion comprises about 1-100 mg of a chemerin C15 peptide per ml lotion. In some embodiments, the lotion comprises about 1-10 mg of a chemerin C15 peptide per ml lotion. In some embodiments, the chemerin C15 peptide is a human chemerin C15 peptide.

In some embodiments, the lotion comprises dimethyl isosorbide. In some embodiments, the lotion comprises about 13% w/w dimethyl isosorbide. In some embodiments, the lotion comprises Transcutol. In some embodiments, the lotion comprises about 20% w/w Transcutol. In some embodiments, the lotion comprises Hexylene glycol. In some embodiments, the lotion comprises about 10% w/w Hexylene glycol. In some embodiments, the lotion comprises Propylene glycol. In some embodiments, the lotion comprises about 4% w/w Propylene glycol. In some embodiments, the lotion comprises Methylparaben. In some embodiments, the lotion comprises about 0.015% w/w Methylparaben. In some embodiments, the lotion comprises Propylparaben. In some embodiments, the lotion comprises about 0.05% w/w Propylparaben. In some embodiments, the lotion comprises EDTA. In some embodiments, the lotion comprises about 0.01% w/w EDTA. In some embodiments, the lotion comprises Carbopol Ultrez 10. In some embodiments, the lotion comprises about 0.5% w/w Carbopol Ultrez 10. In some embodiments, the lotion comprises Penmulen TR-1. In some embodiments, the lotion comprises about 0.2% w/w Penmulen TR-1. In some embodiments, the lotion comprises Isopropyl myristate. In some embodiments, the lotion comprises about 3% w/w Isopropyl myristate. In some embodiments, the lotion comprises Oleyl alcohol. In some embodiments, the lotion comprises about 5% w/w Oleyl alcohol. In some embodiments, the lotion comprises about 0.2% w/w Butylated hydroxytoluene. In some embodiments, the lotion comprises White petrolatum. In some embodiments, the lotion comprises about 5% w/w White petrolatum. In some embodiments, the pH of the lotion is adjusted to about 4.0 to 6.0. with Trolamine. In some embodiments, the pH of the lotion is adjusted to about 4.0 to 6.0 with Trolamine.

In some embodiments, the lotion comprises a chemerin C15 peptide, Dimethyl isosorbide, Transcutol, Hexylene glycol, Propylene glycol, Methylparaben, Propylparaben, EDTA, Carbopol Ultrez 10, Penmulen TR-1, and Butylated hydroxytoluene. In some embodiments, the lotion comprises a chemerin C15 peptide, Dimethyl isosorbide, Transcutol, Hexylene glycol, Propylene glycol, Methylparaben, Propylparaben, EDTA, Carbopol Ultrez 10, Penmulen TR-1, Isopropyl myristate, Oleyl alcohol, Butylated hydroxytoluene, and White petrolatum. In some embodiments, the chemerin C15 peptide is a human chemerin C15 peptide.

In one example of a lotion, the lotion comprises about 1-10 mg of a chemerin C15 peptide per ml lotion, about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.5% w/w Carbopol Ultrez 10, about 0.2% w/w Penmulen TR-1, about 3% w/w Isopropyl myristate, about 5% w/w Oleyl alcohol, about 0.2% w/w Butylated hydroxytoluene, about 5% w/w White petrolatum, 25% Trolamine q.s. pH 6.0 and water to 100%. In some embodiments, the chemerin C15 peptide is a human chemerin C15 peptide.

In some embodiments, the lotion comprises Cetyl alcohol. In some embodiments, the lotion comprises about 2% w/w Cetyl alcohol. In some embodiments, the lotion comprises Light mineral oil. In some embodiments, the lotion comprises about 5.5% w/w Light mineral oil. In some embodiments, the lotion comprises Oleic acid. In some embodiments, the lotion comprises about 5% w/w Oleic acid.

In some embodiments, the lotion comprises a chemerin C15 peptide, Dimethyl isosorbide, Transcutol, Hexylene glycol, Propylene glycol, Methylparaben, Propylparaben, EDTA, Carbopol Ultrez 10, Penmulen TR-1, Cetyl alcohol, Light mineral oil, Oleic acid, Butylated hydroxytoluene. In some embodiments, the chemerin C15 peptide is a human chemerin C15 peptide.

In another example of a lotion, the lotion comprises about 1-10 mg of a chemerin C15 peptide per ml lotion, about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.3% w/w Carbopol Ultrez 10, about 0.2% w/w Penmulen TR-1, about 2% w/w Cetyl alcohol, about 5.5% w/w Light mineral oil, about 5% w/w Oleic acid, 0.2% w/w Butylated hydroxytoluene, 25% Trolamine q.s. pH 6.0 and water to 100%. In some embodiments, the chemerin C15 peptide is a human chemerin C15 peptide. In some embodiments, the chemerin C15 peptide is a human chemerin C15 peptide.

### Gels

Disclosed herein are topical gels comprising a chemerin C15 peptide and optionally a pharmaceutically acceptable excipient. Additionally disclosed herein are methods of treating inflammatory dermatological disorders in an individual in need thereof comprising administering a topical gel comprising a chemerin C15 peptide disclosed herein. Further disclosed herein are methods of inhibiting the activity of an inflammatory cytokine or chemokine in an individual in need thereof comprising administering a topical gel comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical gel comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Gels are semi-solid, suspension-type systems and are well known in the art. Gel forming agent for use herein can be any gelling agent typically used in the pharmaceutical art for topical semi solid dosage forms. Single-phase gels contain organic macromolecules distributed substantially uniformly throughout the carrier liquid, which is typically aqueous, but also can contain an alcohol and optionally an oil. In order to prepare a uniform gel, dispersing agents such as alcohol or glycerin can be added, or the gelling agent can be dispersed by tritration, mechanical mixing or stirring, or combinations thereof. The amount of gelling agents varies widely and will ordinarily range from about 0.1% to about 2.0% by weight, based on the total weight of the composition. The gel forming agent also works by the principle of copolymerization. Under alkaline pH, carbomer in presence of water undergoes cross linking and forms a gel like structure. The degree of polymerization is dependent upon the pH. At a threshold pH, the viscosities achieved by the polymer grade are the maximum. In certain instances, gels are semisolid (or semi-rigid) systems consisting of dispersions of large organic molecules dispersed in a liquid. In certain instances, gels are water-soluble and are removed using warm water or saline. In certain instances, gels re-hydrate the skin and are thus useful for dermatological disorders characterized by loss of moisture.

In some embodiments, the gel comprises about 0.1-100 mg of a chemerin C15 peptide per ml gel. In some embodiments, the gel comprises about 1-10 mg of a chemerin C15 peptide per ml gel. In some embodiments, the gel comprises about 1-100 mg of a chemerin C15 peptide per ml gel. In some embodiments, the gel comprises about 1-10 mg of a chemerin C15 peptide per ml gel. In some embodiments, the chemerin C15 peptide is a human chemerin C15 peptide.

In some embodiments, the lotion comprises dimethyl isosorbide. In some embodiments, the lotion comprises about 15% w/w dimethyl isosorbide. In some embodiments, the lotion comprises Transcutol. In some embodiments, the lotion comprises about 25% w/w Transcutol. In some embodiments, the lotion comprises Hexylene glycol. In some embodiments, the lotion comprises about 12% w/w Hexylene glycol. In some embodiments, the lotion comprises Propylene glycol. In some embodiments, the lotion comprises about 5% w/w Propylene glycol. In some embodiments, the lotion comprises Methylparaben. In some embodiments, the lotion comprises about 0.015% w/w Methylparaben. In some embodiments, the lotion comprises Propylparaben. In some embodiments, the lotion comprises about 0.05% w/w Propylparaben. In some embodiments, the gel comprises EDTA. In some embodiments, the gel comprises about 0.01% w/w EDTA. In some embodiments, the gel comprises Penmulen TR-1. In some embodiments, the gel comprises about 0.5% w/w Penmulen TR-1. In some embodiments, the gel comprises Hydroxyethyl cellulose. In some embodiments, the gel comprises about 1% w/w Hydroxyethyl cellulose.

In some embodiments, the gel comprises a chemerin C15 peptide, Dimethyl isosorbide, Transcutol, Hexylene glycol, Propylene glycol, Methylparaben, Propylparaben, and EDTA. In some embodiments, the gel comprises a chemerin C15 peptide, Dimethyl isosorbide, Transcutol, Hexylene glycol, Propylene glycol, Methylparaben, Propylparaben, EDTA, and Penmulen TR-1. In some embodiments, the chemerin C15 peptide is a human chemerin C15 peptide.

In one example of a gel, the gel comprises about 1-10 mg of a chemerin C15 peptide per ml gel, about 15% w/w Dimethyl isosorbide, about 25% w/w Transcutol, about 12% w/w Hexylene glycol, about 5% w/w Propylene glycol, about 0.015% w/w Methylparaben, about 0.05% w/w Propylparaben, about 0.01% w/w EDTA, about 0.5% w/w Penmulen TR-1, 25% Trolamine q.s. pH 6.0 and water to 100%. In some embodiments, the chemerin C15 peptide is a human chemerin C15 peptide.

In some embodiments, the gel comprises a chemerin C15 peptide, Dimethyl isosorbide, Transcutol, Hexylene glycol, Propylene glycol, Methylparaben, Propylparaben, EDTA, and hydroxyethylcellulose. In some embodiments, the chemerin C15 peptide is a human chemerin C15 peptide.

In another example of a gel, the gel comprises about 1-10 mg of a chemerin C15 peptide per ml gel, about 15% w/w Dimethyl isosorbide, about 25% w/w Transcutol, about 12% w/w Hexylene glycol, about 5% w/w Propylene glycol, about 0.015% w/w Methylparaben, about 0.05% w/w Propylparaben, about 0.01% w/w EDTA, about 1% w/w Hydroxyethyl cellulose, 25% Trolamine q.s. pH 4.5 and water to 100%. In some embodiments, the chemerin C15 peptide is a human chemerin C15 peptide.

### Pastes

Disclosed herein are topical pastes comprising a chemerin C15 peptide and optionally a pharmaceutically acceptable excipient. Additionally disclosed herein are methods of treating inflammatory dermatological disorders in an individual in need thereof comprising administering a topical paste comprising a chemerin C15 peptide disclosed herein. Further disclosed herein are methods of inhibiting the activity of an inflammatory cytokine or chemokine in an individual in need thereof comprising administering a topical paste comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical paste comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Pastes are semi-solid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base. In certain instances, pastes contain at least 20% solids. In certain instances, pastes are ointments that do not flow at body temperature. In certain instances, pastes re-hydrate the skin and are thus useful for dermatological disorders characterized by loss of moisture. In certain instances, pastes serve as protective coatings over areas to which they are applied.

In some embodiments, the solution comprises about 0.1-100 mg of a chemerin C15 peptide per gram paste. In some embodiments, the solution comprises about 1-10 mg of a chemerin C15 peptide per gram paste. In some embodiments, the solution comprises about 1-100 mg of a chemerin C15 peptide per gram paste. In some embodiments, the solution comprises about 1-10 mg of a chemerin C15 peptide per gram paste. In some embodiments, the chemerin C15 peptide is a human chemerin C15 peptide.

### Plasters

Disclosed herein are topical plasters comprising a chemerin C15 peptide and optionally a pharmaceutically acceptable excipient. Additionally disclosed herein are methods of treating inflammatory dermatological disorders in an individual in need thereof comprising administering a topical plaster comprising a chemerin C15 peptide disclosed herein. Further disclosed herein are methods of inhibiting the activity of an inflammatory cytokine or chemokine in an individual in need thereof comprising administering a topical plaster comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical plaster comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Plasters are comprised of a pasty mixture that is spread on the body, either directly or after being saturated into a base material such as cloth. In some embodiments, medications, including the pharmacologically active compositions of the invention, are dissolved or dispersed within the plaster to make a medicated plaster.

In some embodiments, the plaster comprises about 0.1-100 mg of a chemerin C15 peptide per gram plaster. In some embodiments, the plaster comprises about 1-10 mg of a chemerin C15 peptide per gram plaster. In some embodiments, the plaster comprises about 1-100 mg of a chemerin C15 peptide per gram plaster. In some embodiments, the plaster comprises about 1-10 mg of a chemerin C15 peptide per gram plaster. In some embodiments, the chemerin C15 peptide is a human chemerin C15 peptide.

### Sticks

Disclosed herein are topical sticks comprising a chemerin C15 peptide and optionally a pharmaceutically acceptable excipient. Additionally disclosed herein are methods of treating inflammatory dermatological disorders in an individual in need thereof comprising administering a topical stick comprising a chemerin C15 peptide disclosed herein. Further disclosed herein are methods of inhibiting the activity of an inflammatory cytokine or chemokine in an individual in need thereof comprising administering a topical stick comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical stick comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

In certain instances, sticks are solid dosage forms that melt at body temperature. In some embodiments, a stick comprises a wax, a polymer, a resin, dry solids fused into a firm mass, and/or fused crystals. In some embodiments, a topical formulation of a chemerin C15 peptide is in the form of a styptic pencil (i.e., a stick prepared by (1) heating crystals until they lose their water of crystallization and become molten, and (2) pouring the molten crystals into molds and allowing them to harden). In some embodiments, a topical formulation of a chemerin C15 peptide is in the form of stick wherein the stick comprises a wax (e.g., the wax is melted and poured into appropriate molds in which they solidify in stick form).

In some embodiments, a topical formulation of a chemerin C15 peptide is in the form of stick wherein the stick comprises a melting base (i.e., a base that softens at body temperature). Examples of melting bases include, but are not limited to, waxes, oils, polymers and gels. In some embodiments, a topical formulation of a chemerin C15 peptide is in the form of stick wherein the stick comprises a moisten base (i.e., a base that is activated by the addition of moisture).

In some embodiments, the solution comprises about 0.1-100 mg of a chemerin C15 peptide per gram of the stick. In some embodiments, the solution comprises about 1-10 mg of a chemerin C15 peptide per gram of the stick. In some embodiments, the solution comprises about 1-100 mg of a chemerin C15 peptide per gram of the stick. In some embodiments, the solution comprises about 1-10 mg of a chemerin C15 peptide per gram of the stick. In some embodiments, the chemerin C15 peptide is a human chemerin C15 peptide.

### Bioadhesives

Disclosed herein are topical bioadhesives comprising a chemerin C15 peptide and optionally a pharmaceutically acceptable excipient. Additionally disclosed herein are methods of treating inflammatory dermatological disorders in an individual in need thereof comprising administering a topical bioadhesive comprising a chemerin C15 peptide disclosed herein. Further disclosed herein are methods of inhibiting the activity of an inflammatory cytokine or chemokine in an individual in need thereof comprising administering a topical bioadhesive comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a topical bioadhesive comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Bioadhesives are preparations that adhere to surfaces of body tissues. Polymeric bioadhesive formulations are well known in the art; see, for example, Heller et al., “Biodegradable polymers as drug delivery systems”, in Chasin, M. and Langer, R., eds.: Dekker, N.Y., pp. 121-161 (1990); and U.S. Pat. No. 6,201,065. Suitable non-polymeric bioadhesives are also known in the art, including certain fatty acid esters (U.S. Pat. No. 6,228,383).

Disclosed herein, in certain embodiments, is a topical formulation of a chemerin C15 peptide wherein the topical formulation is administered via a patch. In some embodiments, a topical formulation disclosed herein is dissolved and/or dispersed in a polymer or an adhesive. In some embodiments, a patch disclosed herein is constructed for continuous, pulsatile, or on demand delivery of a chemerin C15 peptide.

In some embodiments, the bioadhesive comprises about 0.1-100 mg of a chemerin C15 peptide. In some embodiments, the bioadhesive comprises about 1-10 mg of a chemerin C15 peptide. In some embodiments, the bioadhesive comprises about 1-100 mg of a chemerin C15 peptide. In some embodiments, the bioadhesive comprises about 1-10 mg of a chemerin C15 peptide. In some embodiments, the chemerin C15 peptide is a human chemerin C15 peptide.

### Patches, Wound Dressings, and Bandages

Disclosed herein are patches, wound dressings or bandages comprising a chemerin C15 peptide and optionally a pharmaceutically acceptable excipient. Additionally disclosed herein are methods of treating inflammatory dermatological disorders in an individual in need thereof comprising administering a patch, would dressing or bandage comprising a chemerin C15 peptide disclosed herein. Further disclosed herein are methods of inhibiting the activity of an inflammatory cytokine or chemokine in an individual in need thereof comprising administering a patch, would dressing or bandage comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a patch, would dressing or bandage comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

Wound dressings, patches and bandages include, but are not limited to gauzes, transparent film dressings, hydrogels, polyurethane foam dressings, hydrocolloids and alginates. In certain instances, wound dressings (1) maintain moisture in the wound, (2) are semipermeable, (3) are semiocclusive, (4) allow for autolytic debridement, (5) protect from external contaminants, (6) absorb exuded fluids, and/or (7) allow for wound visualization.

In some embodiments, the patch, wound dressing, or bandage comprises about 0.1-100 mg of a chemerin C15 peptide. In some embodiments, the patch, wound dressing, or bandage comprises about 1-10 mg of a chemerin C15 peptide. In some embodiments, the patch, wound dressing, or bandage comprises about 1-100 mg of a chemerin C15 peptide. In some embodiments, the patch, wound dressing, or bandage comprises about 1-10 mg of a chemerin C15 peptide. In some embodiments, the chemerin C15 peptide is a human chemerin C15 peptide.

### Dermatological Excipients

Disclosed herein are topical formulations comprising a chemerin C15 peptide and a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein and a pharmaceutically acceptable excipient. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein and a pharmaceutically acceptable excipient. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein and a pharmaceutically acceptable excipient. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

In some embodiments, the topical formulations described herein comprise one or more inert excipients, which include, but are not limited to, water, buffered aqueous solutions, surfactants, volatile liquids, starches, polyols, granulating agents, microcrystalline cellulose, diluents, lubricants, acids, bases, salts, emulsions, such as oil/water emulsions, oils such as mineral oil and vegetable oil, wetting agents, chelating agents, antioxidants, sterile solutions, complexing agents, and disintegrating agents.

In some embodiments, the topical formulations described herein comprise one or more cosmetic or pharmaceutical agents commonly used in the skin care industry. Examples of such agents are described in, for example, CTFA Cosmetic Ingredient Handbook, Seventh Edition, 1997 and the Eighth Edition, 2000, which is incorporated by reference herein in its entirety. Examples of classes of such agents include, but are not limited to: abrasives, absorbents, aesthetic components such as fragrances, pigments, colorings/colorants, essential oils, skin sensates, astringents, etc. (e.g. clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, cosmetic biocides, denaturants, drug astringents, external analgesics, film formers or materials, opacifying agents, pH adjusters, propellants, reducing agents, sequestrants, skin bleaching and lightening agents (e.g. hydroquinone, kojic acid, ascorbic acid, magnesium ascorbyl phosphate, ascorbyl glucosamine), skin-conditioning agents (e.g. humectants), skin soothing and/or healing agents (e.g. panthenol and its derivatives, aloe vera, pantothenic acid and its derivatives, allantoin, bisabolol, and dipotassium glycyrrhizinate), skin protectants (e.g., sunscreens, or ultraviolet light absorbers or scattering agents), skin treating agents, thickeners, and vitamins and derivatives thereof. In some embodiments, a topical formulation of a chemerin C15 peptide comprises one or more of such agents.

In some embodiments, the topical formulations described herein comprise a gelling (or thickening) agent. In some embodiments, a topical formulation disclosed herein further comprises from about 0.1% to about 5%, more preferably from about 0.1% to about 3%, and most preferably from about 0.25% to about 2%, of a gelling agent. In certain embodiments, the viscosity of a topical formulation disclosed herein is in the range from about 100 to about 500,000 cP, about 100 cP to about 1,000 cP, about 500 cP to about 1500 cP, about 1000 cP to about 3000 cP, about 2000 cP to about 8,000 cP, about 4,000 cP to about 10,000 cP, about 10,000 cP to about 50,000 cP.

Suitable gelling agents for use in preparation of the gel topical formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

In some embodiments, the topical formulations described herein comprise an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kikui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

In some embodiments, the topical formulations described herein comprise an anti-oxidant. Anti-oxidants include, but are not limited to, propyl, octyl and dodecyl esters of gallic acid, butylated hydroxyanisole (BHA, usually purchased as a mixture of ortho and meta isomers), green tea extract, uric acid, cysteine, pyruvate, nordihydroguaiaretic acid, ascorbic acid, salts of ascorbic acid such as ascorbyl palmitate and sodium ascorbate, ascorbyl glucosamine, vitamin E (i.e., tocopherols such as a-tocopherol), derivatives of vitamin E (e.g., tocopheryl acetate), retinoids such as retinoic acid, retinol, trans-retinol, cis-retinol, mixtures of trans-retinol and cis-retinol, 3-dehydroretinol and derivatives of vitamin A (e.g., retinyl acetate, retinal and retinyl palmitate, also known as tetinyl palmitate), sodium citrate, sodium sulfite, lycopene, anthocyanids, bioflavinoids (e.g., hesperitin, naringen, rutin and quercetin), superoxide dismutase, glutathione peroxidase, butylated hydroxytoluene (BHT), indole-3-carbinol, pycnogenol, melatonin, sulforaphane, pregnenolone, lipoic acid and 4-hydroxy-5-methyl-3[2H]-furanone.

In some embodiments, the topical formulations described herein comprise a skin protecting agent. Exemplary skin protecting agent include, but are not limited to, sunscreens, anti-acne additives, anti-wrinkle and anti-skin atrophy agents. Suitable sunscreens as skin protecting agents include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomethyl salicylate, octyl salicylate, 4,4′-methoxy-t-butyldibenzoylmethane, 4-isopropyldibenzoylmethane, 3-benzylidene camphor, 3-(4-methylbenzylidene) camphor, anthanilates, ultrafine titanium dioxide, zinc oxide, iron oxide, silica, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone and 4-N,N(2-ethylhexyl)-methylaminobenzoic acid ester of 4-(2-hydroxyethoxy)dibenzoylmethane. Suitable anti-acne agents include salicylic acid; 5-octanoyl salicylic acid; resorcinol; retinoids such as retinoic acid and its derivatives; sulfur-containing D and L amino acids other than cysteine; lipoic acid; antibiotics and antimicrobials such as benzoyl peroxide, octopirox, tetracycline, 2,4,4′-trichloro-2′-hydroxydiphenyl ether, 3,4,4′-trichlorobanilide, azelaic acid, phenoxyethanol, phenoxypropanol, phenoxisopropanol, ethyl acetate, clindamycin and melclocycline; flavonoids; and bile salts such as scymnol sulfate, deoxycholate and cholate. Examples of anti-wrinkle and anti-skin atrophy agents are retinoic acid and its derivatives, retinol, retinyl esters, salicylic acid and its derivatives, sulfur-containing D and L amino acids except cysteine, alpha-hydroxy acids (e.g., glycolic acid and lactic acid), phytic acid, lipoic acid and lysophosphatidic acid.

In some embodiments, the topical formulations described herein comprise irritation-mitigating additives to minimize or eliminate the possibility of skin irritation or skin damage resulting from the permeation-enhancing base or other components of the composition. Exemplary irritation-mitigating additives include, but are not limited to, alpha-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine.

In some embodiments, the topical formulations described herein comprise a dry-feel modifier, which is an agent which when added to an emulsion, imparts a “dry feel” to the skin when the emulsion dries. Exemplary dry-feel modifiers include, but are not limited to, talc, kaolin, chalk, zinc oxide, silicone fluids, inorganic salts such as barium sulfate, surface treated silica, precipitated silica, fumed silica such as an Aerosil available from Degussa Inc. of New York, N.Y. U.S.A. Another dry feel modifier is an epichlorohydrin cross-linked glyceryl starch of the type that is disclosed in U.S. Pat. No. 6,488,916.

In some embodiments, the topical formulations described herein comprise an antimicrobial agent to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, purite, peroxides, perborates and combinations thereof.

In some embodiments, the topical formulations described herein comprise an aesthetic agent. Examples of aesthetic agents include fragrances, pigments, colorants, essential oils, skin sensates and astringents. Suitable aesthetic agents include clove oil, menthol, camphor, eucalyptus oil, eugenol, methyl lactate, bisabolol, witch hazel distillate and green tea extract.

In some embodiments, the topical formulations described herein comprise a fragrance. Fragrances are aromatic substances which can impart an aesthetically pleasing aroma. Typical fragrances include aromatic materials extracted from botanical sources (i.e., rose petals, gardenia blossoms, jasmine flowers, etc.) which can be used alone or in any combination to create essential oils. In some embodiment, alcoholic extracts are prepared for compounding fragrances. In some examples, the fragrance is a synthetically prepared fragrance. One or more fragrances can optionally be included in the sunscreen composition in an amount ranging from about 0.001 to about 5 weight percent, p or about 0.01 to about 0.5 percent by weight. In some embodiments, additional preservatives are used if desired and include, for example, well known preservative compositions such as benzyl alcohol, phenyl ethyl alcohol and benzoic acid, diazolydinyl, urea, chlorphenesin, iodopropynyl and butyl carbamate, among others.

In some embodiments, the topical formulations described herein comprise a surfactant. Surfactants which can be used to form pharmaceutical compositions and dosage forms provides herein include, but are not limited to, hydrophilic surfactants, lipophilic surfactants, and mixtures thereof. In some embodiments, a mixture of hydrophilic surfactants is employed. In some embodiments, a mixture of lipophilic surfactants is employed. In some embodiments, a mixture of at least one hydrophilic surfactant and at least one lipophilic surfactant is employed.

In certain embodiments, the surfactant is any suitable, non-toxic compound that is non-reactive with the medicament and that substantially reduces the surface tension between the medicament, the excipient and the site of administration. Exemplary surfactants include but are not limited to: oleic acid available under the tradenames Mednique 6322 and Emersol 6321 (from Cognis Corp., Cincinnati, Ohio); cetylpyridinium chloride (from Arrow Chemical, Inc. Westwood, N.J.); soya lecithin available under the tradename Epikuron 200 (from Lucas Meyer Decatur, Ill.); polyoxyethylene(20) sorbitan monolaurate available under the tradename Tween 20 (from ICI Specialty Chemicals, Wilmington, Del.); polyoxyethylene(20) sorbitan monostearate available under the tradename Tween 60 (from ICI); polyoxyethylene(20) sorbitan monooleate available under the tradename Tween 80 (from ICI); polyoxyethylene (10) stearyl ether available under the tradename Brij 76 (from ICI); polyoxyethylene (2) oleyl ether available under the tradename Brij 92 (frown ICI); Polyoxyethylene-polyoxypropylene-ethylenediamine block copolymer available under the tradename Tetronic 150 R1 (from BASF); polyoxypropylene-polyoxyethylene block copolymers available under the tradenames Pluronic L-92, Pluronic L-121 end Pluronic F 68 (from BASF); castor oil ethoxylate available under the tradename Alkasurf CO-40 (from Rhone-Poulenc Mississauga Ontario, Canada); and mixtures thereof.

In some embodiment a suitable hydrophilic surfactant has an HLB value of at least 10, while suitable lipophilic surfactants have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. An HLB value of a surfactant is guide generally used to enable formulation of industrial, pharmaceutical and cosmetic emulsions.

Hydrophilic surfactants for use in the topical formulations provided are either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Exemplary ionic surfactants include lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

In some embodiments, ionic surfactants are ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Exemplary hydrophilic non-ionic surfactants include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. In some embodiments, the polyol is glycerol, ethylene glycol, polyethylene glycol, sorbitol, propylene glycol, pentaerythritol, or a saccharide.

Other exemplary hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Exemplary suitable lipophilic surfactants include, but are not limited to fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some embodiments, surfactants are used in any formulation provided herein where its use is not otherwise contradicted. In some embodiments, the surfactant is in an amount of about 0.0001 to 1% by weight, in particular about 0.001 to 0.1% by weight, based on the total weight of the formulation. In some embodiments, the use of no surfactants or limited classes of surfactants is desirable. In some embodiments, the topical formulations provided can contain no, or substantially no surfactant, i.e. contain less than approximately 0.0001% by weight of surface-active agents. This is particularly the case if one employs a cromone as described above. Other suitable surfactant/emulsifying agents would be known to one of skill in the art and are listed in the CTFA International Cosmetic Ingredient Dictionary and Handbook, Vol. 2, 7th Edition (1997).

Other exemplary suitable aqueous vehicles include, but are not limited to, Ringer's solution and isotonic sodium chloride. In some embodiments, aqueous suspensions include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Exemplary chelating agents which can be used to form pharmaceutical compositions and dosage forms provide herein include, but are not limited to, ethylene diaminetetraacetic acid (EDTA), EDTA disodium, calcium disodium edetate, EDTA trisodium, albumin, transferrin, desferoxamine, desferal, desferoxamine mesylate, EDTA tetrasodium and EDTA dipotassium, sodium metasilicate or combinations of any of these. In some embodiments, up to about 0.1% W/V of a chelating agent, such as EDTA or its salts, is added to the formulations of the invention.

Exemplary preservatives which can be used to form pharmaceutical compositions and dosage forms provided herein include, but are not limited to, purite, peroxides, perborates, imidazolidinyl urea, diazolidinyl urea, phenoxyethanol, alkonium chlorides including benzalkonium chlorides, methylparaben, ethylparaben and propylparaben. In other embodiments, suitable preservatives for the compositions of the invention include: benzalkonium chloride, purite, peroxides, perborates, thimerosal, chlorobutanol, methyl paraben, propyl paraben, phenylethyl alcohol, edetate disodium, sorbic acid, Onamer M, or other agents known to those skilled in the art. In some embodiments of the invention, such preservatives are employed at a level of from 0.004% to 0.02% W/V.

Exemplary lubricants which can be used to form pharmaceutical compositions and dosage forms provided include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof.

Exemplary thickening agents which can be used to form pharmaceutical compositions and dosage forms provided include, but are not limited to, isopropyl myristate, isopropyl palmitate, isodecyl neopentanoate, squalene, mineral oil, C₁₂-C₁₅ benzoate and hydrogenated polyisobutene. In some embodiments, agents which would not disrupt other compounds of the final product, such as non-ionic thickening agents are desirable. The selection of additional thickening agents is well within the skill of one in the art.

Pharmaceutical topical formulations disclosed herein are formulated in any suitable manner. Any suitable technique, carrier, and/or excipient is contemplated for use with the chemerin C15 peptides disclosed herein. For a summary of pharmaceutical topical formulations described herein see Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), which are herein incorporated by reference for such disclosures.

### Topical Penetration Enhancers

In some embodiments, the topical formulations described herein comprise a topical penetration enhancer. The delivery of drugs topically to the skin provides many advantages. For the patient, it is comfortable, convenient, and noninvasive. The variable rates of absorption and metabolism possibly encountered in oral treatment are avoided, and other inherent inconveniences (e.g., gastrointestinal irritation, the need for administration with food in some cases or without food in other cases) are eliminated. Such localized treatment avoids incurring high systemic drug levels and possible adverse effects that could follow, i.e. inhibition of cytokine release or NF-κB activity in other biological processes.

The topical delivery of drugs into the skin, however, is commonly challenging. Skin is a structurally complex, relatively thick membrane. Molecules moving from the environment into and through intact skin must first penetrate the stratum corneum and any material on its surface. The stratum corneum is a layer approximately 10-15 micrometers thick over most of the body that consists of dense, highly keratinized cells. The high degree of keratinization within these cells, as well as their dense packing, are believed to be the factors most responsible for creating, in most cases, a substantially impermeable barrier to drug penetration. With many drugs, the rate of penetration through the skin is extremely low without the use of some means to enhance the skids permeability. As the stratum corneum of many inflammatory dermatoses is commonly thicker than that of normal skin, the penetration of topical drugs into the affected areas of skin is particularly difficult to achieve.

In order to increase the degree and rate at which a drug penetrates the skin, various approaches have been followed, each of which involves the use of either a chemical penetration enhancer or a physical penetration enhancer. Physical enhancements of skin permeation include, for example, electrophoretic techniques such as iontophoresis. The use of ultrasound (or “phonophoresis”) as a physical penetration enhancer has also been researched. Chemical penetration enhancers are more commonly used. These are compounds that are topically administered along with a drug (or, in some cases, prior to drug administration) in order to increase the permeability of the stratum corneum, and thereby provide for enhanced penetration of the drug through the skin. Ideally, such chemical penetration enhancers (or “permeation enhancers,” as the compounds are referred to herein) are compounds that are innocuous and serve merely to facilitate diffusion of the drug through the stratum corneum.

Various compounds for enhancing the permeability of skin are known in the art and are described in the pertinent texts and literature. Compounds that have been used to enhance skin permeability include: sulfoxides such as dimethylsulfoxide (DMSO) and decylmethylsulfoxide (C₁₀MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); the 1-substituted azacycloheptan-2-ones, particularly 1-n-dodecylcyclazacycloheptan-2-one (available under the trademark Azone® from Nelson Research & Development Co., Irvine, Calif.; see U.S. Pat. Nos. 3,989,816, 4,316,893, 4,405,616, and 4,557,934); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid, and succinic acid. The book Percutaneous Penetration Enhancers (Smith et al., editors, CRC Press, 1995) provides an excellent overview of the field and further background information on a number of chemical and physical enhancers.

It has long been thought that strong bases, such as NaOH, were not suitable as permeation enhancers because they would damage skin. It has been now been discovered that the skin permeability of various drugs could be enhanced without skin damage by exposing the skin to a base or basic solution, in a skin contacting formulation or patch. The desired pH of the solution on the skin can be obtained using a variety of bases or base concentrations. Accordingly, the pH is selected so as to be low enough so as to not cause skin damage, but high enough to enhance skin permeation to various active agents. As such, it is important that the amount of base in any patch or formulation is optimized so as to increase the flux of the drug through the body surface while minimizing any possibility of skin damage. In some embodiments, this means that the pH at the body surface in contact with a formulation or drug delivery system of the invention is in the range of approximately pH 8.0 to about pH 13.0, about pH 8.0 to about pH 11.5, about pH 8.5 to about pH 11.5, or about pH 8.5 to about pH 10.5. In some embodiments, the pH is in the range of about pH 9.5 to about pH 11.5, or about pH 10.0 to about pH 11.5.

In one embodiment, the pH at the skin surface is the primary design consideration, i.e., the composition or system is designed so as to provide the desired pH at the skin surface. In certain instances, anhydrous formulations and transdermal systems do not have a measurable pH, and the formulation or system is designed so as to provide a target pH at the skin surface. Moisture from the body surface can migrate into the formulation or system, dissolve the base and thus release the base into solution, which will then provide the desired target pH at body surface. In certain instances, a hydrophilic composition is desirable. In addition, when using aqueous formulations, the pH of the formulation in certain instances changes over time after it is applied on the skin. For example, gels, solutions, ointments, etc., in certain instances, experience a net loss of moisture after being applied to the body surface, i.e., the amount of water lost is greater than the amount of water received from the body surface. In that case, the pH of the formulation in certain instance is different than its pH when manufactured. In some embodiments, this problem is easily remedied by designing the aqueous formulations to provide a target pH at the body surface.

In other embodiments, the pH of the formulation or the drug composition contained within a delivery system will be in the range of approximately pH 8.0 to about pH 13.0, about pH 8.0 to about pH 11.5, about pH 8.5 to about pH 11.5, or about pH 8.5 to about pH 10.5. In some embodiments, the pH will be in the range of about pH 9.5 to about pH 11.5, or about pH 10.0 to about pH 11.5. In one embodiment of the invention the pH of the formulation is higher than the pH at the body surface. For example, if an aqueous formulation is used, moisture from the body surface can dilute the formulation, and thus provide for a different pH at the body surface, which will typically be lower than that of the formulation itself.

In one embodiment, the body surface is exposed to a base or basic solution for a sufficient period of time so as to provide a high pH at the skin surface, thus creating channels in the skin or mucosa for the drug to go through. It is expected that drug flux is proportional to the strength of the solution and the duration of exposure. However, it is desirable to balance the maximization of drug flux with the minimization of skin damage. This can be done in numerous ways. For example, in some embodiments, the skin damage is minimized by selecting a lower pH within the 8.0 to 13.0 range, by exposing the skin to the formulation or system for a shorter period of time, or by including at least one irritation-mitigating additive. Alternatively, the patient can be advised to change the location of application with each subsequent administration.

While certain amounts are set forth below, it is understood that, for all of the inorganic and organic bases described herein, the optimum amount of any such base will depend on the strength or weakness of the base and its molecular weight, and other factors such as the number of ionizable sites in the active agent being administered and whether there are any acidic species present in the formulation or patch. One skilled in the art can readily determine the optimum amount for any particular base such that the degree of enhancement is optimized while the possibility of damage to the body surface is eliminated or at least substantially minimized

Exemplary inorganic bases are inorganic hydroxides, inorganic oxides, inorganic salts of weak acids, and combinations thereof. Some inorganic bases are those whose aqueous solutions have a high pH, and are acceptable as food or pharmaceutical additives. Examples of such inorganic bases include ammonium hydroxide, sodium hydroxide, potassium hydroxide, calcium hydroxide, magnesium hydroxide, magnesium oxide, calcium oxide, Ca(OH)₂, sodium acetate, sodium borate, sodium metaborate, sodium carbonate, sodium bicarbonate, sodium phosphate, potassium carbonate, potassium bicarbonate, potassium citrate, potassium acetate, potassium phosphate and ammonium phosphate and combinations thereof.

Inorganic hydroxides include, for example, ammonium hydroxide, alkali metal hydroxide and alkaline earth metal hydroxides, and mixtures thereof. Some inorganic hydroxides include ammonium hydroxide; monovalent alkali metal hydroxides such as sodium hydroxide and potassium hydroxide; divalent alkali earth metal hydroxides such as calcium hydroxide and magnesium hydroxide; and combinations thereof.

The amount of inorganic hydroxide included in the compositions and systems of the invention will typically represent about 0.3-7.0 W/V %, about 0.5-4.0 W/V %, about 0.5-3.0 W/V %, or about 0.75-2.0 W/V % of a topically applied formulation or of a drug reservoir of a drug delivery system, or patch.

Inorganic oxides include, for example, magnesium oxide, calcium oxide, and the like.

In some embodiments, the amount of inorganic oxide included in the compositions and systems of the invention is substantially higher than the numbers set forth above for the inorganic hydroxide. In some instance, it is as high as 20 wt %, in some cases as high as 25 wt % or higher, but will generally be in the range of about 2-20 wt %. In some embodiments, these amounts are adjusted to take into consideration the presence of any base-neutralizable species.

Inorganic salts of weak acids include, ammonium phosphate (dibasic); alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic); alkaline earth metal salts of weak acids such as magnesium phosphate and calcium phosphate; and the like, and combinations thereof.

Organic bases suitable for use in the invention are compounds having an amino group, amido group, an oxime, a cyano group, an aromatic or non-aromatic nitrogen-containing heterocycle, a urea group, and combinations thereof. More specifically, examples of suitable organic bases are nitrogenous bases, which include, but are not limited to, primary amines, secondary amines, tertiary amines, amidines, guanidines, hydroxylamines, cyano guanidines, cyanoamidines, oximes, cyano (—CN) containing groups, aromatic and non-aromatic nitrogen-containing heterocycles, urea, and mixtures thereof. In some embodiments, the organic bases are primary amines, secondary amines, tertiary amines, aromatic and non-aromatic nitrogen-containing heterocycles, and mixtures thereof.

For all permeation-enhancing bases herein, the optimum amount of any particular agent will depend on the strength or weakness of the base, the molecular weight of the base, and other factors such as the number of ionizable sites in the drug administered and any other acidic species in the formulation or patch. One skilled in the art can readily determine the optimum amount for any particular agent by ensuring that a formulation is effective to provide a pH at the skin surface, upon application of the formulation, in the range of about pH 7.5 to about pH 13.0, about pH 8.0 to about pH 11.5, or about pH 8.5 to about pH 10.5. In some embodiments, the pH will be in the range of about pH 9.5 to about pH 11.5, or about pH 10.0 to about pH 11.5. This in turn ensures that the degree of treatment is maximized while the possibility of damage to the body surface is eliminated or at least substantially minimized

In the case of intranasal administration, such solutions or suspensions, in some embodiments, are isotonic relative to nasal secretions and of about the same pH, ranging e.g., from about pH 4.0 to about pH 7.4 or from about pH 6.0 to about pH 7.0. Buffers should be physiologically compatible and include, simply by way of example, phosphate buffers. For example, a representative nasal decongestant is described as being buffered to a pH of about 6.2 (Remington's Pharmaceutical Sciences 16th edition, Ed. Arthur Osol, page 1445 (1980)). One skilled in the art can readily determine a suitable saline content and pH for an innocuous aqueous solution for nasal and/or upper respiratory administration. An example of a suitable formulation for intranasal administration, is an aqueous solution buffered to a pH of about 6.0 to about 8.0 with Sodium Phosphate, Monobasic, comprising about 1% W/V of the LFA-1 antagonist, up to about 0.1% W/V EDTA, and, optionally, up to about 0.4% w/w Methylparaben and up to about 0.02% w/w Propylparaben.

Additional permeation enhancers will be known to those of ordinary skill in the art of topical drug delivery, and/or are described in the pertinent texts and literature. See, e.g., Percutaneous Penetration Enhancers, Smith et al., eds. (CRC Press, 1995).

Disclosed herein, in certain embodiments, is a topical formulation of a chemerin C15 peptide wherein the topical formulation comprises a penetration enhancer. Penetration enhancers include, but are not limited to, sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween 80, nonylphenoxypolyethylene (NP-POE), polysorbates, sodium glycocholate, sodium deoxycholate, sodium taurocholate, sodium taurodihydrofusidate, sodium glycodihydrofusidate, oleic acid, caprylic acid, mono- and di-glycerides, lauric acids, acylcholines, caprylic acids, acylcarnitines, sodium caprates, EDTA, citric acid, salicylates, DMSO, decylmethyl sulfoxide, ethanol, isopropanol, propylene glycol, polyethylene glycol, glycerol, propanediol, and diethylene glycol monoethyl ether. In some embodiments, the topical formulation of a chemerin C15 contains a penetration enhancer. In some embodiments, the topical formulation of a chemerin C15 does not contain a penetration enhancer. In some embodiments, the topical formulation of a chemerin C15 contains DMSO. In some embodiments, the topical formulation of a chemerin C15 does not contain DMSO.

### Combination Therapies

In some embodiments, the topical formulation comprises at least one additional therapeutic agent in addition to the chemerin C15 peptide. In some embodiments, the additional therapeutic agent is an antioxidant, anti-inflammatory agent, antimicrobial agent, antiangiogenic agent, anti-apoptotic agent, vascular endothelial growth factor inhibitor, antiviral agent, calcineurin inhibitor, corticosteroid, or immunomodulator. In some embodiments, the topical formulation comprising a chemerin C15 peptide is a corticosteroid. In some embodiments, the corticosteroid is a topical corticosteroid. Agents for use with the chemerin C15 peptides are further described in the Combination Therapies section herein.

### Administration and Dosages

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide.

The benefits of topical administration include localized delivery of the therapeutic agent directly to the affected tissue and minimal systemic side effects due to low systemic bioavailability. For example, in some embodiments, topical formulations provided herein are administered directly to the skin, eye, mouth, nose, vaginal mucosa or anal mucosa. The methods of topical delivery provided herein are particularly well suited for localized administration of the formulation. Suitable formulations and additional carriers are discussed herein and, additionally, described in Remington “The Science and Practice of Pharmacy” (20.sup.th Ed., Lippincott Williams & Wilkins, Baltimore Md.), the teachings of which are incorporated by reference in their entirety herein.

One advantage of the therapeutic composition according to the invention is that topical application is particularly convenient for treating and preventing a variety of dermal conditions. In some embodiments, therapeutic compositions are noninvasively applied directly to the site of interest. Other disorders conveniently addressed by topical administration include allergic conditions of the nasal passageway, eye, and oral cavity. In some embodiments, chemerin C15 peptides provided have a rapid systemic clearance such that any drug that gets absorbed systemically is quickly cleared.

In some embodiments, the local concentration of the chemerin C15 peptide is about 2 times, 3 times, 4 times, 5 times, 10 times, 25 times, 50 times, or 100 times greater than the systemic concentration. In another embodiment, local concentration of chemerin C15 peptide is 100 times greater than the systemic concentration. In another embodiment, local concentration of chemerin C15 peptide is 1000 times greater than the systemic concentration. In one embodiment, the local concentration is about 10,000 times or more greater than the systemic concentration at the same time point. In some embodiments, the concentration of therapeutic agent is measured using any known method in the art (e.g. ELISA and/or LCMS/MS).

In certain instances, the method of delivery of the pharmaceutically active composition selected involves application of a formulation of the invention to an area of body surface affected with an inflammatory or immune related condition or symptom thereof. In embodiments of the methods provided, the formulation is topically applied to skin, eyes, mouth, nose, vaginal mucosa or anal mucosa. In some embodiments, a cream, ointment, paste, plaster, or lotion is spread on the affected area of skin and gently rubbed in. In some embodiments, a polymeric or other bioadhesive formulation is spread or dabbed on the affected area of skin. In some embodiments, a solution is applied in the same ways, but more typically will be applied with a dropper, spray, swab, or the like, and carefully applied to the affected area of skin. In some embodiments, petrolatum is spread on the skin surrounding the affected area of skin to protect it from possible irritation during treatment.

In some embodiments, topical delivery is achieved by use of a delivery device that facilitates the delivery of the agent directly into the skin tissue, e.g. micro-needle injection devices, or a delivery device comprised of a covering for the skin whereby the agent is held between the affected skin and covering for prolonged periods by means of an adhesive property of the covering.

### Dosing

Disclosed herein, in certain embodiments, is a topical formulation of a chemerin C15 peptide wherein the topical formulation administered for prophylactic and/or therapeutic treatments. In certain instances, amounts effective for this use will depend on the severity and course of the disease, disorder or condition, previous therapy, the individual's health status and response to the drugs, and the judgment of the treating physician

The compositions are delivered with a pharmacokinetic profile that results in the delivery of an effective dose of the chemerin C15 peptide. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (e.g. by means of an appropriate, conventional pharmacological protocol). The total daily doses of the medicaments contemplated for administration, and consequently the concentrations by weight of the medicaments in the respective compositions, can vary widely, but are within the typical skill of the routine practitioner.

In some embodiments, a topical formulation of a chemerin C15 peptide is delivered such that a local therapeutically effective concentration is achieved. For example, in some embodiments, the local therapeutically effective concentration is achieved with a local tissue concentration of the chemerin C15 peptide sufficient to inhibit cellular process associated with inflammation by at least about 10%, 20%, 30%, 40%, 50%, 60%, 70%, 80%, 90% in an in vitro dose titration study. In some embodiments, the local therapeutically effective concentration is achieved with a local tissue concentration of the chemerin C15 peptide sufficient to inhibit cellular process associated with inflammation by at least about 50% in an in vitro dose titration study. For example, in some embodiments, the local therapeutically effective concentration is achieved with a local tissue concentration of the chemerin C15 peptide sufficient to inhibit cellular process associated with inflammation by at least about 10%, 20%, 30%, 40%, 50%, 60%, 70%, 80%, 90% in vitro in an antigen presenting cell, such as a macrophage or a dendritic cell. In some embodiments, the local therapeutically effective concentration is achieved with a local tissue concentration of the chemerin C15 peptide sufficient to inhibit cellular process associated with inflammation by at least about 50% in vitro in an antigen presenting cell, such as a macrophage or a dendritic cell. In some embodiments, the antigen presenting cell is stimulated, such as, for example, by contacting the cell with IFNγ and/or LPS prior to, during or following addition of the chemerin C15 peptide.

In some embodiments, the local therapeutically effective concentration is achieved with a local tissue concentration of the chemerin C15 peptide sufficient to inhibit secretion of one or more inflammatory cytokines by at least about 10%, 20%, 30%, 40%, 50%, 60%, 70%, 80%, 90% in vitro in an antigen presenting cell, such as a macrophage or a dendritic cell. In some embodiments, the local therapeutically effective concentration is achieved with a local tissue concentration of the chemerin C15 peptide sufficient to inhibit secretion of one or more inflammatory cytokines by at least about 50% in vitro in an antigen presenting cell, such as a macrophage or a dendritic cell. In some embodiments, the antigen presenting cell is stimulated, such as, for example, by contacting the cell with IFNγ and/or LPS. In some embodiments, the antigen presenting cell is stimulated, such as, for example, by contacting the cell with IFNγ and/or LPS prior to, during or following addition of the chemerin C15 peptide.

In some embodiments, the local therapeutically effective concentration is achieved with a local tissue concentration of the chemerin C15 peptide sufficient to inhibit transcription of one or more inflammatory cytokines by at least about 10%, 20%, 30%, 40%, 50%, 60%, 70%, 80%, 90% in vitro in an antigen presenting cell, such as a macrophage or a dendritic cell. In some embodiments, the local therapeutically effective concentration is achieved with a local tissue concentration of the chemerin C15 peptide sufficient to inhibit transcription of one or more inflammatory cytokines by at least about 50% in vitro in an antigen presenting cell, such as a macrophage or a dendritic cell. In some embodiments, the antigen presenting cell is stimulated, such as, for example, by contacting the cell with IFNγ and/or LPS prior to, during or following addition of the chemerin C15 peptide. In some embodiments, the inflammatory cytokine is IL-23, IL-12, TNFα, IL-1β, IL-6, or RANTES.

In some embodiments, the local therapeutically effective concentration is achieved with a local tissue concentration of the chemerin C15 peptide of greater than about 0.1 pM-100 nM. In some embodiments, the local therapeutically effective concentration is achieved with a local tissue concentration of the chemerin C15 peptide of greater than about 1 pM-10 nM. In some embodiments, the local therapeutically effective concentration is achieved with a local tissue concentration of the chemerin C15 peptide of greater than about 1 pM-1 nM. In some embodiments, the local therapeutically effective concentration is achieved with a local tissue concentration of the chemerin C15 peptide of greater than about 1-100 pM. In some embodiments, the local therapeutically effective concentration is achieved with a local tissue concentration of the chemerin C15 peptide of greater than about 1-10 pM. In some embodiments, chemerin C15 peptide achieves a local tissue concentration of greater than about 1 nM within about 1-12 hours following administration to a subject. In some embodiments, chemerin C15 peptide achieves a local tissue concentration of greater than about 10 pM within about 1-12 hours following administration to a subject. In some embodiments, chemerin C15 peptide achieves a local tissue concentration of greater than about 10 pM within about 1-12 hours following administration to a subject. In some embodiments, chemerin C15 peptide achieves a local tissue concentration of greater than about 1 pM within about 1-12 hours following administration to a subject.

In some embodiments, the local therapeutically effective concentration of the chemerin C15 peptide is achieved while maintaining a low systemic level. For example, in some embodiments, a local therapeutically effective concentration of about 1 pM-10 nM is achieved while maintaining a systemic drug concentration of less than 1-100 pM. For example, in some embodiments, a local therapeutically effective concentration of about 1 pM-1 nM is achieved while maintaining a systemic drug concentration of less than 1-100 pM. For example, in some embodiments, a local therapeutically effective concentration of about 1-100 pM is achieved while maintaining a systemic drug concentration of less than 1-100 pM.

For example, in some embodiments, a local therapeutically effective concentration of about 1 pM-10 nM is achieved while maintaining a systemic drug concentration of less than 10-100 pM. For example, in some embodiments, a local therapeutically effective concentration of about 1 pM-1 nM is achieved while maintaining a systemic drug concentration of less than 10-100 pM. For example, in some embodiments, a local therapeutically effective concentration of about 1-100 pM is achieved while maintaining a systemic drug concentration of less than 10-100 pM.

In other embodiments, a local therapeutically effective concentration of about 1 pM-10 nM is achieved while maintaining a systemic drug concentration of less than 1000 pM. In other embodiments, a local therapeutically effective concentration of about 1 pM-10 nM is achieved while maintaining a systemic drug concentration of less than 10 pM. In other embodiments, a local therapeutically effective concentration of about 1 pM-1 nM is achieved while maintaining a systemic drug concentration of less than 1000 pM. In other embodiments, a local therapeutically effective concentration of about 1 pM-1 nM is achieved while maintaining a systemic drug concentration of less than 10 pM. In other embodiments, a local therapeutically effective concentration of about 1-100 pM is achieved while maintaining a systemic drug concentration of less than 1000 pM. In other embodiments, a local therapeutically effective concentration of about 1-100 pM is achieved while maintaining a systemic drug concentration of less than 10 pM.

In some embodiments, the systemic concentration of the peptide is measured by blood plasma concentration using any of a variety of methods known in the art and as disclosed above, such as for example an ELISA and/or LCMS/MS.

In some embodiments, an effective amount of the chemerin C15 peptide is a dose of about 0.01-100 milligrams per square inch. In some embodiments, an effective amount of the chemerin C15 peptide is a dose of about 0.01-10 milligrams per square inch. In some embodiments, an effective amount of the chemerin C15 peptide is a dose of about 0.1-100 milligrams per square inch. In some embodiments, an effective amount of the chemerin C15 peptide is a dose of about 0.1-10 milligrams per square inch.

In some embodiments, the dosing regimen depends on a number of factors that are readily be determined, such as the size of the affected area, the severity of the dermatosis, and the responsiveness of the inflammatory dermatosis to treatment, but will normally be one or more doses per day, with a course of treatment lasting from several days to several months, or until a cure is effected or a significant diminution in the size and/or severity of the inflammatory dermatosis is achieved. In some embodiments, another dosing regimen favors the use of a systemic biologic agent and/or potent topical agent to cure or significantly diminish the size and/or severity of the inflammatory dermatosis and then dose the site of the dermatosis with chemerin C15 peptide to prevent remission or return of the dermatosis. Local administration of topical formulation of a chemerin C15 peptide that is rapidly cleared from the systemic circulation has a particular benefit for patients with inflammatory diseases affecting large areas. In some embodiments, patients are able to treat large areas without significant immunosuppression and risk of side effects due to systemic exposure to drug. One of ordinary skill can readily-determine optimum dosages, dosing methodologies, and repetition rates. In general, it is contemplated that the formulation will be applied one to four times daily. With a skin patch, the device is generally maintained in place on the body surface throughout a drug delivery period, typically in the range of 8 to 72 hours, and replaced as necessary.

In some embodiments, the topical formulation of a chemerin C15 peptide is present in an amount sufficient to exert a therapeutic effect to reduce symptoms of an immune related or inflammatory disease or disorder by an average of at least about 5, 10, 15, 20, 25, 30, 40, 50, 60, 70, 80, 90, more than 90%, or substantially eliminate symptoms of the immune related or inflammatory disease or disorder. For many inflammatory diseases, there are well recognized clinical assessments of therapeutic effect (e.g. PASI and/or PGA score for psoriasis and EASI score for eczema)

In some embodiments, the topical formulation of a chemerin C15 peptide is administered in a single dose. In some embodiments, a single dose of a chemerin C15 peptide is administered for treatment of an acute condition. In some embodiments, a single dose of a chemerin C15 peptide is administered is used when it is co-administered with an additional therapeutic agent for treatment of an acute condition.

In some embodiments, the topical formulation of a chemerin C15 peptide (by itself or in combination with one or more additional therapeutic agents) is administered in multiple doses. In some embodiments, dosing is about once, twice, three times, four times, five times, six times, seven times, eight times, nine times, ten times or more than ten times per day. In some embodiments, dosing is about once a year, twice a year, every six months, every 4 months, every 3 months, every 60 days, once a month, once every two weeks, once a week, or once every other day.

In some embodiments, the topical formulation of a chemerin C15 peptide and another therapeutic agent are administered together about once per day to about 10 times per day. In another embodiment, an additional therapeutic agent is administered concurrent with, prior to, or subsequent to administering the topical formulation of a chemerin C15 peptide. In another embodiment the administration of the topical formulation of a chemerin C15 peptide and another therapeutic agent continues for less than about 7 days. In yet another embodiment the co-administration continues for more than about 6, 10, 14, 28 days, two months, six months, or one year. In some cases, co-administered dosing is maintained as long as necessary, e.g., dosing for chronic inflammation.

In some embodiments, a topical formulation of a chemerin C15 peptide is administered once per day. In some embodiments, a topical formulation of a chemerin C15 peptide is administered twice per day. In some embodiments, a topical formulation of a chemerin C15 peptide is administered three times per day. In some embodiments, a topical formulation of a chemerin C15 peptide is administered any time. In some embodiments, a topical formulation of a chemerin C15 peptide is administered in the morning. In some embodiments, a topical formulation of a chemerin C15 peptide is administered during the day. In some embodiments, a topical formulation of a chemerin C15 peptide is administered in the evening. In some embodiments, a topical formulation of a chemerin C15 peptide is administered at night.

In another aspect of the invention, the local tissue concentration of the chemerin C15 peptide is maintained at therapeutically effective levels for an extended period of time. In some embodiments, the local tissue concentrations of the chemerin C15 peptide is maintained at therapeutically effective levels for a certain amount of time or between doses. In some examples, a chemerin C15 peptide selected for local administration maintains local therapeutically effective levels for extended periods such the subject achieves a therapeutic effect without administration of multiple doses per day.

In some embodiments, the chemerin C15 peptide has a local tissue concentration of greater than about 1-1000 pM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject. In some embodiments, the chemerin C15 peptide has a local tissue concentration of greater than about 1-100 pM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject. In some embodiments, the chemerin C15 peptide has a local tissue concentration of greater than about 1-100 pM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject. In some embodiments, the chemerin C15 peptide has a local tissue concentration of greater than about 10-100 pM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject. In some embodiments, the chemerin C15 peptide has a local tissue concentration of greater than about 1-10 pM for at least about 2 hours, about 4 hours, about 6 hours, about 8 hours, about 10 hours, about 12 hours, about 14 hours, about 16 hours, about 18 hours, about 20 hours, about 22 hours, or about 24 hours following administration to a subject.

In some embodiments, administration of the topical formulation continues as long as necessary to treat the disease or disorder. In some embodiments, a composition of the invention is administered for more than 1, 2, 3, 4, 5, 6, 7, 14, or 28 days. In some embodiments, a composition of the invention is administered for less than 28, 14, 7, 6, 5, 4, 3, 2, or 1 day. In some embodiments, a composition of the invention is administered chronically on an ongoing basis, e.g., for the treatment of chronic inflammation.

In some embodiments, where a dermatological disorder does not improve, a topical formulation disclosed herein is administered chronically (i.e., for an extended period of time, including throughout the duration of the individual's life). In some embodiments, where a dermatological disorder does improve, a topical formulation disclosed herein is given continuously. In some embodiments, the dose of active agent being administered is temporarily reduced or temporarily suspended for a certain length of time (i.e., a “drug holiday”). In some embodiments, a drug holiday lasts between 2 days and 1 year, including all integers in between. In some embodiments, the dose reduction during a drug holiday is from about 10% to about 100%, including all integers in between.

In some embodiments, where a dermatological disorder does improve, a topical formulation disclosed herein is administered as a maintenance dose. In some embodiments, where a dermatological disorder does improve, a topical formulation disclosed herein is administered with reduced frequency or at a reduced dose.

In some embodiments, a topical formulation disclosed herein is formulated for controlled release of a chemerin C15 peptide. In some embodiments, a chemerin C15 peptide is released over a time period exceeding 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

### Combination Therapies

Disclosed herein, in certain embodiments, are chemerin C15 peptides. Further disclosed herein are topical formulations comprising a chemerin C15 peptide and optionally a pharmaceutically acceptable excipient. Additionally disclosed herein are methods of treating inflammatory dermatological disorders in an individual in need thereof comprising administering a chemerin C15 peptide disclosed herein or a topical formulation comprising a chemerin C15 peptide disclosed herein. Further disclosed herein are methods of inhibiting the activity of an inflammatory cytokine or chemokine in an individual in need thereof comprising administering a chemerin C15 peptide disclosed herein or a topical formulation comprising a chemerin C15 peptide disclosed herein. Also disclosed herein, in certain embodiments, are method of inhibiting inhibits nuclear translocation or NFκB-mediated gene transcription of an inflammatory cytokine in an individual in need thereof comprising administering a chemerin C15 peptide disclosed herein or a topical formulation comprising a chemerin C15 peptide disclosed herein. In some embodiments, the chemerin C15 peptide is a human chemerin C15 peptide. In some embodiments, the chemerin C15 peptide is a salt of a chemerin C15 peptide. In some embodiments, the chemerin C15 peptide is carboxylated. In some embodiments, the chemerin C15 peptide is amidated. In some embodiments, the chemerin C15 peptide is cyclic. In some embodiments, the chemerin C15 peptide is at least 80%, 85%, 90%, 91%, 92%, 93%, 94%, 95%, 96%, 97%, 98%, 99%, 99.5%, 99.6%, 99.7%, 99.8%, or 99.9% homologous to a naturally occurring chemerin C15 peptide. In some embodiments, the aforementioned methods or formulations further comprise an additional therapeutic agent.

In some embodiments, the additional therapeutic agent treats the inflammatory dermatological disorder. In some embodiments, the additional therapeutic agent modulates side-effects of the chemerin C15 peptide. In some instances, pathological events in this disease state are marked by a combination of impaired autoregulation, apoptosis, ischemia, neovascularization, and inflammatory stimuli. In some embodiments, the combination of a chemerin C15 peptide and an additional therapeutic produces additive or synergistic effects.

In some embodiments, the additional therapeutic agent is an antioxidant, antiinflammatory agent, antimicrobial including antibacterial, antihistamine, mast cell stabilizer, antiviral and antifungal agents, antiangiogenic agent, anti-apoptotic agent, lubricant, and/or secretagogue.

Inflammation is induced by the process of leukocyte adhesion and neovascularization. In some embodiments, anti-inflammatory agents are administered in combination, prior to, after, or concomitantly with a chemerin C15 peptide. In some embodiments, the anti-inflammatory agents are chosen from corticosteroid related drugs including, but not limited to, adexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene, prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, deacylcortivazol, and the like. In some embodiments, the anti-inflammatory agents are chosen from 5-aminosalicylate (5-ASA) compounds, such as sulfasalzine (Azulfidine), osalazine (Dipentum), and mesalamine (examples include Pentasa, Asacol, Dipentum, Colazal, Rowasa enema, and Canasa suppository). In some embodiments, the anti-inflammatory agents are chosen from cyclosporine related drugs (e.g. calcineurin antagonist) including but not limited to members of the cyclosporine family, and other related calcineurin antagonists including sirolimus, tacorlimus and pimecrolimus. In some embodiments, the anti-inflammatory agents are chosen from the group of NSAIDs including but not limited to acetaminophen, acemetacin, aceclofenac, alminoprofen, amfenac, bendazac, benoxaprofen, bromfenac, bucloxic acid, butibufen, carprofen, celecoxib, cinmetacin, clopirac, diclofenac, etodolac, etoricoxib, felbinac, fenclozic acid, fenbufen, fenoprofen, fentiazac, flunoxaprofen, flurbiprofen, ibufenac, ibuprofen, indomethacin, isofezolac, isoxicam, isoxepac, indoprofen, ketoprofen, lonazolac, loxoprofen, mefenamic acid, meclofenamic acid, meloxicam, metiazinic acid, mofezolac, miroprofen, naproxen, niflumic, oxaprozin, pirozolac, pirprofen, pranoprofen, protizinic acid, rofecoxib, salicylic acid and its derivatives (i.e. for example, aspirin), sulindac, suprofen, suxibuzone, triaprofenic acid, tolmetin, valdecoxib, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, mesalamine, prodrugs thereof, and the like. In some embodiments, immunomodulators such as 6-mercaptopurine (6-MP), azathioprine (Imuran), methotrexate (Rheumatrex, Trexall), Stelara, infliximab (Remicade), and adalimumab (Humira) are used.

In some embodiments, the additional therapeutic agent is a Vascular Endothelial Growth Factor (VEGF) inhibitor such as, for example 1) neutralizing monoclonal antibodies against VEGF or its receptor, 2) small molecule tyrosine kinase inhibitors of VEGF receptors, 3) soluble VEGF receptors which act as decoy receptors for VEGF, and 4) ribozymes which specifically target VEGF. Some examples of antibodies which are active against VEGF are, for example, Lucentis (ranibizumab), and Avastin (bevacizumab). An example of an oligonucleotide drug is, e.g., Macugen (pegaptanib sodium injection). Small molecule tyrosine kinase inhibitors include, for example, pazopanib, sorafenib, sutent, and the like.

A class of therapeutic agents useful for administration in combination, prior to, after, or concomitantly with a chemerin C15 peptide are antihistamines, including alkylamine, ethanolamine and phenothiazine classes, such as, for example, chlorpheniramine maleate, chlorphenamiramine tannate, diphenhydramine hydrochloride, promethazine hydrochloride, acrivastine, azatadine maleate, azelastine hydrochloride, brompheniramine maleate, carbinoxamine maleate, cetirizine hydrochloride, clemastine fumarate, cyproheptadine hydrochloride, desloratadine, dexbrompheniramine maleate, dexchlorpheniramine maleate, dimenhydriunate, diphenhydramine hydrochloride, emedastine difumarate, fexofenadine hydrochloride, hydroxyzine hydrochloride, ketotifen fumarate, loratadine, meclizine hydrochloride, olopatadine hydrochloride, phenindamine tartrate, quetiapine, tripelennamine citrate, tripelennamine hydrochloride, and triprolidine hydrochloride.

A class of therapeutic agents useful for administration in combination, prior to, after, or concomitantly with a chemerin C15 peptide are mast cell stabilizers such as cromolyn sodium and nedocromil.

Oxidative stress, in certain instances, is induced in cells with impaired autoregulatory and ischemic processes induced by immune or inflammatory disorders. In some embodiments, anti-oxidants useful for administration in combination, prior to, after, or concomitantly with a chemerin C15 peptide. Examples of suitable anti-oxidants useful in the methods of the invention include, but are not limited to, ascorbic acid, tocopherols, tocotrienols, carotinoids, glutathione, alpha-lipoic acid, ubiquinols, bioflavonoids, carnitine, and superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like.

In some embodiments, methods are provided wherein anti-apoptotic therapeutic agents are administered in combination, prior to, after, or concomitantly with a chemerin C15 peptide. Examples of suitable anti-apoptotic agents are, for example, inhibitors of caspases, cathepsins, and TNF-α.

A class of therapeutic agents useful for administration in combination, prior to, after, or concomitantly with a chemerin C15 peptide are antimicrobial agents. Suitable antimicrobial compounds, include, but are not limited to, penicillins, such as, for example, amoxicillin, ampicillin, azlocillin, carbenicillin, cloxacillin, dicloxacillin, flucloxacillin, mezlocillin, nafcillin, penicillin, piperacillin, ticarcillin, and the like; beta-lactamase inhibitors; carbapenems, such as, for example, ertapenem, imipenem, meropenem, and the like; cephalosporins, such as, for example, cefaclor, cefamandole, cefoxitin, cefprozil, ceftiroxime, cefixime, cefdinir, cefditoren, cefoperazone, cefotaxime, cefpodoxime, cefadroxil, ceftazidime, ceftibuten, ceftizoxime, ceffiriaxone, cefazolin, cefixime, cephalexin, cefepime, and the like; quinolones, such as, for example, ciprofloxacin, enoxacin, gatifloxacin, levofloxacin, lomefloxacin, morifloxacin, norfloxacin, ofloxacin, trovafloxacin, and the like; macrolides, such as, for example, azithromycin, clarithromycin, dirithromycin, erythromycin, milbemycin, troleandomycin, and the like; monbactams, such as, for example, an LFA-1 antagonist, and the like; tetracyclins, such as, for example, demeclocyclin, doxycycline, minocycline, oxytetracyclin, tetracycline, and the like; aminoglycosides, such as, for example, amikacin, gentamicin, kanamycin, neomycin, netilmicin, paromomycin, streptomycin, tobramycin, and the like; carbacephem, such as, for example, loracarbef, and the like; streptogramins; sulfonamides, such as, for example, mefanide, prontosil, sulfacetamide, sulfamethizole, sulfanilamide, sulfasalazine, sulfisoxazole, trimethoprim, trimethoprim-sultamethoxazole, and the like; other antimicrobials such as metronidazole; and the combination drugs such as for example, sulfamethoxazole and trimethoprim, and the like.

Other antimicrobial agents include the class of antiviral agents. Antiviral agents include, but are not limited to therapeutic agents such as entry inhibitors, reverse transcriptase inhibitors, nucleoside or nucleotide analogs, protease inhibitors, and inhibitors of viral release from host cells. Some illustrative therapeutic agents of this group, include, but are not limited to abacavir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, famciclovir, fomivirsen, foscarnet, fosfonet, ganciclovir, gardasil, ibacitabine, immunovir, idoxuridine, imiquimod, indinavir, inosine, interferon type III, interferon type II, interferon type I, interferon, lamivudine, lopinavir, loviride, maraviroc, moroxydine, nelfinavir, neviapine, nexavir, oseltamivir, penciclovir, peramivir, pleconaril, podophyllotoxin, raltegravir, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, and the like.

In some of the embodiments, the formulations administered to the skin comprise one or more antimicrobial or antibiotic agents.

In some of the embodiments, secretagogues are administered in combination, prior to, concomitantly with, or subsequent to administration of a chemerin C15 peptide. In some embodiments, increasing mucin or other fluid production in the eye is beneficial. Examples include but are not limited to Diquafasol, Rebamipide, and Eicosanoid 15-(S)-HETE.

### EXAMPLES

The following examples are illustrative and non-limiting to the scope of the formulations and methods described herein.

### Example 1

### Effect of hC-15 on Cytokine Secretion by Human Macrophages

In this example, the ability of human chemerin C15 to inhibit secretion of cytokines from activated human macrophages was examined. For this experiment, the activity of the human chemerin C15 peptide AGEDPHSFYFPGQFA (SEQ ID NO: 1) was compared to that of the human chemerin C17 peptide AGEDPHSFYFPGQFAFS (SEQ ID NO: 25). The C15 and C17 peptides were synthesized by solid phase synthesis using BOP coupling of FMOC protected amino acids with final cleavage from the resin with TFA. Peptides were purified by reverse phase C18 chromatography using a water/acetonitrile gradient.

Human macrophages were derived from human CD14+ monocytes obtained from 3 donors. On Day 1, the isolated monocytes were thawed and seeded in triplicate for each group in 1 ml RPMI 1640 GlutaMAX™ media (supplemented with 10% FBS, 100 U/ml penicillin, 100 μg/ml streptomycin, 0.05 μM mercaptoethanol, 1% NEAA and 1% sodium pyruvate) per well of a 24 well cell culture dish at a cell concentration of 5×10⁵ cells/ml. M-CSF was added to each well to give a final concentration of 25 ng/ml. Cells were grown for 7 days at 37° C. with 5% CO₂ to differentiate the cells into macrophages. Media and M-CSF were replaced after 4 days.

Following differentiation, the media containing M-CSF was removed. The cells were washed and vehicle control, dexamethasone, C15 or C17 were added to the appropriate wells. The test peptides were dissolved in 50% DMSO/water prior to addition. C-15 (MW 1669; 16.7 mg/ml) was added to a final concentration of 1 pM, 10 pM or 100 and C17 (MW 1904; 19.0 mg/ml) was added to a final concentration of 1 μM. Dexamethasone was added to a final concentration of 1 μM. Following addition to the wells, the plates were incubated at 37° C. with 5% CO₂ for 1 hour. An equal volume of complete media was added to the non-treated wells. Control or test treatments were maintained at the correct concentration throughout the assay.

IFNγ (final concentration 20 ng/ml) was then added to the appropriate wells. Following IFNγ addition, the plates were incubated at 37° C. with 5% CO₂ for 4 hours. The concentration of vehicle control, test treatments or dexamethasone was maintained during IFNγ stimulation. LPS (final concentration 10 ng/ml) was then added to the appropriate wells. Following LPS addition, the plates were incubated at 37° C. with 5% CO₂ for 15 hours. After 6 hours, ˜60 μl of culture supernatant was removed from all wells and stored at −80° C. for analysis. The concentration of vehicle control, test treatments or dexamethasone was maintained during LPS stimulation.

At 15 hours post LPS stimulation, the remaining cell culture supernatant was harvested and stored at −80° C. until assayed. The concentration of vehicle control, test treatments or dexamethasone was maintained until culture termination.

Cell culture supernatants taken at 6 hours and 15 hours post LPS addition were assayed for the production of RANTES, TNFα, IL-1β, IL-6, IL-10, IL-12p40 (subunit common to IL-12 and IL-23) and IL-15 (negative control) using Luminex® technology (Procarta human cytokine kit; Panomics) following the manufacturer's instructions.

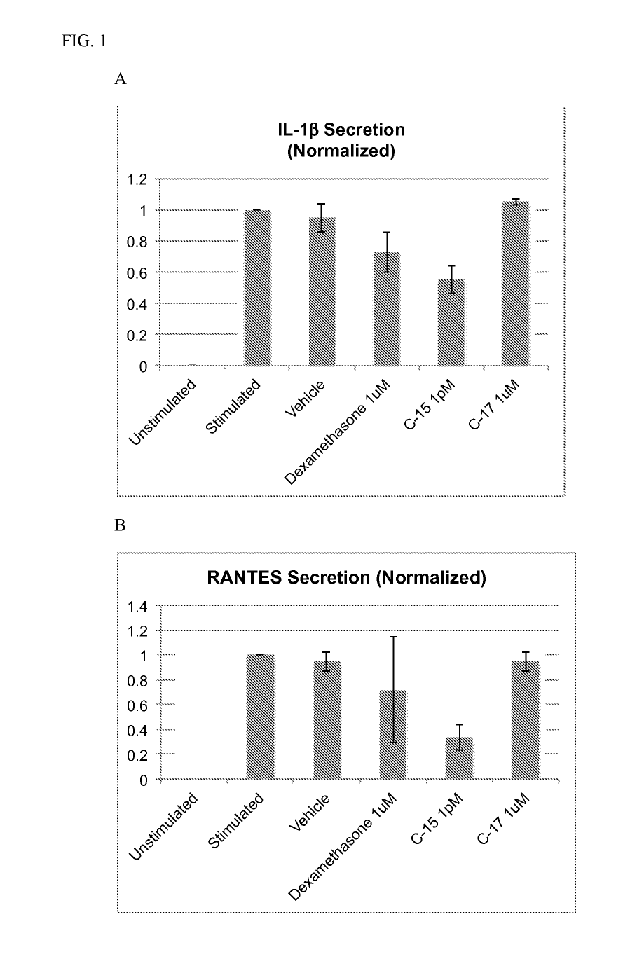

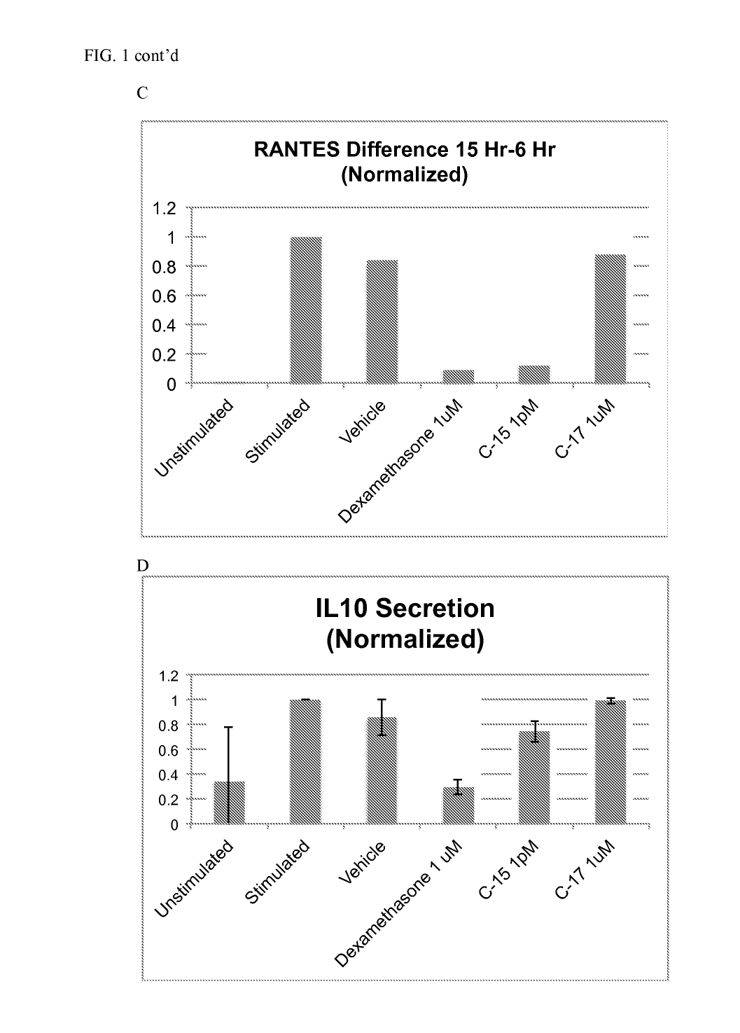

The results for the concentration of IL-1β and RANTES at 16 hours post-stimulation is shown in FIGS. 1A and 1B. FIG. 1C shows the difference in RANTES expression between the 6 hour and 15 hours time points. FIG. 1D shows the IL-10 expression at 16 hours. No inhibition of IL-15 was observed as expected.

At a dose as low as 1 pM, the human chemerin C15 peptide showed strong inhibition of human macrophage secretion of IL-1β and RANTES at 16 hours post-stimulation (approximately 45% and 65%, respectively) (FIGS. 1A and 1B). For newly synthesized RANTES (i.e. the difference between the 6 and 15 hour time points), the inhibition was approximately 90%. The human chemerin C15 peptide also showed strong inhibition of human macrophage secretion of IL-12p40 at 16 hours post-stimulation (approximately 55%) (FIG. 1D). Dexamethasone also exhibited inhibition of IL-1β and RANTES secretion (approximately 30% and 50%, respectively for the 1 μM dosage), but the effect was less than that of C15. Dexamethasone inhibition of IL-12p40 secretion was slightly stronger than that of C15 (FIG. 1D). Dexamethasone also potently inhibited (˜70%) the production of IL-10 which is an anti-inflammatory cytokine, whereas C15 only produced a modest decrease (˜25%) in IL-10 (FIG. 1D). Since IL-10 is naturally anti-inflammatory, it is not desirable to inhibit IL-10. The human chemerin C17 peptide did not exhibit any significant inhibition of cytokine production even at 1 μM. Overall, the human chemerin peptide exhibited superior in potency to dexamethasone by showing similar effect on inflammatory cytokine levels at one millionth the dose.

### Example 2

### Assay for ChemR23 or GPR1 Agonist or Antagonist Activity

Chemerin binds to two G protein-coupled receptors, ChemR23 (CMKLR1), and GPR1 in addition to CCRL2 which is not a G protein-coupled receptor. In order to determine the mode of action of chemerin C15 peptides, the ability of the chemerin peptides to act as antagonists or agonists of GCPRs was examined.

In this experiment, the agonist and/or antagonist activity of human chemerin C15 peptide AGEDPHSFYFPGQFA (SEQ ID NO: 1) was compared to that of a mouse chemerin C15 peptide AGEDPHGYFLPGQFA (SEQ ID NO: 9), human chemerin C16 peptide AGEDPHSFYFPGQFAF (SEQ ID NO: 24) and human chemerin C17 peptide AGEDPHSFYFPGQFAFS (SEQ ID NO: 25).

The DiscoveRx PathHunter™ eXpress GPCR activity assay was employed to test agonist and antagonist activities of the chemerin peptides against the GPCRs ChemR23 and GPR1. Two assay formats were tested, the PathHunter β-Arrestin assay and the Hit Hunter cAMP Hunter assay.

### PathHunter β-Arrestin Assay

The PathHunter β-Arrestin assay monitors the activation of a GPCR in a homogenous, non-imaging assay format using a technology developed by DiscoveRx called complementation, which utilizes an enzyme fragment complementation (EFC) assay with β-galactosidase (β-Gal) as the functional reporter. The enzyme is split into two complementary portions expressed as fusion proteins in the cell. The Enzyme Acceptor (EA) is fused to β-Arrestin and the ProLink donor peptide is fused to the GPCR of interest. Upon GPCR stimulation, β-Arrestin is recruited to the receptor for desensitization, bringing the two fragments of β-Gal together and allowing complementation to occur. This will generate an active enzyme that can convert a chemiluminescent substrate and generate an output signal detectable on a standard microplate reader.

The assay involves CHO cell lines that express 1) a GPCR of interest (e.g. ChemR23 or GPR1) that has a fragment of the βgal enzyme fused to the C-terminus of the receptor and 2) a β-arrestin fused to the main β-gal enzyme. When the agonist binds to the receptor, β-arrestin is recruited to the receptor and the β-gal enzyme is complemented by the fragment from the GPCR thus forming a functional β-gal enzyme. A substrate is then added and luminescence is generated to detect β-arrestin recruitment.

The protocol used was a standard protocol employed by DiscoveRx PathHunter™ profiling service. Briefly, PathHunter cell lines were expanded from freezer stocks in T25 flasks according to standard procedures and maintained in selective growth media prior to assay. Once it was established that the cells were healthy and growing normally, cells were passaged from flasks using cell dissociation reagent and seeded into white walled clear bottom 384-well microplates for compound profiling. For profiling, cells were seeded at a density of 5000 cells per well in a total volume of 20 μL and were allowed to adhere and recover overnight prior to compound addition.

For the agonist assay, intermediate dilution of compound stocks were generated such that 5 μL of 5× compound could be added to each well with a final DMSO concentration of 1% of total volume. For profiling compound in agonist mode, the cells were incubated in the presence of compound at 37° C. for 90 minutes.

For the antagonist assay, agonist dose curves were performed the morning of profiling to determine the EC80 value for the following antagonist testing with compounds. 5 μL of 5× agonist (i.e. chemerin) was added to each well with an equal concentration of vehicle present. EC80 agonist concentration was determined directly from agonist dose curve. For antagonist determination, cells were preincubated with antagonist followed by agonist challenge at the EC80 concentration: 4. 5 μL of 5× compound added to cells and incubated at 37° C. for 30 minutes. 5. 5 μL of 6× EC80 agonist added to cells and incubated at 37° C. for 90 minutes.

Assay signal was generated through a single addition of 12.5 or 15 μL (50% v/v) of PathHunter Detection reagent cocktail for agonist and antagonist assays respectively followed by one hour incubation at room temperature. Microplates were read following signal generation with a PerkinElmer Envision™ instrument for chemiluminescent signal detection

Dose curves in the presence and absence of compound were plotted using GraphPad Prism or Activity Base. For agonist mode assays, percentage activity was calculated using the following formula: % Activity=100%×(Mean RLU of test sample−mean RLU of vehicle control)/(mean MAX RLU control ligand−mean RLU of vehicle control)). For antagonist mode assays, percentage inhibition was calculated using the following formula: % Inhibition=100%×(1−(Mean RLU of test sample−mean RLU of vehicle control)/(mean RLU of EC80 control−mean RLU of vehicle control)).

Hit Hunter cAMP Hunter Assay

DiscoveRx have developed a panel of cell lines stably expressing non-tagged GPCRs that signal through cAMP. The Hit Hunter cAMP Hunter assay monitors the activation of a GPCR via Gi and Gs secondary messenger signaling in a homogenous, non-imaging assay format using a technology developed by DiscoveRx called complementation. This utilizes an enzyme fragment complementation (EFC) assay with βgalactosidase (βGal) as the functional reporter. The enzyme is split into two complementary portions. Pro-Label donor peptide is fused to cAMP and in the assay competes with cAMP generated by cells for binding to a cAMP-specific antibody. Active β-Gal is formed by complementation with EA to any unbound ED-cAMP. The active enzyme can convert a chemiluminescent substrate to generate an output signal detectable on a standard microplate reader.

The protocol used was a standard protocol employed by DiscoveRx PathHunter™ profiling service. Briefly, cAMP Hunter cell lines were expanded from freezer stocks in T25 flasks according to standard procedures and maintained in selective growth media prior to assay. Once it was established that the cells were healthy and growing normally, cells were passaged from flasks using cell dissociation reagent buffer and seeded into white walled clear bottom 384-well microplates for compound profiling. For profiling, cells were seeded at a density of 10000 cells per well in a total volume of 20 μL and were allowed to adhere and recover overnight prior to compound addition. Cells were treated the following day using the protocols shown below. cAMP modulation was determined using the DiscoveRx HitHunter cAMP XS+ assay.

For the agonist assay, media was aspirated from cells and replaced with 15 μL 2:1 HBSS/Hepes:cAMP XS+ Ab reagent. Intermediate dilution of compound stocks were generated such that 5 μL of 4× compound could be added to each well with a final vehicle concentration of 1% of total volume. For profiling compound in agonist mode, the cells were incubated in the presence of compound at 37° C. for 30 minutes.

For the antagonist assay, media was aspirated from cells and replaced with 10 μL 1:1 HBSS/Hepes:cAMP XS+ Ab reagent. Agonist dose curves were performed to determine the EC80 value for the following antagonist testing with compounds. 5 μL of 4× agonist (i.e. chemerin) was added to each well with an equal concentration of vehicle present. EC80 agonist concentration was determined directly from agonist dose curve. For antagonist determination, cells were pre-incubated with antagonist followed by agonist challenge at the EC80 concentration. 5 of 4× compound was added to cells and incubated at 37° C. for 30 minutes. 5 μL of 4× EC80 agonist was added to cells and incubated at 37° C. for 30 minutes.

Assay signal was generated through incubation with 20 μL cAMP XS+ ED/CL lysis cocktail for one hour followed by incubation with 20 μL cAMP XS+ EA reagent for three hours at room temperature. Microplates were read following signal generation with a PerkinElmer Envision™ instrument for chemiluminescent signal detection.

Dose curves in the presence and absence of compound were plotted using GraphPad Prism or Activity Base. For agonist mode assays, percentage activity is calculated using the following formula: % Activity=100%×(mean RLU of test sample−mean RLU of vehicle control)/(mean RLU of MAX control−mean RLU of vehicle control). For antagonist mode assays, percentage inhibition is calculated using the following formula: % Inhibition=100%×(1−(mean RLU of test sample−mean RLU of vehicle control)/(mean RLU of EC80 control−mean RLU of vehicle control)).

A summary of the data is provided in Table 1 below for the GPR1 and CMKLR1 PathHunter Biosensor cell lines.

TABLE 1
GPR1 and CMKLR1 PathHunter Biosensor Data

| | Compound | [EC50] | % Max | Rank | [IC50] | % Max |
|---|---|---|---|---|---|---|
| GPCR | ID | (M) | Activity | Order | (M) | Inhibition |
| GPR1 | mC15 | 3.7E−06 | 27.8% | 4 | >1.0E−5 | 0% |
| | C15 (human) | 1.7E−02 | 10.6% | 3 | >1.0E−5 | 0% |
| | C16 (human) | 2.1E−09 | 87.9% | 2 | >1.0E−5 | 0% |
| | C17 (human) | 1.5E−09 | 80.9% | 1 | >1.0E−5 | 0% |
| ChemR23 | mC15 | >1.0E−5 | 0.4% | 3 | >1.0E−5 | 0% |
| | C15 (human) | >1.0E−5 | 0.8% | 3 | >1.0E−5 | 0% |
| | C16 (human) | 2.9E−08 | 98.6% | 1 | >1.0E−5 | 0% |
| | C17 (human) | 4.8E−07 | 67.5% | 2 | >1.0E−5 | 0% |

A summary of the data is provided in Table 2 below for the mouse ChemR23 PathHunter and human ChemR23 cAMP Hunter Biosensor cell lines. PathHunter Biosensor cell lines.

TABLE 2
ChemR23 PathHunter and human ChemR23 cAMP Hunter Biosensor Data

| Compound Name | AssayName | AssayFormat | AssayTarget | ResultType | RC50 (uM) |
|---|---|---|---|---|---|
| hrChemerin | Arrestin | Agonist | mChemR23 | EC50 | 0.0015405 |
| hrChemerin | cAMP | Agonist | ChemR23 | EC50 | 0.0040557 |
| mC15 | Arrestin | Agonist | ChemR23 | EC50 | >10 |
| mC15 | Arrestin | Antagonist | m ChemR23 | IC50 | >10 |
| mC15 | cAMP | Antagonist | ChemR23 | IC50 | >10 |
| C15 (human) | Arrestin | Agonist | m ChemR23 | EC50 | >10 |
| C15 (human) | Arrestin | Antagonist | m ChemR23 | IC50 | 9.6635 |
| C15 (human) | cAMP | Antagonist | ChemR23 | IC50 | >10 |
| C16 (human) | Arrestin | Agonist | m ChemR23 | EC50 | 0.038472 |
| C16 (human) | Arrestin | Antagonist | m ChemR23 | IC50 | >10 |
| C16 (human) | cAMP | Antagonist | ChemR23 | IC50 | >10 |
| C17 (human) | Arrestin | Agonist | m ChemR23 | EC50 | 0.84015 |
| C17 (human) | Arrestin | Antagonist | m ChemR23 | IC50 | >10 |
| C17 (human) | cAMP | Antagonist | ChemR23 | IC50 | >10 |

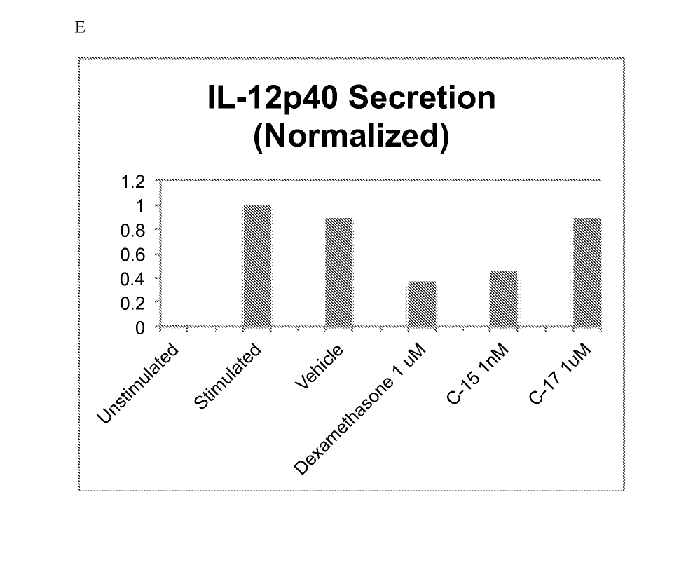

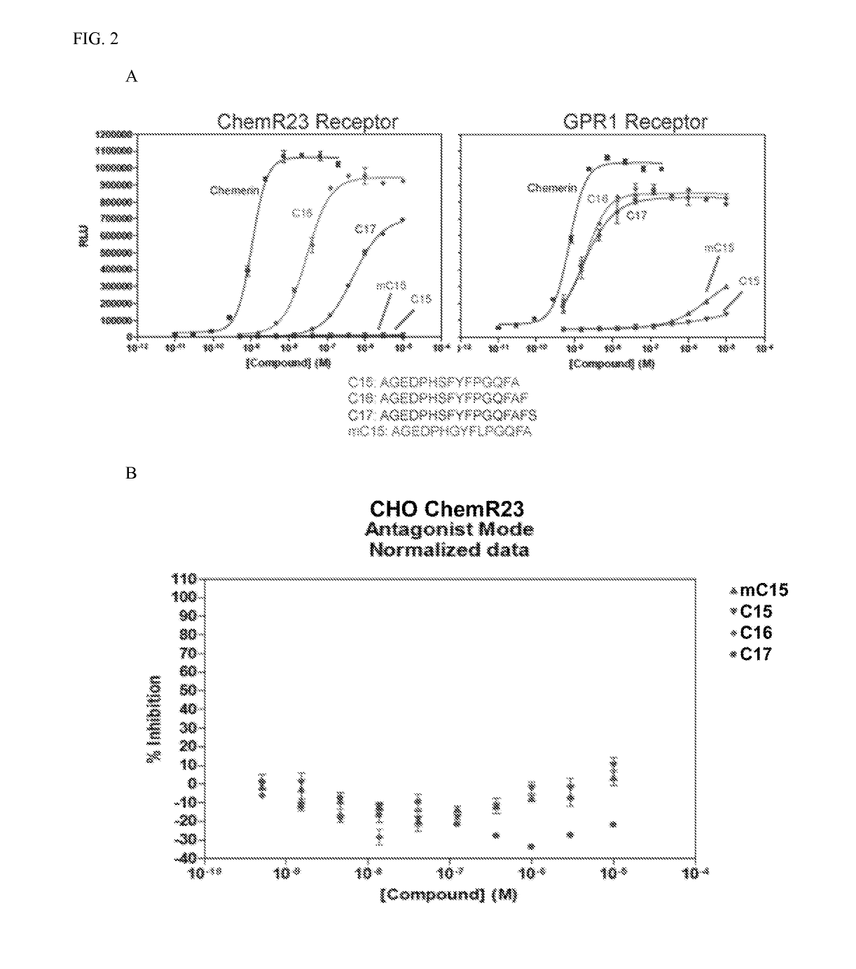

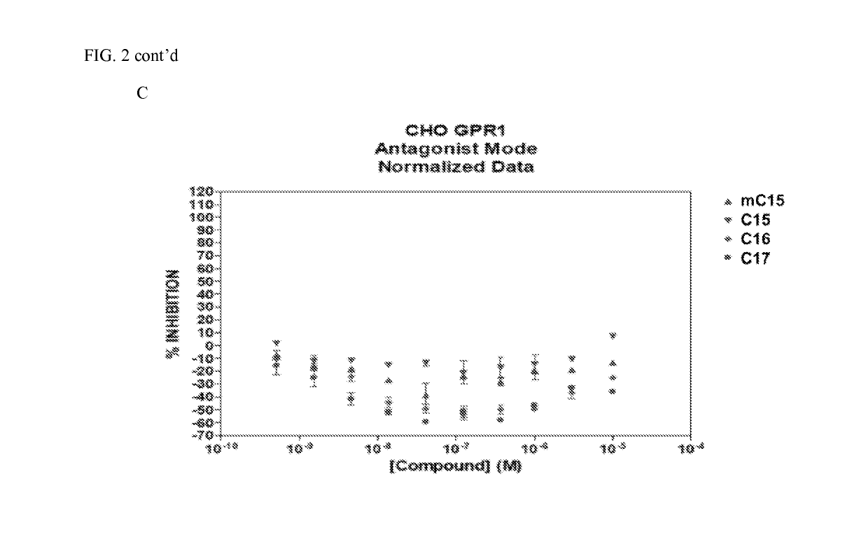

Agonist dose response curves for ChemR23 and GPR1 receptors are shown in FIGS. 2A and 2B. As shown in the table above and in the figure, neither human nor mouse chemerin C15 peptides acted as agonists for human ChemR23 or GPR1. Chemerin exhibited potent agonist activity for both receptors as expected. In addition, both human chemerin C16 and C17 peptides exhibited agonist activity.

For the antagonist assays, chemerin was stimulated to 80% maximum signal and antagonized with the chemerin peptides. Antagonist dose response curves for ChemR23 and GPR1 receptors are shown in FIGS. 2C and 2D. As shown in the table above and in the figure, neither human nor mouse chemerin C15 peptides acted as antagonists for human ChemR23 or GPR1.

### Example 3

### Effect of Alanine Substitution in FYFP Motif (SEQ ID NO: 2) on C15 Anti-Inflammatory Activity

The B-subunit of protein phosphatase 2A contains a FYFP motif (SEQ ID NO: 2) that is similar to the FYFP motif (SEQ ID NO: 2) in the human chemerin C15 peptide. This FYFP motif (SEQ ID NO: 2) is conserved across species and is critical for binding to the PP2A core enzyme (Davis A J, et al. J Biol Chem. 2008; 283:16104-14). The human wild-type PP2A B-subunit PR70 comprises the amino acid sequence IPTFYFPRGRP (SEQ ID NO: 26).

In this experiment, the importance of the FYFP motif (SEQ ID NO: 2) in the human chemerin C15 peptide on anti-inflammatory activity was examined. The ability of the human chemerin C15 peptide AGEDPHSFYFPGQFA (SEQ ID NO: 1) was compared to that of a substituted chemerin C15 peptide having the amino acid sequence AGEDPHGYFAPGQFA (SEQ ID NO: 27), where the second phenylalanine in the peptide is modified to alanine. The experiment was performed as described in Example 1. 0.1 pM 0.5 pM and 1 pM concentrations of the C15 and C15 mutant peptides were tested. Cytokine expression was determined as described in Example 1.

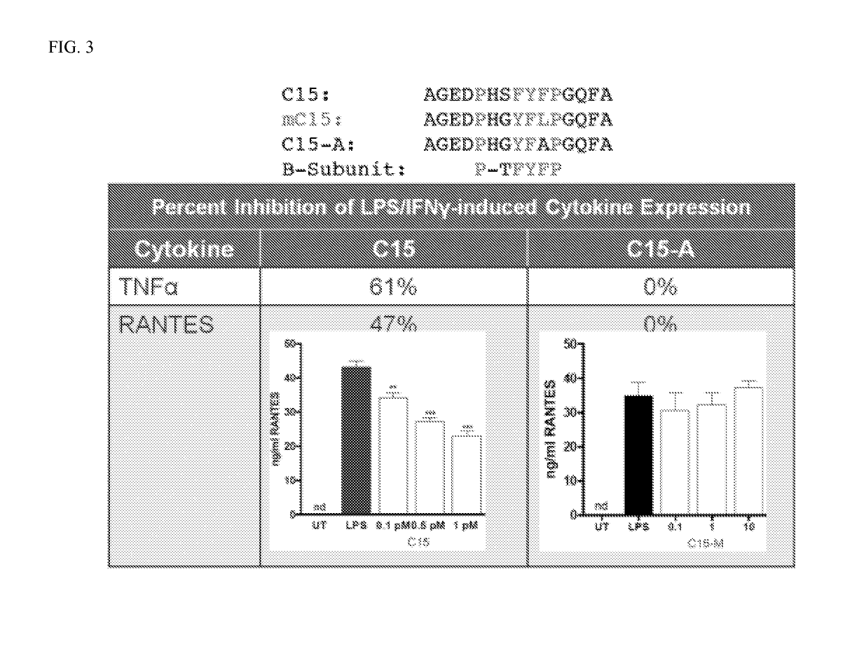

FIG. 3 shows the percent inhibition of TNFα and RANTES expression in the presence of the C15 or C15 alanine substituted peptides. As shown in the figure, the C15 peptide was able to inhibit TNFα and RANTES expression by 61% and 47% respectively. In contrast, the mutant C15 polypeptide was unable to inhibit expression of either cytokine. This data demonstrates that the FYFP motif (SEQ ID NO: 2) is important for the anti-inflammatory properties of the chemerin C15 peptide.

### Example 4

### Ointment Formulation of Human Chemerin C15 Peptide

In this example, Human chemerin C15 peptide was formulated as an ointment follows:

TABLE 3

| Component | Amount |
|---|---|
| Human chemerin C15 | 2.6 +/− 0.8 mg/g ointment |
| peptide | |
| White Petroleum | 50% |
| Caprylic Capric | 45% |
| Triglyceride | |
| Beeswax | 5% |

In additional examples of an ointment, human chemerin C15 peptide is formulated as follows:

TABLE 4

| Component (% w/w) | Ointment 2728-74 | Ointment 2728-75 |
|---|---|---|
| Human chemerin C15 | 2.6 +/− 0.8 mg/g | 2.6 +/− 0.8 mg/g |
| peptide | ointment | ointment |
| Dimethyl isosorbide | — | 10% |
| Butylated hydroxytoluene | 0.02% | 0.02% |
| PEG 400 | 15% | — |
| Span 80 | 2% | 2% |
| White wax | 10% | 10% |
| White petrolatum | 71.98% | 76.98$ |

### Example 5

### Gel Formulation of Human Chemerin C15 Peptide

In this example, human chemerin C15 peptide is formulated as an gel follows:

TABLE 5

| | | |
|---|---|---|
| Component (% w/w) | Gel 2728-60 | Gel 2728-76 |

| | | |
|---|---|---|
| Human chemerin C15 | 2.6 +/− 0.8 mg/ml gel | 2.6 +/− 0.8 mg/ml gel |
| peptide | | |
| Dimethyl isosorbide | 15% | 15% |
| Transcutol | 25% | 25% |
| Hexylene glycol | 12% | 12% |
| Propylene glycol | 5% | 5% |
| Methylparaben | 0.15% | 0.15% |
| Propylparaben | 0.05% | 0.05% |
| EDTA | 0.01% | 0.01% |
| Hydroxyethyl cellulose | — | 1% |
| Penmulen TR-1 | 0.5% | — |
| 25% Trolamine | q.s. pH 6.0 | q.s. pH 4.5 |
| Water | q.s. 100% | q.s. 100% |

### Example 6

### Lotion Formulation of Human Chemerin C15 Peptide

In this example, human chemerin C15 peptide is formulated as an lotion follows:

TABLE 6

| Component (% w/w) | Lotion 2728-77 | Lotion 2728-72 |
|---|---|---|
| Human chemerin C15 | 2.6 +/− 0.8 mg/ml | 2.6 +/− 0.8 mg/ml |
| peptide | lotion | lotion |
| Dimethyl isosorbide | 13% | 13% |
| Transcutol | 20% | 20% |
| Hexylene glycol | 10% | 10% |
| Propylene glycol | 4% | 4% |
| Methylparaben | 0.15% | 0.15% |
| Propylparaben | 0.05% | 0.05% |
| EDTA | 0.01% | 0.01% |
| Carbopol Ultrez 10 | 0.5% | 0.3% |
| Penmulen TR-1 | 0.2% | 0.2% |
| Isopropyl myristate | 3% | — |
| Oleyl alcohol | 5% | — |
| Cetyl alcohol | — | 2% |
| Light mineral oil | — | 5.5% |
| Oleic acid | — | 5% |
| Butylated hydroxytoluene | 0.2% | 0.2% |
| White petrolatum | 5% | — |
| 25% Trolamine | q.s. pH 6.0 | q.s. pH 6.0 |
| Water | q.s. 100% | q.s. 100% |

### Example 7

### Solution Formulation of Human Chemerin C15 Peptide

In this example, human chemerin C15 peptide is formulated as a solution follows:

TABLE 7

| | Solution | Solution | Solution | |
|---|---|---|---|---|
| Component (% w/w) | 2728-79 | 2728-81 | 2728-80 | Solution A |
| Human chemerin | 2.6 +/− 0.8 | 2.6 +/− 0.8 | 2.6 +/− 0.8 | 2.6 +/− 0.8 |
| C15 peptide | mg/ml | mg/ml | mg/ml | mg/ml |
| | solution | solution | solution | solution |
| Dimethyl | 15% | 15% | — | |
| isosorbide | | | | |
| Transcutol | 25% | 25% | — | |
| Hexylene glycol | 12% | 12% | — | |
| Propylene glycol | 5% | 5% | — | |
| DMSO | — | — | 99% | |
| 25% Trolamine | q.s. pH 4.5 | q.s. pH 6.0 | — | |
| Isopropyl | | | | 45% |
| myristate | | | | |
| Alcohol | | | | 45% |
| Undecylenic acid | | | | 5% |
| Sodium lauryl | | | | 5% |
| sulfate | | | | |
| Water | q.s. 100% | q.s. 100% | — | |

### Example 8

### Skin Stability and Penetration of Human Chemerin C15 Peptide

In this example, the ability of the human C15 peptide to remain stable in and to penetrate human skin was examined. A DMSO form and an ointment comprising the C15 peptide were tested.

### Chemerin C15 Peptide Ointment

The objective of the study was to determine whether human chemerin C15 peptide would diffuse through in vitro human skin maintained under flow-through conditions in Franz cells where the C15 peptide is administered as an ointment. Human chemerin C15 peptide was prepared as an ointment as described in Example 4. A 10% solution of the C15 ointment was prepare immediately prior to skin application. Female human skin obtained from abdominoplasty was maintained in tissue media and antibiotics and used within 3 days.

A standard Franz diffusion cell (LGA, Berkeley, Calif.) was used under static conditions (n=3). Approximately 200 μl of the 10% ointment solution was transferred to the surface of the skin and distributed on the surface by spatula. A thin liner was then applied for light pressure to the skin surface for 5 min after which the diffusion cell was occluded and maintained for 24 hours. After this time, the ointment was recovered by scraping a spatula over the skin surface and transferring the retained material to a 50/50 water-chloroform solution. The epidermis and dermis were then separated by heat and the epidermis extracted with a 50/50 water-chloroform solution. The epidermis was then transferred to a second tube and homogenized in PBS containing 0.1% protease inhibitor. The dermis was minced and homogenized in PBS containing 0.1% protease inhibitor. The receptor fluid was recovered and concentrated under vacuum. Ointment without C15 was applied to skin and the skin sampled in the same manner as a control (n=2).

C15 recovery from the dosing material, epidermis, and receptor fluid was determined by HPLC. C15 concentration in the dermis was determine by LC/MS. The skin surface and epidermis recoveries and epidermis homogenate samples were analyzed using the following reversed phase HPLC conditions:

TABLE 8

| | |
|---|---|
| HPLC | Shimadzu 20A system |
| Mobile phase | A-0.1% formic acid in water |
| | B-0.1% formic acid in acetonitrile |
| Column | Phenomenex Gemini ™ C18 column (Cat. No. |
| | 00B-4439-E0, 4.6 × 50 mm, 3 μm) |
| Injection Volume | 5 μl |
| Gradient | 80% A + 20% B to 10% A + 90% B (0-3 min) |
| | and 10% A + 90% B (3-3.5 min) |
| Flow rate | 800 μl/min |
| Detection | peak height at 275 nm at 1.92 min |
| LLQ | 150 ng/ml |

The dermis samples were analyzed using the following LC/MS/MS conditions:

TABLE 9

| | |
|---|---|
| HPLC | Shimadzu VP system with Shimadzu SIL-HTc |
| | autosampler |
| Mobil phase | A-0.2% formic acid in water |
| | B-0.2% formic acid in acetonitrile |
| Column | 2.1 × 10 mm Peeke Scientific Duragel G C18 |
| | guard cartridge |
| Injection Volume | 100 μl |
| Gradient | 5% B (0.5 min) then 5-95% B (2 min) |
| Flow rate | 400 μl/min |
| Mass Spectrometer | Applied Biosystems/MDS SCIEC API 3000 |
| Interface | TurboIonSpray (ESI) at 400° C. |
| Software | Analyst v1.4.1 |
| Polarity | Positive Ion |
| Q1/Q3 Ions | 803.7/120.4 for C15 |
| | 256.2/167.2 for diphenyhydramine (I.S.) |
| | 272.1/215.2 for dextromethorphan (I.S.) |
| LLQ | 10 ng/ml |

Good mass balance was achieved with the sample recovery and extraction methods. Chloroform may have removed some C15 that had initially penetrated the epidermis. Low amounts of C15 were measured in the epidermis and dermis. Combined, both compartment accounted for less the 1% of the applied dose.

TABLE 10

| C15 | Skin | | | | Dermis | | | |
|---|---|---|---|---|---|---|---|---|
| applied | Surface | | Epidermis | | homogenate | | Receptor | Total |
| mg | mg | % | mg | % | ng | % | fluid | % |
| 2.19 | 0.74 | 33.6 | 1.53 | 70.2 | 0 | 0.00 | <LLQ | 103.8 |
| 3.52 | 1.17 | 33.3 | 2.25 | 64.1 | 77.4 | 0.02 | <LLQ | 97.4 |
| 2.06 | 0.83 | 40.6 | 1.45 | 71.2 | 238.2 | 0.12 | <LLQ | 111.8 |

### 50% DMSO Solution Study

The objective of the study was to determine whether human C15 peptide would diffuse through in vitro human skin maintained under flow-through conditions in Franz cells with 50% DMSO in water. 50% DMSO is considered an acceptable maximum for penetration enhancement.

The samples used in the study were mouse and human chemerin C15 peptides stored at −20 C°. The skin sample used was female human skin obtained from mammoplasty. A frozen sample was stored at −20 C.° for 30 days. A fresh sample was obtained in tissue media and antibiotics and used within 3 days.

### Stability Study:

An initial study was performed comparing stability of human C15 versus Mouse C15. Homogenates of frozen and fresh human skin were prepared to evaluate the degradation of C15 in skin. Frozen or fresh human skin were separately minced and homogenized in 3 ml water and the supernatant isolated. Supernatant was mixed with solutions of mouse or human C15 to yield a 0.5 mg/ml C15 solution. Each solution was incubated at 37° C. and samples were taken at 0, 1, 2 and 24 hours for analysis of C15 (FIG. 3).

Human C15 was more stable than mouse C15 in this assay. Degradation of C15 was substantially lower in homogenates of frozen than of fresh skin. After 24 hours, C15 degradation in homogenate from frozen and fresh skin was 25% and 98%, respectively. Based on these findings, a 2% solution of human C15 was prepared for the diffusion cell tests.

### Franz Cell Studies:

Two studies of the dermal penetration of C15 were conducted with Franz cells:

1. A 1% solution of mouse C15 in 50% DMSO in water was applied to previously frozen human skin to develop the HPLC method for subsequent tests with human C15. This was done in triplicate.
2. A 2% solution of human C15 in 50% DMSO in water was applied to fresh human skin and epidermis, dermis, and receptor fluid were analyzed for C15.

Skin was rinsed, blotted dry, cut into circular pieces and conditioned in the Franz cell for 2 hours prior to C15 application. Flow-through, water-jacketed diffusion cells that exposed a skin area of 2.54 cm2 were used. The cells were maintained at 37° C., operated under static conditions and stirred at 700 rpm for 24 hr. PBS (pH=7.0) was used as the receptor fluid. C15 solutions in 50% DMSO in water was prepared on the day of the experiments.

400 μl of each C15 solution was pipetted in aliquots of 100 μl onto the skin surface and the diffusion cell sealed with parafilm. Diffusion cells were run in triplicate with a single control consisting of skin treated with vehicle only. Receptor fluid (≈5 mL) was collected at the end of 24 hours and concentrated by evaporation prior to analysis. The skin was blotted dry, tape-stripped three times to remove residual C15 and heat-separated at 50° C. into epidermis and dermis. Epidermis was sonicated in 5% TCA for 10 minutes and the supernatant analyzed. Dermis was minced and homogenized in 5% TCA and the supernatant concentrated and analyzed.

For the mouse C15 experiment only the receptor fluid was analyzed.

A reverse-phase HPLC method was developed to quantify Human C15 (Shimamura et al., 2009). The separation was achieved using a Phenomenex Gemini™ C18 column (Cat. No. 00B-4439-E0, 4.6×50 mm, 3 μm) at 40° C. in the Shimadzu 20A system. The mobile phase was mixed with (A) 0.1% formic in water and (B) 0.1% formic acid in acetonitrile. The separation was conducted using a gradient system of 80% A+20% B to 10% A+90% B (0-3 min) and 10% A+90% B (3-3.5 min) at a flow rate of 0.8 ml/min. The injection volume was 5 μl. The eluent was monitored at 275 nm Human C15 was observed as a single peak in the chromatogram with retention time at about 1.8 min. The quantification of Human C15 was achieved by external standard calibration. Results for human C15 are presented as % absorbed of the applied dose. See Table 11.

Very low levels of C15 were measured in the receptor fluid from each study. C15 receptor fluid levels were highest using frozen human skin and mouse C15 (0.3%). Human C15 was detected in the receptor fluid and epidermis. A broad peak at 1.8 min was observed with the dermis samples but could not be distinguished from a background peak. (Table 11). HPLC results and % absorbed for human C15 in fresh human skin (n=3).

TABLE 11

| | | | Total C15 | % C15 |
|---|---|---|---|---|
| | | | passaged | in skin |
| | Peak area | | through | compart- |
| Sample | 1.7-1.8 min | Net C15 (ug) | skin (ug) | ment |
| Skin 1 receptor fluid | 255 | 0.0053 | 1.26 | 0.02% |
| Skin 2 receptor fluid | 1587 | 0.0332 | 7.34 | 0.09% |
| Skin 3 receptor fluid | 84 ND | 0.0018 | 0.42 | 0.01% |
| Control receptor fluid | | | | |
| Skin 1 epidermis | 165899 | 3.50 | 700.72 | 8.76% |
| Skin 2 epidermis | 139517 | 2.95 | 590.32 | 7.38% |
| Skin 3 epidermis | 49493 | 1.07 | 213.58 | 2.67% |
| Control epidermis | ND | | | |
| Skin 1 dermis | Broad peak | | | |
| Skin 2 dermis | Broad peak | | | |
| Skin 3 dermis | Broad peak | | | |
| Control dermis | Broad peak | | | |

Human C15 does penetrate through human skin under in vitro flow-through conditions using penetration enhancement of 50% DMSO in water. Low levels are detected in the receptor fluid however higher levels are detected in the epidermis and most likely in the dermis.

The results from the two Frnz cell studies described above are summarized in the table below. The studies demonstrated that therapeutically relevant levels of C15 (e.g., >1 nM) can be delivered across the stratum corneum to the dermis or beyond. Penetration enhancers (e.g. DMSO) may not be necessary to achieve delivery to the dermis.

TABLE 12

| | DMSO (50%) | | Ointment | |
|---|---|---|---|---|
| Sample | [C15] | | [C15] | |
| Skin1 Epidermis (2.54 cm²) | 419,400 | nM | 953,000 | nM |
| Skin2 Epidermis (2.54 cm²) | 353,500 | nM | 1,406,000 | nM |
| Skin3 Epidermis (2.54 cm²) | 127,000 | nM | 906,000 | nM |
| Skin1 Dermis (2.54 cm²) | NA* | | 48 | nM |
| Skin2 Dermis (2.54 cm²) | NA* | | 149 | nM |
| Skin3 Dermis (2.54 cm²) | NA* | | NS* | |
| Skin1 Receptor Fluid (5 mL) | 151 | nM | <10 | nM |
| Skin2 Receptor Fluid (5 mL) | 888 | nM | <10 | nM |
| Skin3 Receptor Fluid (5 mL) | 50 | nM | <10 | nM |

*NA no analysis possible, interference with HPLC detection.
*NS no sample
Study Outline: Formulated huC15 in DMSO (50% in water) or Ointment (50% Petrolatum, 45% coconut oil, 5% beeswax, no penetration enhancer) applied to fresh human skin. Epidermis, dermis and receptor fluid analyzed by HPLC or LCMS/MS (Ointment) for C15 after 24 hrs.

### Example 9

### Microplaque Assay in Psoriasis patients

The microplaque assay has been used successfully in evaluating topical treatments for psoriasis. The microplaque assay enables the direct comparison of different topical treatments and dosing's directly on psoriatic lesions. A template with 6 holes is adhered to a lesion. Patients visit the clinic daily to have a specific drug dose applied to a metal disk, each disk is then applied to a specific spot, and the arm is then wrapped and kept under occlusion until the next dosing occurs. Multiple formulations, control, and if desired, active comparator, can all be accommodated on one plaque. A typical microplaque assay involves 12-15 patients for 2 weeks.

In order to establish the clinical efficacy and bioavailability of C15 as a topical treatment for psoriasis, a Phase 0 microdosing study of two prototypical topical formulations of C15 is performed in patients with stable plaque psoriasis. In an exemplary microdosing study, a microplaque assay is performed wherein formulated drug is applied daily for 10 to 21 days to one of six test spots (2 cm diameter) on a single stable plaque on each of 15 test subjects. This format allows for the testing of C15 at 2 formulations and 3 concentrations with controls for each formulation and a medium strength steroid (Dexamethasone) or betamethasone Valorate as an active comparator.

In the study, all patients in each cohort will receive daily 0.2 ml applications of each test article applied to one of six uniform test sites cut into a hydrocolloid dressing which is placed over the study plaque on each patient. The test articles are applied by an investigator in a clinical setting during clinic hours. After application of each dose, the study plaque is occluded with an additional dressing until the next clinic visit. Delivering drug in excess and under occlusion greatly enhances the performance of the formulation and drug efficacy relative to more typical Phase 2/3 study designs in psoriasis. Even drugs such as Vitamin D analogs with slow onset (4-6 weeks in self-dosing patients) and very modest efficacy have demonstrated measurable improvement in the microplaque assay. Subjects are seen in the clinic for assessment of condition following treatment. The hydrocolloid dressing is removed, a digital image of the treated plaque is obtained, the treated sites is clinically scored, physical examination is performed, and samples for safety labs are collected. Total Clinical Score (TCS) of each treatment site is recorded at baseline, at pre-determined time period(s) during the study and following the last dosing. The TCS is the sum of erythema (0-3), scaling (0-3) and thickness (0-3). For each sign: 0=none; 1=mild; 2=moderate; 3=severe. The possible range for TCS is 0 to 9. In addition a Dynamic Severity Score (DSS) comparing each site to adjacent untreated area of the psoriasis plaque is recorded at baseline, at pre-determined time period(s) during the study and following the last dosing. The DDS is a 5-point system: −1=worsened; 0=unchanged; 1=slight improvement; 2=clear improvement but not completely clear; 3=completely cleared. Efficacy measures of TCS and DSS are evaluated using descriptive statistics including mean, standard deviations, median, minimum, maximum, and percent change from baseline. All adverse events, including local and systemic events, reported during the study are listed, documenting course, severity, and outcome. All non-solicited adverse events are summarized by treatment group, severity, and relationship to study drug.

Additional microdosing studies can be designed to provide further exploration of additional formulations for informing Phase 2 studies or can be extended in length to address modest activity or slow onset of efficacy.

C15 in vitro inhibits cytokine production/secretion 40-60% within 15 hours. C15 appears to also inhibit cytokine message production. A recent study of the levels of IL-23 in involved and uninvolved skin from psoriasis patients demonstrates that IL-23 levels are 2-fold higher in the plaque than in non-involved skin. Our expectation that the onset of C15 effect will be observable in a microplaque time course is based on results obtained in a Phase 1 study of Stelara in which psoriasis patients showed a 50% improvement in PASI score within two weeks after a single dose. This same cohort of patients achieved maximal serum concentrations at 5 days post-injection. Stelara appears to achieve its therapeutic effect by clearing IL-23 via antibody-antigen binding and by inhibiting IL23p19 message.

### Example 10

### Activity of C15 in a Mouse Model of Psoriasis

In this example, the therapeutic activity of a human chemerin C15 peptide is tested in a mouse model of psoriasis. K5.Stat3C recombinant mice resemble human psoriasis based on clinical, histological, immunophenotypic, and biochemical criteria used to evaluate animal models of psoriasis. The K5.Stat3C mice constitutively express activated Stat2 in keritinocytes and epidermal hyperplasia upon stimulation with 12-0-tetradecanoylphorbol-13-acetate (TPA) topical treatment.

In an exemplary protocol, mice are treated topically on the ear with TPA (e.g. 3.4 nmol TPA in acetone) or acetone control to induce skin lesions 3 times per week for 4-8 weeks. Real-time PCR in skin samples is used to confirm upregulation of cytokine expression, including IL-23, IL012, TNF-α, IL-β, and/or IL-6. Following induction of skin lesions, formulations comprising the human chemerin C15 peptide or vehicle control are applied topically to the skin lesions daily for 6-12 days. Improvement in the lesions is assessed daily. It is expected that mice treated with the formulations containing the human chemerin C15 peptide will exhibit decreased cytokine expression in the psoriatic lesions and improvement in the psoriatic phenotype of the epidermis as assessed by visual inspection and histological examination of skin samples from the treated versus untreated mice.

### Example 11

### Contact Hypersensitivity Assay

In this example, the therapeutic activity of a human chemerin C15 peptide is tested in a contact hypersensitivity assay, which is an in vivo assay of cell-mediated immune function and a model for human allergic contact dermatitis. In this assay, epidermal cells are exposed to exogenous haptens which results in a delayed-type hypersensitive reaction that can be measured and quantified. The Langerhans cell, which is an Ia⁺, bone marrow-derived, epidermal cell, initiates sensitization to haptens by presenting antigens to CD4-bearing T lymphocytes, which, in turn, secrete lymphokines and recruit other cells to the site of the reaction.

Contact hypersensitivity consists of the afferent or initial sensitizing phase, and the efferent or elicitation phase. During the efferent phase, when epidermal cells encounter a particular antigen to which they have previously been exposed, localized swelling occurs (in rodents) and in humans results in eczema of the skin.

In an exemplary protocol, mice are shaved and the skin of their abdomens exposed to a hapten. After 6 days (the afferent phase), the baseline ear thickness is measured prior to initiation of the efferent phase. Finally, the ear is treated epicutaneously with the hapten solution and ear thickness is measured at approximately 24 hr. The model contact allergen used in the study is 2,4,6-trinitrochlorobenzene (TNCB; also known as picryl chloride) dissolved in an acetone/olive oil solution. Other exemplary allergens that can be used include, for examples, FITC, oxazalone, and DNFB. The change in ear thickness after allergen treatment can be used to calculate the percent suppression of contact hypersensitivity. In exemplary embodiments, the mice are pre-treated with a formulation comprising a human chemerin C15 peptide to examine prevention or suppression of the allergic response. In additional exemplary embodiments, the mice are co-administer the hapten with a formulation comprising a human chemerin C15 peptide to examine prevention or suppression of the allergic response. In additional exemplary embodiments, the mice are treated with the hapten to induce the allergic response and then treated with a formulation comprising a human chemerin C15 peptide to examine treatment of the allergic response. It is expected that treatment with the human chemerin C15 peptide will result in prevention, suppression and/or treatment of the allergic response.

The examples and embodiments described herein are for illustrative purposes and various modifications or changes suggested to persons skilled in the art are to be included within the spirit and purview of this application and scope of the appended claims. The section headings used herein are for organizational purposes only and are not to be construed as limiting the subject matter described.

1. A topical formulation comprising:
(a) a chemerin C15 peptide in an amount effective for the treatment of an inflammatory dermatological disorder; and
(b) a pharmaceutically acceptable excipient for topical administration;
wherein the formulation minimizes systemic exposure.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. The topical formulation of claim 1, wherein the chemerin C15 peptide is a human chemerin C15 peptide.

17. The topical formulation of claim 16, wherein the human chemerin C15 peptide comprises the sequence of amino acids AGEDPHSFYFPGQFA.

18. (canceled)

19. (canceled)

20. (canceled)

21. (canceled)

22. (canceled)

23. (canceled)

24. (canceled)

25. The topical formulation of claim 1, wherein the formulation comprises at least one of: petrolatum, caprylic triglyceride and beeswax.

26. The topical formulation of claim 25, wherein the ointment comprises about 50% petrolatum, about 45% caprylic triglyceride and about 5% beeswax.

27. The topical formulation of claim 1, wherein the formulation comprises butylated hydroxytoluene, PEG 400, Span 80, white wax, and white petrolatum.

28. The topical formulation of claim 27, wherein the formulation comprises about 0.02% w/w butylated hydroxytoluene, about 15% w/w PEG 400, about 2% w/w Span 80, about 10% w/w white wax, and about 71.98% w/w white petrolatum.

29. The topical formulation of claim 1, wherein the formulation comprises butylated dimethyl isosorbide, butylated hydroxytoluene, Span 80, white wax, and white petrolatum.

30. The topical formulation of claim 29, wherein the formulation comprises about 10% w/w dimethyl isosorbide, about 0.02% w/w butylated hydroxytoluene, about 2% w/w Span 80, about 10% w/w white wax, and about 76.98% w/w white petrolatum.

31. (canceled)

32. (canceled)

33. (canceled)

34. The topical formulation of claim 1, wherein the formulation comprises isopropyl myristate, alcohol, undecylenic acid and sodium lauryl sulfate.

35. The topical formulation of claim 34, wherein the formulation comprises about 45% isopropyl myristate, about 45% alcohol, about 5% undecylenic acid and about 5% sodium lauryl sulfate.

36. (canceled)

37. The topical formulation of claim 1, wherein the formulation comprises about 50% DMSO, and about 50% water.

38. The topical formulation of claim 1, wherein the formulation comprises dimethyl isosorbide, Transcutol, hexylene glycol, and propylene glycol.

39. The topical formulation of claim 38, wherein the formulation comprises about 15% w/w dimethyl isosorbide, about 25% w/w Transcutol, about 12% w/w hexylene glycol, and about 5% w/w propylene glycol.

40. (canceled)

41. (canceled)

42. (canceled)

43. (canceled)

44. The topical formulation of claim 1, wherein the formulation comprises Dimethyl isosorbide, Transcutol, Hexylene glycol, Propylene glycol, Methylparaben, Propylparaben, EDTA, Carbopol Ultrez 10, Penmulen TR-1, and Butylated hydroxytoluene.

45. The topical formulation of claim 1, wherein the formulation comprises Dimethyl isosorbide, Transcutol, Hexylene glycol, Propylene glycol, Methylparaben, Propylparaben, EDTA, Carbopol Ultrez 10, Penmulen TR-1, Isopropyl myristate, Oleyl alcohol, Butylated hydroxytoluene, and White petrolatum.

46. The topical formulation of claim 45, wherein the formulation comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.5% w/w Carbopol Ultrez 10, about 0.2% w/w Penmulen TR-1, about 3% w/w Isopropyl myristate, about 5% w/w Oleyl alcohol, about 0.2% w/w Butylated hydroxytoluene, and about 5% w/w White petrolatum.

47. The topical formulation of claim 1, wherein the formulation comprises Dimethyl isosorbide, Transcutol, Hexylene glycol, Propylene glycol, Methylparaben, Propylparaben, EDTA, Carbopol Ultrez 10, Penmulen TR-1, Cetyl alcohol, Light mineral oil, Oleic acid, Butylated hydroxytoluene.

48. The topical formulation of claim 47, wherein the formulation comprises about 13% w/w Dimethyl isosorbide, about 20% w/w Transcutol, about 10% w/w Hexylene glycol, about 4% w/w Propylene glycol, about 0.015% w/w Methylparaben, about 0.05% w/w Propylparaben, about 0.01% w/w EDTA, about 0.3% w/w Carbopol Ultrez 10, about 0.2% w/w Penmulen TR-1, about 2% w/w Cetyl alcohol, about 5.5% w/w Light mineral oil, about 5% w/w Oleic acid, and about 0.2% w/w Butylated hydroxytoluene.

49. The topical formulation of claim 1, wherein the topical formulation comprises at least of: a skin penetration agent, a gelling agent, an emollient, an anti-oxidant, a skin protecting agent, a dry-feel modifier, a surfactant, a preservative, a chelating agent, a lubricant, a thickening agent, at least one additional therapeutic agent.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The topical formulation of claim 49, wherein the additional therapeutic agent is an antioxidant, anti-inflammatory agent, antiangiogenic agent, anti-apoptotic agent, vascular endothelial growth factor inhibitor, antimicrobial or antiviral agent.

64-194. (canceled)
